(19) United States
(12) Patent Application Publication
(10) Pub. No.: US 2011/0274651 A1
(43) Pub. Date: Nov. 10, 2011

(54) Noscapine and Noscapine Analogs and Their Use in treating Infectious Diseases by Tubulin Binding Inhibition

(75) Inventors: Cory Acuff, Lawrenceville, GA (US); Daniel Kalman, Atlanta, GA (US)

(73) Assignee: Emory University, Atlanta, GA (US)

(21) Appl. No.: 13/061,447

(22) PCT Filed: Sep. 4, 2009
(86) PCT No.: PCT/US09/56075

§ 371 Date: Jul. 29, 2011

Related U.S. Application Data
(60) Provisional application No. 61/096,245, filed on Sep. 11, 2008.

(51) Int. Cl. A61K 31/4355; A61K 31/5377; A61K 31/4725; A61K 31/506; A61K 39/395; A61K 31/7088; A61K 31/7105; A61K 38/21; A61K 31/7028; A61K 31/4353; A61K 31/407; A61K 31/545; A61K 38/02; A61K 31/397; A61K 31/43; A61K 31/65; A61K 31/7048; A61K 31/513; A61P 31/00; A61P 31/14; A61P 31/12; A61P 31/20; A61P 31/22; A61P 31/16; A61P 31/18; A61P 31/04; A61P 31/10; A61K 31/496

(52) U.S. Cl. 424/85.4; 514/291; 514/253.03; 514/232.8; 514/256; 424/131.1; 514/44 A; 514/44 R; 514/25; 514/210.04; 514/210.09; 514/200; 514/3.1; 514/210.02; 514/192; 514/154; 514/2.4; 514/31; 514/254.07; 514/274

(57) ABSTRACT

Compositions and methods for treating or preventing infectious diseases, and inhibiting the ability of microbes to travel within mammalian cells, and inhibiting microbial replication, are disclosed. The compositions include various noscapine analogs, which are capable of blocking the movement of viruses and other microbes within mammalian and other cells by inhibiting the cytoplasmic transport mechanisms within the cells. The compositions described herein include an effective amount of the noscapine analogues described herein, along with a pharmaceutically acceptable carrier or excipient. The compositions can also include one or more additional antimicrobial compounds.

The government has certain rights to this invention by virtue of NIH Grant No. R56A 1058961-01A2.

### FIELD OF THE INVENTION

The present invention relates to noscapine and noscapine analogs, pharmaceutical compositions incorporating the noscapine and noscapine analogs, and methods of using the compounds and compositions to treat infectious diseases. This application provides methods for treating infectious disease organisms using noscapine and noscapine analogs as tubulin binding inhibitors, alone or in combination with other antimicrobial agents.

### BACKGROUND OF THE INVENTION

Microtubule-mediated transport of macromolecules and organelles is essential for cells to function. Deficiencies in cytoplasmic transport are frequently associated with severe diseases and syndromes. Cytoplasmic transport also provides viruses with the means to reach their site of replication and is the route for newly assembled progeny to leave the infected cell. (Greber, Urs F. and Way, Michael (Feb. 24, 2006) A Superhighway to Virus Infection. Cell 124, 741-754) During their life cycle, viruses spread from cell to cell, and must get from the plasma membrane to their site of replication and back again after replication. This can be a problem, since the size of viruses and the high density of the cytoplasm precludes efficient directional movements by free diffusion (Greber, Urs F. and Way, Michael (Feb. 24, 2006) A Superhighway to Virus Infection. Cell 124, 741-754).

The transport of viruses through the cell by diffusion is believed to be relatively slow (Sodeik, B. (2000). Mechanisms of viral transport in the cytoplasm. Trends Microbiol. 8, 465-472). Furthermore, random diffusional movements are unlikely to drive virus particles to their desired destinations, thus reducing the speed of infection and overall viral fitness. Therefore, viruses have evolved efficient mechanisms to hijack the cellular transport systems of their unwilling hosts. (Greber, Urs F. and Way, Michael (Feb. 24, 2006) A Superhighway to Virus Infection. Cell 124, 741-754)

It is believed that all viruses use cytoskeletal and motor functions in their life cycles. Viruses use the intracellular machinery of the cell for transport, including the microtubules within the cell, to aid their transportation and replication (Radtke, Kerstin, Dohner, Katinka, and Sodeik, Beate (2006) Viral interactions with the cytoskeleton: a hitchhiker's guide to the cell. Cellular Microbiology (3), 387-400). Certain bacteria and fungi are also known to use microtubules to infect cells. The transportation mechanism is also described, for example, in Yoshida et al. Exploiting host microtubule dynamics: a new aspect of bacterial invasion. Trends Microbiol. (2003) vol. 11 (3) pp. 139-43; Guignot et al. Microtubule motors control membrane dynamics of Salmonella-containing vacuoles. J Cell Sci (2004) vol. 117 (Pt 7) pp. 1033-45; Jouvenet et al. Transport of African swine fever virus from assembly sites to the plasma membrane is dependent on microtubules and conventional kinesin. Journal of Virology (2004) vol. 78 (15) pp.

7990-8001; Ruthel et al. Association of ebola virus matrix protein VP40 with microtubules. Journal of Virology (2005) vol. 79 (8) pp. 4709-19; and Eash et al. Involvement of cytoskeletal components in BK virus infectious entry. J. Virol. (2005) vol. 79 (18) pp. 11734-41.

Viral and bacterial infections are typically treated using conventional antimicrobial compounds, such as antiviral and antibacterial compounds, which kill the viruses or bacteria. However, while these agents are seeking to kill existing viruses and bacteria, it would be useful to find ways of preventing or inhibiting microbial replication, growth and/or proliferation within the cell.

It would therefore be advantageous to develop compositions and methods for using compounds that inhibit the ability of microbes to attach to tubulin, to treat, prevent, or otherwise inhibit microbial replication, growth, and/or proliferation. The present invention provides such compositions and methods.

### SUMMARY OF THE INVENTION

Compositions and methods for treating or preventing infectious diseases, and inhibiting the ability of microbes to travel within mammalian cells, are disclosed. The compositions include noscapine and various noscapine analogs, which are capable of blocking the movement of viruses and other microbes within mammalian and other cells by inhibiting the cytoplasmic transport mechanisms within the cells.

Noscapine ((S)-6,7-dimethoxy-3-((R)-4-methoxy-6-methyl-5,6,7,8-tetrahydro[1,3]-dioxolo-[4,5-g]isoquinolin-5-yl)isobenzo-furan-1(3H)-one), a safe antitussive agent used for over 40 years, is known to bind tubulin. Tubilin binding can inhibit the ability of microbes, such as viruses and bacteria, to travel within the cell. Unlike other microtubule-targeting drugs, noscapine does not significantly change the microtubule polymer mass even at high concentrations. Instead, it suppresses microtubule dynamics by increasing the time that microtubules spend in an attenuated (pause) state when neither microtubule growth nor shortening is detectable (Landen J W, Hau V, Wang M S, Davis T, Ciliax B, Wainer B H, Van Meir E G, Glass J D, Joshi H C, Archer D R. Noscapine Crosses the Blood-brain Barrier and Inhibits Glioblastoma Growth. Clin Cancer Res 2004; 10:5187-5201).

Noscapine, and the noscapine analogues described in this application, are also capable of blocking the movement of viruses and other microbes within the cells, by inhibiting the cytoplasmic transport mechanisms within the cells. Noscapine and these noscapine analogues, and pharmaceutical compositions including these compounds, inhibit the movement of the disease-causing organisms, and, accordingly, slow their replication. Because the noscapine analogs inhibit tubulin binding by the virus or other microbe, and therefore prevent the virus or other microbe from hijacking the cytoskeletal machinery of the cell, one can slow the growth and proliferation of the virus or other microbe, and allow for antimicrobial agents and/or the body's own immune responses, such as antibodies, phagocytosis, and the like, to treat the infection.

The compositions described herein include an effective amount of noscapine and/or the noscapine analogues described herein, along with a pharmaceutically acceptable carrier or excipient. When employed in effective amounts, the compounds can act as a therapeutic or prophylactic agent to inhibit the replication of a variety of microbes, including viruses, bacteria, fungi, and the like. This inhibition can help treat or prevent a wide variety of infectious diseases, including retroviral infections (HIV and the like), hepatitis B, hepatitis C, herpes, and the like.

The compositions can also include one or more antimicrobial compounds, which treat microbial infections by another method, such as inhibiting enzymes or receptors within the bacteria, penetrating bacterial cell walls, inhibiting viral replication by incorporating unnatural nucleosides into the growing DNA strands during replication, and the like.

The foregoing and other aspects of the present invention are explained in detail in the detailed description and examples set forth below.

### BRIEF DESCRIPTION OF THE FIGURES

FIG. 1 is a graph showing photographs of BSC-40 cells subjected to vaccinia virus and left untreated (control) or treated with DMSO (0.1% carrier) or 25 μM Br-Noscapine in 0.1% DMSO. Clear areas in control and DMSO treated monolayers represent areas where infected cells have lysed.

FIG. 2 is a photograph of a single 120 nm optical section from a confocal laser scanning microscope showing the microtubule cytoskeleton (green) of a HeLa cell infected with Texas red-labeled Ad2 particles (red) for 30 min. Enlarged insets highlight the colocalization of Ad2 particles (arrowheads) with microtubules in the periphery of the cell. Bars, 10 mm and 2 mm (inset).

FIGS. 3A and 3B are photographs showing adenoviruses tagged with a few fluorophores on each of the 252 copies of the capsid hexon trimer associated with microtubules inside a cell, showing that membrane-associated cytoplasmic HSV capsids bind to microtubules in vitro.

FIG. 3A is a photograph showing bouyant organelles isolated from the cytoplasm of HSV K26GFP-infected cells, and flowed into an imaging chamber with pre-bound rhodamine-labeled microtubules. After an incubation of 5 to 10 min, unbound material was washed away, and the chamber was imaged using fluorescence microscopy. The upper panel shows microtubules in red and bound HSV-containing organelles in green. The lower panel is another representative field shown in black and white. Scale bar, 10.

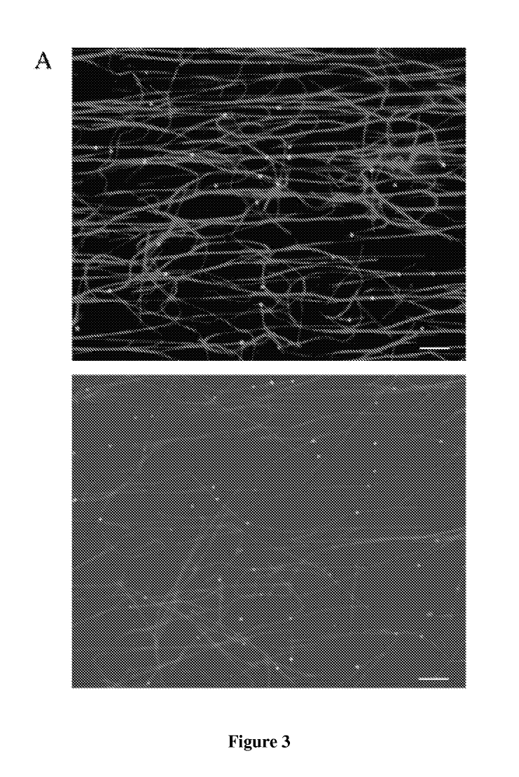

In FIG. 3B, HSV was bound to microtubules as in FIG. 3A, and the chamber was then fixed in glutaraldehyde and prepared for transmission electron microscopy. This representative image appears to show HSV capsids partially or completely enclosed by an organelle (arrowhead) or adjacent to an organelle (black arrow) and in both cases attached to a microtubule (white arrow). Scale bar, 100 nM.

### DETAILED DESCRIPTION

Compositions and methods for inhibiting viral and other microbial replication, and for treating and/or preventing viral and other microbial infection, are disclosed.

Viruses, which range from about 20 to several hundred nanometers, are obligate parasites, as their genomes do not encode all the proteins required for replication. Viruses can manipulate cellular functions of their host (such as a human) to achieve replication. Certain of these functions include the ability to inhibit cellular apoptosis during replication, while at the same time minimizing detection by host immune surveillance systems. Viral transport is also essential, and viruses must get from the plasma membrane to their site of replication and back again after replication. Viruses use the microtubule cytoskeleton to effectively transport themselves within the cells. The compounds described herein inhibit the ability of viruses and other microbes from using the microtubule cytoskeleton to transport themselves within the cells.

### DEFINITIONS

The present invention will be better understood with reference to the following definitions:

As used herein, alkyl refers to C₁₋₈ straight, branched, or cyclic alkyl groups, and alkenyl and alkynyl refers to C₂₋₈ straight, branched or cyclic moieties that include a double or triple bond, respectively. Aryl groups include C₆₋₁₀ aryl moieties, specifically including benzene. Heterocyclic groups include C₃₋₁₀ rings which include one or more O, N, or S atoms. Alkylaryl groups are alkyl groups with an aryl moiety, and the linkage to the nitrogen at the 9-position on the noscapine framework is through a position on the alkyl group. Arylalkyl groups are aryl groups with an alkyl moiety, and the linkage to the nitrogen at the 9-position on the noscapine framework is through a position on the aryl group. Aralkenyl and aralkynyl groups are similar to aralkyl groups, except that instead of an alkyl moiety, these include an alkenyl or alkynyl moiety. Substituents for each of these moieties include halo, nitro, amine, thio, hydroxy, ester, thioester, ether, aryl, alkyl, carboxy, amide, azo, and sulfonyl.

### I. Compounds

The compounds are noscapine and noscapine analogs, prodrugs or metabolites of these compounds, and pharmaceutically acceptable salts thereof. The compounds generally fall within one of the two formulas provided below:

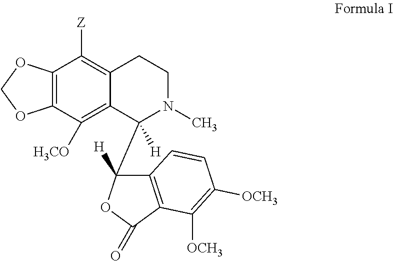

wherein Z is, individually, selected from the group consisting of H, alkyl, substituted alkyl, alkenyl, substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, —OR′, —NR′R″, —CF₃, —CN, —C₂R′, —SW, —N₃, —C(═O)NR′R″, —NR′C(═O)R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)OR″, —SO₂R′, —SO₂NR′R″, and —NR′SO₂R″,

where R′ and R″ are individually hydrogen, C₁-C₈ alkyl, cycloalkyl, heterocyclyl, aryl, or arylalkyl, and r is an integer from 1 to 6,

wherein the term “substituted” as applied to alkyl, aryl, cycloalkyl and the like refers to the substituents described above, starting with alkyl and ending with —NR′SO₂R″; and

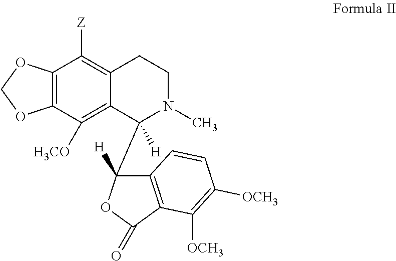

wherein Z is nitro, amino, bromo, chloro, iodo, or fluoro.

The compounds of both formulas can occur in varying degrees of enantiomeric excess.

The compounds can be in a free base form or in a salt form (e.g., as pharmaceutically acceptable salts). Examples of suitable pharmaceutically acceptable salts include inorganic acid addition salts such as sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with an acidic amino acid such as aspartate and glutamate; alkali metal salts such as sodium and potassium; alkaline earth metal salts such as magnesium and calcium; ammonium salt; organic basic salts such as trimethylamine, triethylamine, pyridine, picoline, dicyclohexylamine, and N,N′-dibenzylethylenediamine; and salts with a basic amino acid such as lysine and arginine. The salts can be in some cases hydrates or ethanol solvates. The stoichiometry of the salt will vary with the nature of the components.

Pharmaceutically acceptable salts may be obtained using standard procedures well known in the art, for example by reacting an amine group with a suitable acid affording a physiologically acceptable anion. In one embodiment, the salt is a hydrochloride salt of the compound.

Representative compounds include the following:
Noscapine-((S)-6,7-dimethoxy-3-((R)-4-methoxy-6-methyl-5,6,7,8-tetrahydro-[1,3]dioxolo[4,5-g]isoquinolin-5-yl)isobenzofuran-1(3H)-one)
9-Nitro-Nos((S)-6,7-dimethoxy-3-((R)-4-methoxy-6-methyl-9-nitro-5,6,7,8-tetrahydro-[1,3]dioxolo[4,5-g]isoquinolin-5-yl)isobenzofuran-1(3H)-one)
9-Amino-Nos((S)-6,7-dimethoxy-3-((R)-4-methoxy-6-methyl-9-nitro-5,6,7,8-tetrahydro-[1,3]dioxolo[4,5-g]isoquinolin-5-yl)isobenzofuran-1(3H)-one)
9-Chloro-Nos((S)-6,7-dimethoxy-3-((R)-4-methoxy-6-methyl-9-nitro-5,6,7,8-tetrahydro-[1,3]dioxolo[4,5-g]isoquinolin-5-yl)isobenzofuran-1(3H)-one)
9-Iodo-Nos((S)-6,7-dimethoxy-3-((R)-4-methoxy-6-methyl-9-iodo-5,6,7,8-tetrahydro-[1,3]dioxolo[4,5-g]isoquinolin-5-yl)isobenzofuran-1(3H)-one)
9-Bromo-Nos((S)-6,7-dimethoxy-3-((R)-4-methoxy-6-methyl-9-bromo-5,6,7,8-tetrahydro-[1,3]dioxolo[4,5-g]isoquinolin-5-yl)isobenzofuran-1(3H)-one) and
9-Fluoro-Nos((S)-6,7-dimethoxy-3-((R)-4-methoxy-6-methyl-9-fluoro-5,6,7,8-tetrahydro-[1,3]dioxolo[4,5-g]isoquinolin-5-yl)isobenzofuran-1(3H)-one),
   prodrugs or metabolites of these compounds, and pharmaceutically acceptable salts thereof.

9-Chloro-noscapine has the structure shown below.

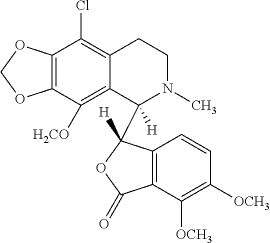

9-amino-noscapine has the structure shown below.

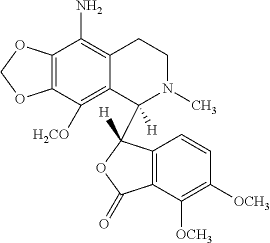

Also within the scope of the invention are compounds of the following Formula V, as described in PCT WO 2007/133112 A1, the contents of which are hereby incorporated by reference.

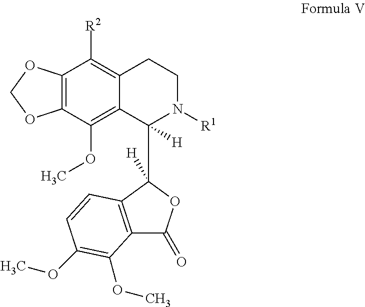

Wherein R¹ is an amino group, and R² is a cyclic system substituent selected from possibly substituted alkyl, wherein the substituents are selected from a optionally substituted amino group, or azaheterocycle, which optionally contains O, S, or N in the form of an additional heteroatom and linked to an alkyl group by a nitrogen atom, from optionally substituted aryl, optionally substituted and optionally contensed heteroaryl containing at least one heteroatom selected from nitrogen, sulfur and oxygen, and optionally substituted sulfamoyl.

Amino groups can include one or more substituents such as hydrogen, alkyl, aryl, aralkyl, heteroaralkyl, heterocyclyl either heteroaryl or Rka and Rk+ia together with the atom N, with which they are connected, form through Rka and Rk+i4 a 4-7 member heterocyclyl or heterocyclenyl ring. Preferred alkyl groups are methyl, trifluoromethyl, cyclopropylmethyl, cyclopentylmethyl, ethyl, n-propyl, isopropyl, n-butyl, tert-butyl, pentyl, 3-pentyl, methoxyethyl, carboxymethyl, methoxycarbonylmethyl, ethoxycarbonylmethyl, benzylhydroxycarbonylmethyl methoxycarbonylmethyl and pyridilmethyloxycarbonylmethyl.

Preferred cyclic system substituent also include cycloalkyl, aryl, heteroaryl, heterocyclyl, hydroxy, alkoxy, alkoxycarbonyl, aryloxy, arylhydroxy, alkylthio, heteroarylthio, aralkylthio, alkylsulfonyl, arylsulfonyl, alkoxycarbonyl, aryloxycarbonyl, heteroaralkylhydroxycarbonyl or RkaRk+iaN—, RkaRk+ïaNC(═O)—, annelated arylheterocyclenyl, and annelated arylheterocyclyl.

“Alkyloxyalkyl” indicates alkyl-O— Alkyl the group, in which alkyl groups are independent from each other and are determined in this application. Preferred alkylhydroxyalkyl groups are methoxyethyl, ethoxymethyl, butoxymethyl, methoxypropyl, also, from -propyloxyethyl. “alkyloxyalkonyl,” indicates alkyl-O—C (═O) the group, in which alkyl groups are determined in this application.

Preferred alkyl hydroxycarbonyl groups are methoxycarbonyl, ethoxycarbonyl, butoxycarbonyl tert-butylhydroxycarbonyl, isopropylhydroxycarbonyl, benzylcarbonyl and phenethylcarbonyl. “Alklthio” indicates alkyl-S the group, in which alkyl the group is determined in this application. “Alkyloxy” indicates alkyl-0 the group, in which alkyl is determined in this application. Preferred alkylhydroxy by groups are methoxy, ethoxy, n-propoxy, iso-propoxy and butoxy. “

Alkoxycarbonylalkyl” indicates alkyl-O—C(═O)-alkyl- the group, in which alkyl is determined in this application. Preferred alkoxycarbonylalkylnymi groups are methoxycarbonylmethyl and ethoxycarbonylmethyl and methoxy-carbonylethyl and ethoxycarbonylethyl.

“Amino group”, indicates a substituted or un-substituted N(Rka)(Rk+1)- group.

Examples of amino groups, Rka and Rk+1 value of which is determined in this application, for example, of amino (H₂N—), methylamino, diethylamine, pyrrolidine, morpholine, benzylamine or phenethyl.

“Amino acids” indicates natural amino acid or unnatural amino acid, the value of the latter is determined in this application. Preferred amino acids are the amino acids, which contain α- or β-amino group. α-amino acids are an example of natural amino acids, as them can serve alanine, valine, leucine, isoleucine, proline, phenylalanine, tryptophan, metionine, glycine, series, threonine and cysteine.

“Annelated cycle” (condensed cycle) indicates the bi- or multicycle system, in which the annelated cycle and cycle or the poly-cycle, with which it “annelates”, have as the minimum two general atoms.

“Annelated arylheterocycloalkenyl” indicates annelated aryl and heterocycloalkenyl, whose value is determined in this application.

Annelated arylheterocyclylalkenyl can be connected through any possible atom of cyclic system. The prefix “of aza”, “oxa” or “thia” before “heterocyclylalkenyl” indicates the presence in the cyclic system of the atom of nitrogen, atom of oxygen or atom of sulfur, respectively. Annelated arylheterocyclylalkenyl can have one or several of “types of cyclic system)), which can be identical or different. Atoms of nitrogen and sulfur, which are found in the heterocyclenyloyl part can be oxidized to the N-oxide, the S-oxide or the S-dioxide. The representatives of annelated arylheterocyclylalkenoyl are indolineyl, SH-2-alkoxyinolinyl, 2H-1 oxoisoquinolinyl, 1,2-dihydroxinolinyl and the like.

“Annelated arylheterocycloalkyl” indicates annelated aryl and heterocyclylalkyl, whose value is determined in this application. Annelated arylheterocycloalkyl can be connected through any possible atom of cyclic system. The prefix “of aza”, “oxa” or “thia” before “heterocycloalkyl” indicates the presence in the cyclic system of the atom of nitrogen, atom of oxygen or atom of sulfur, respectively.

Annelated arylheterocycloalkyl can have one or several of “types of cyclic system)), which can be identical or different. Atoms of nitrogen and sulfur, which are found in the heterocyclyll part can be oxidized to N-oxide, S of oxide or S-dioxide. The representatives of annelated arylheterocycloalkyl are indolyl, 1,2,3,4-tetrahydroisoxinolyn, 1,3-benzodiokol and the like.

“Annelated aryl cycloalkenyl” indicates annelated aryl and cycloalkenyl, whose value is determined in this application. Annelated arylcycloalkenyl can be connected through any possible atom of the cyclic system. Annelated arylcycloalkenyl can have one or several “types of cyclic systems”, which can be identical or different.

Representatives annelated arylcycloalkenyls include 1,2-dihydronaphthalene, indene and the like “Annelated arylcycloalkyl” indicates annelated aryl and cycloalkyl, whose value is determined in this application. Annelated arylcycloalkyl can be connected through any possible atom of cyclic system. Annelated arylcycloalkyl can have one or several of “types of cyclic systems”, which can be identical or different. The representatives of annelated arylcycloalkylov are indane, 1,2,3,4 tetrahydronaphthalene, 5,6,7,8-tetrahydronaphth-1-il. and the like.

“Annelated heteroarylcycloalkenyl heteroarylcycloalkenyl” indicates annelated heteroaryl and cycloalkenyl, whose values are determined in this application. Annelated heteroarylcycloalkenyl can be connected through any possible atom of cyclic system. The prefix “of aza”, “oxa” or “thia” before “heteroaryl” indicates the presence in the cyclic system of the atom of nitrogen, atom of oxygen or atom of sulfur, respectively. Annelated heteroarylcycloalkenyl can have one or several types of cyclic systems, which can be identical or different. The nitrogen atom, located in the heteroaryl part, can be oxidized to the N-oxide. Representative annelated heteroarylcycloalkenyls are 5,6-dihydroquinolinyl, 5,6-dihydroisoquinolinyl, 4,5-dihydro-1H-benimidazolyl and the like.

“Annelated heteroarylcycloalkyl” indicates annelated heteroaryl and cycloalkyl, whose values are determined in this application. Annelated heteroarylcycloalkyl can be connected through any possible atom of cyclic system. The prefix “of aza”, “oxa” or “thia” before “heteroaryl” indicates the presence in the cyclic system of the atom of nitrogen, atom of oxygen or atom of sulfur, respectively. Annelated heteroarylcycloalkyl can have one or several types of cyclic systems, which can be identical or different. The nitrogen atom located in the heteroaryl part can be oxidized to the N-oxide.

Representatives annelated heteroarylcycloalkyls include 5,6,7,8-tetrahydroquinolineyl, 5,6,7,8-tetrahydroisoxinolynyl, 4,5,6,7-tetrahydro-IH-benzimidazolyl and the like.

“Annelated heteroarylheterocyclenyl” indicates annelated heteroaryl and heterocyclenyl, whose values are determined in this application. Annelated heteroarylheterocyclenyl can be connected through any possible atom of cyclic system. The prefix “of aza”, “oxa” or “thia” before “heteroaryl” indicates the presence in the cyclic system of the atom of nitrogen, atom of oxygen or atom of sulfur, respectively.

Annelated heteroarylheterocyclenyl can have one or several of types of cyclic systems, which can be identical or different. The nitrogen atom located in the heteroaryl part can be oxidized to the N-oxide. Atoms of nitrogen and sulfur, which are found in the heterocyclenyl part can be oxidized to the N-oxide, the S-oxide or the S-dioxide. Representative annelated heteroarylheterocyclenyl include 1,2-dihydro 2,7 naphthyridinyl, 7,8-dihydro 1, 7 naphthyridinyl, 6,7-dihydro-3H-imidazo 4,5-c of pyridyl and the like “annelated heteroarylheterocyclyl” indicates annelated heteroaryl and heterocyclyl, whose values are determined in this application.

Annelated heteroarylheterocyclyl can be connected through any possible atom of cyclic system. The prefix “of aza”, “oxa” or “thia” before “heteroaryl” indicates the presence in the cyclic system of the atom of nitrogen, atom of oxygen or atom of sulfur, respectively. Annelated heteroarylheterocyclyl can have one or several of “types of cyclic systems”, which can be identical or different. The nitrogen atom located in the heteroaryl part can be oxidized to the N-oxide. Atoms of nitrogen and sulfur, which are found in the heterocyclyl part can be oxidized to the N-oxide, the S-oxide or the S-dioxide. The representatives of annelated heteroarylheterocyclylov are 2,3-dihydro-Sh-pyrrolo 3,4-b xinolin-2-yl, 2,3-dihydro-Sh-pyrrolo 3,4-b indol-2-yl, 1,2,3,4-tetrahydro 1,5 naphthyridinyl and the like “aralkenyl” indicates aryl-alkenyl the group, in which the values aryl and alkenyl are determined in this application. For example, 2-fenetenyl is aralkenyl group.

“Aralkyl” indicates the alkyl group, substituted by one or several aryl groups, in which the values aryl and alkyl are determined in this application. Examples of aralkyl groups are benzyl, 2,2-diphenylethyl or phenethyl.

“Aralkylamino” indicates aryl-alkyl —NN the group, in which the values aryl and alkyl are determined in this application.

“Aralkylsulfonyl” indicates aralkyl —SO the group, in which the value aralkyl is determined in this application.

“Aralkylsulfonyl” indicates aralkyl-SO₂— the group, in which the value aralkyl is determined in this application.

“Aralkylthio” indicates aralkyl-S the group, in which the value aralkyl is determined in this application.

“Aralkyloxy” indicates aralkyl-0 the group, in which the value aralkyl is determined in this application. For example, benzylhydroxy or 1 or 2-naphthylenmethoxy are aralkyl groups.

“Aralkyloxyalkyl” indicates aralkyl-O— Alkyl the group, in which the values aralkyl and alkyl are determined in this application. An example of aralkyl-O-alkyl group is benziloxyethyl.

“Aralkoxycarbonyl” indicates aralkyl-O—C(═O)— the group, in which the value aralkyl is determined in this application. An example of aryloxycarbonylnoy group is benzylhydroxycarbonyl.

“Aralkoxycarbonylalkyl” indicates aralkyl-O—C(═O)-alkyl- the group, in which the values aralkyl and alkyl are determined in this application. An example of aryloxycarbonylalkylnoy group is benzylhydroxycarbonylmethyl or benzylhydroxycarbonylethyl.

“Aryl” indicates the aromatic monocyclic or multicycle system, which includes from 6 to 14 carbon atoms, preferably from 6 to 10 carbon atoms. Aryl can contain one or more “types of cyclic system)), which can be identical or different. The representatives of aryl groups are phenyl or naphthyl, substituted phenyl or substituted naphthyl. Aryl can be annelated with the nonaromatic cyclic system or the heterocycle.

“Arylcarbamoyl” indicates aryl-NHC(═O)— the group, in which the value aryl is determined in this application.

“Aryloxy” indicates aryl-0 the group, in which the value aryl is determined in this application. By the representatives arylhydroxy groups are phenoxy 2-naphthyloxy. “Aryloxycarbonyl” indicates aryl-O—C(═O)— the group, in which the value aryl is determined in this application. Representatives aryloxycarbonyl groups are phenoxycarbonyl and 2-naphthoxycarbonyl.

“Arylsulfonyl” indicates aryl —SO the group, in which the value aryl is determined in this application.

“Arylsulfonyl” indicates aryl-SO₂— the group, in which the value aryl is determined in this application. “Arylthio” indicates aryl-S the group, in which the value aryl is determined in this application. Representative arylthio groups are phenylthio and 2-naphthylthio.

“Aroylamino” indicates aroyl —N the group, in which the value aroyl is determined in this application. “Aroyl” indicates aryl-C(═O)— the group, in which the value aralkyl is determined in this application. Examples of aroyl groups are benzoyl, 1 y of 2-maphthoyl.

“Aromatic” radical indicates the radical, obtained by the removal of hydrogen atom from the aromatic C—H of the compound.

“Aromatic” radical includes the aryl and heteroaryl cycles, determined in this application. Aryl and heteroaryl cycles can additionally contain groups—aliphatic or aromatic radicals, determined in this application. Representative aromatic radicals include aryl, annelated cycloalkenylaryl, annelated cycloalkaryl, annelated heterocyclylaryl, annelated heterocyclenylaryl, heteroaryl, annelated cycloalkylheteroaryl, annelated cycloalkenylheteroaryl, annelated heterocyclenylheteroaryl and annelated heterocyclylheteroaryl.

“Aromatic cycle” indicates the planar cyclic system, in which all atoms of cycle participate in the formation of the united conjugated system, which includes, according to Hueckel's rule, (4n+2) π-electrons (p the entire non-negative number). Examples of aromatic cycles are benzene, naphthalene, anthracene and the like.

In the case of heteroaromatic cycles in the conjugated system participate π-electrons and r the electrons of heteroatoms, their total number also is equal to (4n+2). Examples of such cycles are pyridine, thiophene, pyrrole, furan, thiazole and the like aromatic cycle can have one or more “types of cyclic)) system it can be annelated with the nonaromatic cycle, the heteroaromatic or heterocyclic system. “Oxo” indicates H—C(═O)— either alkyl-C(═O)—, cycloalkyl-C(═O)—, heterocyclyl-C (═O)—, heterocyclylalkyl-C(═O)—, aryl-C(═O)—, arylalkyl-C(═O)— or heteroaryl-C(═O)—, heteroarylalkyl-C(═O)— group, in which alkyl-, cycloalkyl, heterocyclyl-, heterocyclylalkyl, aryl, arylalkyl, heteroaryl, heteroarylalkyl are determined in this application.

“Oxoamino” indicates acyl —NN the group, in which the value acyl is determined in this application.

“Bifunctional reagent” indicates the chemical compound, which has two reaction centers, that participate simultaneously or consecutively in the reactions. As an example of bifunctional reagents can serve the reagents, which contain carboxyl group and aldehyde or ketonic group is, for example, 2-formylbenzoic acid, is 2-(2-oxo-ethylcarbamoyl)-benzoic acid, is 2-(3-formyl-thiophene-2-yl)-benzoic acid or 2-(2-formylphenyl)-thiophene-3-carbonoxylic acid. “1,2-ethylenyl radical” indicates —CH═CH— the group, which contains one or several identical or different of the group “alkynyl”, whose value is determined in this application.

“Halogen” indicates fluorine, chlorine, bromine and iodine. Preferred are fluorine, chlorine and bromine.

“Heteroannelated cycle” means that the cycle, which is fastened (it annulates or it is condensed) to another cycle or poly-cycle, contains as the minimum one heteroatom.

“Heteroaralkenyl” indicates heteroarylalkenyl the group, in which heteroaryl and alkenyl are determined in this application. Preferably heteroarylalkenyl includes the lowest alkenyl group. Representative heteroarylalkenyls are pyridylvinyl, thienylethenyl, imidazolylethenyl, pyrazinylethenyl and the like.

“Heteroaralkyl” indicates heteroaryl-alkyl the group, in which heteroaryl and alkyl are determined in this application. The representatives heteroarylalkyl are pyridylmethyl, thienylmethyl, furylmethyl, imidazolylmethyl, pyrazineylmethyl and the like “heteroaralkyloxy” indicates heteroarylalkyl-0 the group, in which heteroarylalkyl is determined in this application. Preferred heteroarylalkylhydroxy groups are 4-pyridilmethyloxy, 2-thenylmethyloxy and the like

“Heteroaryoyl” indicates heteroaryl-C(═O)— the group, in which heteroaryl is determined in this application. The representatives heteroaroyls are nicotinyl, thienoyl, pyrazolyl and the like.

“Heteroaryl” indicates the aromatic monocyclic or multicycle system, which includes from 5 to 14 carbon atoms, preferably from 5 to 10, in which one or more than carbon atoms are substituted by heteroatom or heteroatoms, such as nitrogen, sulfur or oxygen.

The prefix “aza”, “oxa” or “thia” before “heteroaryl” indicates the presence in the cyclic system of the atom of nitrogen, atom of oxygen or atom of sulfur, respectively. Nitrogen atom, which is found in heteroaryl, can be oxidized to the N-oxide. Hetaryl can have one or several “types of cyclic systems”, which can be identical or different. Representative heteroaryls are pyrroleyl, furanyl, thienyl, pyridyl, pyrazinyl, pyrimidinyl, isooxazolyl, isothiazolyl, tetrazoleyl, oxazolyl, thiazolyl, pyrazolyl, furazanyl, triazolyl, 1,2,4-thiadiazolyl, pyridazinyl, quinoxalinyl, phthalazinyl, imidazo 1,2a pyrindyl, imidazo 2,1-b thiazolyl, benzofurazanyl, indolyl, azaindolyl, benzimidazolyl, benzothiazenyl, quinolineyl, imidazolyl, thienopyridil, quinazolinyl, thienopyrimidinyl, pyrrolepyridine, imidazopyridyl, isoquinolinyl, benzoazaindolyl, 1,2,4-triazinyl, thienopyrrolyl, furopyrrolyl, etc.

“Heteroarylsulfonylcarbamoyl” indicates heteroaryl-SO₂—NH—C(═O)— the group, in which heteroaryl is determined in this application.

“Heterocyclenyl” indicates the nonaromatic monocyclic or multicycle system, which includes from 3 to 13 carbon atoms, predominantly from 5 to 13 carbon atoms, in which one or several carbon atoms are substituted to the heteroatom such as nitrogen, oxygen, sulfur and which contains, at least, one carbon-carbon double bond or carbon-nitrogen double bond.

The prefix “aza”, “oxa” or “thia” before heterocyclenyl indicates the presence in the cyclic system of the atom of nitrogen, atom of oxygen or atom of sulfur, respectively. Heterocyclenyl can have one or several “types of cyclic systems”, which can be identical or different. Nitrogen and sulfur atoms, which are found in heterocyclenyl, can be oxidized to the N-oxide, the S-oxide or the S-dioxide. Representative heterocyclenyls are 1,2,3,4-tetrahydropyridine, 1,2-dihydropyridine, 1,4-dihydropyridine, 2-pippolinyl, 3-pippolinyl, 2-imidazolyl, 2-pipazolinyl, dihydrofuranyl, dihydrothiophenyl and the like.

“Heterocyclyl” indicates the nonaromatic saturated monocyclic or multicycle system, which includes from 3 to 10 carbon atoms, predominantly from 5 to 6 carbon atoms, in which one or several carbon atoms are substituted to the heteroatom, this as nitrogen, oxygen, sulfur.

The prefix “aza”, “oxa” or “thia” before heterocyclyl indicates the presence in the cyclic system of the atom of nitrogen, atom of oxygen or atom of sulfur, respectively. Heterocyclyl can have one or several types of cyclic systems, which can be identical or different. Atoms of nitrogen and sulfur, which are found in heterocyclyle, can be oxidized to N-oxide, S-oxide or S-dioxide. Representative heterocyclyls include piperidine, pyrrolidine, piperazine, morpholine, thiomorpholine, thiazolidine, 1,4-dioxan, tetrahydrofuran, tetrahydrothiophene and the like.

“Heterocyclyloxy” indicates the heterocyclyl-O— group, in which heterocyclyl is described in this application.

“Hydrate” indicates the solvate, in which the water is molecule or molecules of solvent.

“Hydroxyalkyl” indicates But-alkyl the group, in which alkyl is determined in this application.

“Radical” indicates the chemical radical, which is joined to scaffold (to fragment), for example, group is alkylnyl”, “radical amino group”, “radical is carbamoyl”, “radical cyclic systems”, whose values are determined in this application.

“Radical alkyl” indicates the group, connected to alkyl, to alkenyl, whose value is determined in this application. Substituent groups for alkyl include hydrogen, alkyl, halogen, alkenylhydroxy, cycloalkyl, aryl, heteroaryl, heterocyclyl, the aroyl, cyanogen, hydroxy, alkoxy, carboxy, alkyneylhydroxy, aryloxy, arylhydroxy, aryloxycarbonyl, alkylthio, heteroarylthio, aralkylthio, arylsulfonyl, alkylsulfonylheteroaralkyloxy, annelated heteroarylcycloalkenyl, annelated heteroarylcycloalkyl, annelated heteroarylheterocyclenyl, annelated heteroarylheterocyclyl, annelated arylcycloalkenyl, annelated arylcycloalkyl, annelated arylheterocyclenyl, annelated arylheterocyclyl, alkoxycarbonyl, aryloxycarbonyl, heteroaralkylhydroxycarbonyl or R/Rk+GN—, RkaR^{k+1}aNC(═O)—, RkaR^{k+1}NSO₂⁻, where R and R^{k+1}a independently of each other are “radical amino group”, whose value is determined in this application, for example, hydrogen atom, alkyl, aryl, aralkyl, heteroaralkyl, heterocyclyl either heteroaryl or Rka and Rk+ia together with the atom N, with which they are connected, form through Rka and Rk+ia 4-7 member heterocyclyl or heterocyclenyl.

Preferred alkyl groups are methyl, trifluoromethyl, cyclopropylmethyl, cyclopentylmethyl, ethyl, n-propyl, isopropyl, n-butyl, tert-butyl, pentyl, 3-pentil, methoxyethyl, carboxymethyl, methoxycarbonylmethyl, ethoxycarbonylmethyl, benzylhydroxycarbonylmethyl methoxycarbonylmethyl and pyridilmethyloxycarbonylmethyl.

Preferred “alkylinic groups” are cycloalkyl, aryl, heteroaryl, heterocyclyl, hydroxy, alkoxy, alkoxycarbonyl, aryloxy, arylhydroxy, alkylthio, heteroarylthio, aralkylthio, alkylsulfonyl, arylsulfonyl, alkoxycarbonyl, aryloxycarbonyl, heteroaralkylhydroxycarbonyl or R/Rk+̂N—, RkaR^{k+1}aNC(═O)—, annelated arylheterocyclenyl, annelated arylheterocyclyl. The value of the groups alkylnyx” is determined in this application.

The “amino group” can have various substituent groups connected to the nitrogen atom in the amino group. Examples include hydrogen, alkyl, cycloalkyl, aryl, heteroaryl, heterocyclyl, acyl, aroyl, alkylsulfonyl, arylsulfonyl, heteroarylsulfonyl, alkylaminecarbonyl, arylaminocarbonyl, heteroarylaminocarbonyl, heterocyclylaminocarbonyl, alkylaminethiocarbonyl, arylaminothiocarbonyl, heteroarylaminothiocarbonyl, heterocyclylaminothiocarbonyl, annelated heteroarylcycloalkenyl, annelated heteroarylcycloalkyl, annelated heteroarylheterocyclenyl, annelated heteroarylheterocyclyl, annelated arylcycloalkenyl, annelated arylcycloalkyl, annelated arylheterocyclenyl, annelated arylheterocyclyl, alkoxycarbonylalkyl, aryloxycarbonylalkyl, heteroaralkyloxycarbonylalkyl. The value “types of amino group” is determined in this application.

“Radical carbamoyl” indicates the group, connected to the carbamoyl group, whose value is determined in this application. Group carbamoyl is hydrogen, alkyl, cycloalkyl, aryl, heteroaryl, heterocyclyl, alkoxycarbonylalkyl, aryloxycarbonylalkyl, heteroaralkyloxycarbonylalkyl or R/Rk+̂N—, RkaRk+1aNC(═O)-alkyl annelated heteroarylcycloalkenyl, annelated heteroarylcycloalkyl, annelated heteroarylheterocyclenyl, annelated heteroarylheterocyclyl, annelated arylcycloalkenyl, annelated arylcycloalkyl, annelated arylheterocyclenyl, annelated arylheterocyclyl.

Preferred “radical carbamoyl groups” are alkyl, cycloalkyl, aryl, heteroaryl, heterocyclyl, alkoxycarbonylalkyl, aryloxycarbonylalkyl, heteroaralkyloxycarbonylalkyl or RkaRk+iaN—, RkaRk+iaNC(═O)-alkyl, annelated arylheterocyclenyl, annelated arylheterocyclyl. The value “types of carbamoyl” is determined in this application.

“Nucleophilic group” indicates the chemical radical, which is joined to scaffold as a result of reaction with the nucleophilic reagent by that, for example, selected from the group of primary or second amines, alcohols, phenols, mercaptans and thiophenols.

“Radical cyclic system” is the group, connected to the aromatic or nonaromatic cyclic system, examples of which include hydrogen, alkylalkenyl, alkyneyl, aryl, heteroaryl, aralkyl, heteroaralkyl, hydroxy, hydroxyalkyl, amino, aminoalkyl, alkoxy, arylhydroxy, acyl, aroyl, halogen, nitro, cyanogen, carboxy, alkoxycarbonyl, aryloxycarbonyl, aryloxycarbonyl, alkylhydroxyalkyl, arylhydroxyalkyl, heterocyclyloxyalkyl, arylalkyloxyalkyl, heterocyclylalkyloxyalkyl, alkylsulfonyl, arylsulfonyl, heterocyclylsulfonyl, alkylsulfinyl, arylsulfinyl, heterocyclylsulfinyl, alkylthio, arylthio, heterocyclylthio, alkylsulfonylalkyl, arylsulfonylalkyl, heterocyclylsulfonylalkyl, alkylsulfinylalkyl, arylsulfinylalkyl, heterocyclylsulfinylalkyl, alkylthioalkyl, arylthioalkyl, heterocyclylthioalkyl, arylalkylsulfonylalkyl, heterocyclylalkylsulfonylalkyl, arylalkylthioalkyl, heterocyclylalkylthioalkyl, cycloalkyl, cycloalkenyl, heterocyclyl, heterocyclenyl, amidine, RkaR^{k+1}aN—, RkaN═, RkaR^{k+1}aN-alkyl-, RkaR^{k+1}aNC(═O)— either RkaR^{k+1}aNSO₂⁻, where Rka and R^{k+1}a are, independently of each other, “radicals of amino groups”, whose value is determined in this application, for example, hydrogen, optionally substituted alkyl, optionally substituted aryl, optionally substituted aralkyl, or optionally substituted heteroaralkyl, or the group RkaR^{k+1}aN-, in which Rka can be acyl or aroyl, and the value of RSHA is determined above, or “radical cyclic systems” are RkaR^{k+1}aNC(═O)— or RkaR^{k+1}aNSO₂⁻, into which Rka and R^{k+1}a together with the atom, nitrogen with which they are connected, form through Rka and R^{k+1}a a 4-7 member heterocyclyl or heterocyclenyl.

“Radical electrophile” indicates the chemical radical, which is joined to scaffold as a result of reaction with the electrophilic reagent by that, for example, selected from the group of organic acids or of their derived (anhydrides, imidazolides, acid halides), ethers organic sulfo acids or organic sulfochlorides, organic haloformates, organic isoyanates and organic isothiocyanates. “zameshcheiiaya aminogroup” indicates RkaRk+1aN— the group, in which Rka and R^{k+1}a are the groups of the amino groups, whose value is determined in this application.

“Carboxyl group” indicates the C(O)OR— group. Group R has substituted carboxyl, including alkenyl, alkyl, aryl, heteroaryl, heterocyclyl, whose value is determined in this application.

“Mercapto group” indicates SR, S(O)R or S(O₂)R— group, in which the group R is alkenyl, alkyl, aryl, heteroaryl, heterocyclyl, whose value is determined in this application.

“Protecting group” (PG) indicates the chemical radical, which is joined to scaffold or half-finished product of synthesis for the temporary protection of amino group in the multifunctional compounds, including, but without limiting: amide group, this as formyl, not necessarily substituted acethyl (for example trichloroacethyl, trifluoroacetyl, 3-phenylpropionyl and other), not necessarily substituted benzoyl and other; carbamate group, this as not necessarily substituted by C₁₋₇ alkylhydroxycarbonyl, for example, methylhydroxycarbonyl, ethylhydroxycarbonyl, tert-butylhydroxycarbonyl, 9-fluorophenylmethyloxycarbonyl (Fmos) and other; the not necessarily substituted by C₁₋₇ alkyl group, for example, tert-butyl, benzyl, 2,4-dimethoxybenzyl, 9-phenylfluorophenyl and other; sulfonyl group, for example, benzenesulfonyl, p-toluolsulfonyl and other “protective groups” described in more detail in the book: Protective Groups in Organic Synthesis, Third Edition, Greene, T. W. and Wuts, P. G. M. 1999, r. 494-653. Publishing house John Wiley and Sons, New York, Chichester, Weipheim, Toropto, Singapore. Protected primary or second amine” indicates the group of the formula Of RkaR^{k+1}aN—, in which Rka is protecting group PG, and R^{k+1}a is hydrogen, “radical amino group”, whose value is determined in this application, for example, alkenyl, alkyl, aralkyl, aryl, annelated arylcycloalkenyl, annelated arylcycloalkyl, annelated arylheterocyclenyl, annelated arylheterocyclyl, cycloalkyl, cycloalkenyl, heteroaralkyl, heteroaryl, annelated heteroarylcycloalkenyl, annelated heteroarylcycloalkyl, annelated heteroarylheterocyclenyl, annelated heteroarylheterocyclyl, heterocyclenyl or heterocyclyl.

“Imino group”, indicates RkaN═ the group, substituted or unsubstituted “radical amino group” Rka, whose value is determined in this application, for example, of imine (HN═), methylimino (CH₃N═), ethylimino (C₂HN═), benzylimino (PhCH₂N═) or phenethylimino (PhCH₂CH₂N═). “Inactive group (or “Non-interfering substituent”) indicates low- or nonreactive radical, including, but without limiting C₁₋₇ alkyl, C₂₋₇ alkenyl, C₂₋₇ alkynyl, C₁₋₇ alkoxy, C₇₋₁₂ aralkyl, substituted by inert groups aralkyl, C₇₋₁₂ heterocyclylalkyl, substituted by the inert groups heterocyclylalkyl, C₇₋₁₂ alkaryl, C₃₋₁₀ cycloalkyl, C₃₋₁₀ cycloalkenyl, phenyl, substituted phenyl, toluoyl, xylenyl, biphenyl, C₂₋₁₂ alkoxyalkyl, C₂₋₁₀ alkylsulfinyl, C₂₋₁₀ alkylsulfonyl, (CH₂)mo(C₁₋₇ alkyl), (CH₂)Hi-N(C₁₋₇ alkyl)n, aryl, substituted by halogens, by inert groups aryl, substituted by the inert groups alkoxy, fluororalkyl, arylhydroxyalkyl, heterocyclyl, substituted by inert groups heterocyclyl and nitroalkyl; where t and p have a value from 1 to 7. Preferred “inactive groups are substituent groups such as C₁₋₇ alkyl, C₂₋₇ alkenyl, C₂₋₇ alkynyl, C₁₋₇ alkoxy, C₇₋₁₂ aralkyl, C₇₋₁₂ alkaryl, C₃₋₁₀ cycloalkyl, C₃₋₁₀ cycloalkenyl, substituted by inert groups C₁₋₇ alkyl, phenyl, substituted by inert groups phenyl, (CH₂)n, (C₁₋₇ alkyl), (CH₂)ᵣ—N (C₁₋₇ alkyl)n, aryl, substituted by inert groups aryl, heterocyclyl and substituted by inert groups heterocyclyl.

“Carbamoyl” indicates C(═O)nRkaR^{k+1}a- group. Carbamoyl can have one or some identical or different types of carbamoyl, Rka and Rk⁺¹a, including hydrogen, alkenyl, alkyl, aryl, heteroaryl, heterocyclyl, whose value is determined in this application.

“Carbamoylazaheterocycle” indicates azaheterocycle, which contains as “radicaly cyclic systems”, at least, one carbamoyl group.

The value “azaheterocycle”, “radical cyclic systems” and “carbamoyl group” are determined in this application. “Carboxyl” indicates HOC(═O)—(carboxyl) group.

“Carboxyalkyl” indicates HOC(═O)-alkyl- the group, in which the value alkyl is determined in this application.

“Carbocycle” indicates the mono- or multicycle system, which consists only of carbon atoms. Carbocycle can be both the aromatic and alicyclic.

Alicyclic polycycles can have one or more general common atoms. In the case of one general atom they are formed by spiro-carbocycle (for example, spiro 2.2 pentan), in the case of two—diverse to condensing system (for example, Decalin), in the case three—bridge systems (for example, bicyclo 3.3.1 nonane), in the case of the larger number—different polyhedral systems (for example, adamantane). Alicycles can be “saturated”, for example as cyclohexane, or “unsaturated)), for example as tetralin.

“Combinatorial library” indicates the collection of the connections, obtained by parallel synthesis, intended for lead generation or lead optimization, and also for the optimization of the physiological activity of Heath or leader, each connection of library corresponding to general scaffold, and library is the collection of the related homologues or analogs. “Methylenyl radical” indicates —CH2— the group, which contains one or two identical or different “radicalya alkylnyx”, whose value is determined in this application. “Heteroaromatic cycle” (saturated cycle or the partially saturated cycle) indicates the nonaromatic cyclic or multicycle system, formally formed as a result of complete or partial hydrogenation of unlimited C═C or C═N of connections.

Nonaromatic cycle can have one or more “types of cyclic)) system it can be annelated with the aromatic, heteroaromatic or heterocyclic systems. Cyclohexane or piperidine are examples of nonaromatic cycles, and cyclohexene is an example of a partially unsaturated cycle. “Unnatural aminocycle” indicates unnatural amino acids. By an example of unnatural amino acids can it serves the D-isomers of natural α-amino acids, amino-butyric acid, 2-aminomaclyanaya acid, γ-amino-butyric acid, the N-α-alkylated amino acids, 2,2-dialkyl-α-aminokicloty, 1-amino-cycloalkylcarboxylic acids, β-alanine, 2-alkyl-β-alaniny, 2-cycloalkyl-β-alaniny, 2-aryl-β-alaninyl, 2-heteroaryl-β-alanyl, 2-heterocyclyl-β-alaniny and (1-amino-cycloalkyl)-amino acids, in which the values alkyl, cycloalkyl, aryl, heteroaryl and heterocyclyl are determined in this application.

“Heterocycle aromatic cycle” indicates the cycle, which can be both the aromatic cycle and nonaromatic cycle, values of which are determined in this application.

“Hereocycle substituted radical” indicates radical without the groups or containing one or several groups.

“Annelated heterocycle (condensed) cycle” indicates the condensed, uncondensed cycle, whose value they are determined in this application. “Lower alkyl” indicates linear or branched alkyl with 1-4 carbon atoms.

“Parallel synthesis” indicates the method of conducting the chemical synthesis of the combinatory library of individual connections.

“1,3-Propylenyl radical” indicates —CH₂—CH₂—CH₂— the group, which contains one or several identical or different “types of alkylnyl”, whose value is determined in this application.

“Sulfamoyl group” indicates RkaR^{k+1}aNSO₂⁻ the group, substituted or unsubstituted “radical amino group” Rka and R^{k+1}a, whose values are determined in this application.

“Sulfonyl” indicates R—SO₂ the group, in which R is alkyl, cycloalkyl, aryl, heteroaryl, heterocyclyl, annelated heteroarylcycloalkenyl, annelated heteroarylcycloalkyl, annelated heteroarylheterocyclenyl, annelated heteroarylheterocyclyl, annelated arylcycloalkenyl, annelated arylcycloalkyl, annelated arylheterocyclenyl, annelated arylheterocyclyl, whose value is determined in this application.

“Template” indicates the general structural formula of the group of compounds or connections, entering in “to combinatorial library)).

“Thiocarbamoyl” indicates RkaR^{k+1}aNC(═S)— group. Thiocarbamoyl can have one or several identical or different “types of amino group” Rka and Rk⁻¹a, whose value specifically in this application, for example, including alkenyl, alkyl, aryl, heteroaryl, heterocyclyl, whose value is determined in this application.

“Cycloalkyl” indicates the nonaromatic mono- or multicycle system, which includes from 3 to 10 carbon atoms. Cycloalkyl can have one or several “types of cyclic system)), which can be identical or different. Representative cycloalkyl groups are cyclopropyl, cyclobutyl, cyclopentyl, cyclohexyl, decalin, norbornyl, adamant 1-yl and the like cycloalkyl can be annelated with the aromatic by cycle or heterocycle. By Preferred “cyclic system radicals include alkyl, aryloxy, hydroxy or RkaRk+iaN, whose value is determined in this application.

“Cycloalkylcarbonyl” indicates cycloalkyl-C(═O)— the group, in which the value cycloalkyl is determined in this application. The representative cycloalkylcarbonyl groups are cyclopropylcarbonyl or cyclohexylcarbonyl.

“Cycloalkyloxy” indicates cycloalkyl-0 the group, in which the value cycloalkyl is determined in this application.

The design of the focused libraries is, as a rule, connected with the directed search for the effectors (inhibitors, activators, agonists, antagonists the like) of those determined by bioactivity (ferments, receptors, ionic channels the like). “Fragment” (scaffold) indicates the structural formula of the part of the molecule, characteristic for the group of connections, or the molecular body, characteristic for the group of compounds or connections, entering in “to combinatorial library)). “1,2-Ethylenic radical” indicates—the group CH₂—CH₂—, which contains one or several identical or different “types of alkylnyl”, whose value is determined in this application.

The substituted Noscapine analogues of general Formula III, either by their racemates or their optical isomers, and their pharmaceutical acceptable salts and/or hydrates, are described in more detail below.

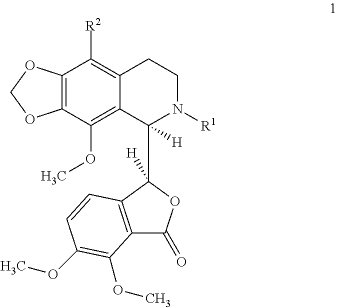

where: R¹ is an amino group, selected from alkyl; R² is a cyclic system, selected from optionally substituted alkyl, the optionally substituted aryl, optionally substituted and optionally condensed heteroaryl, which contains, at least, one heteroatom, selected from nitrogen, sulfur and oxygen, substituted possible sulfamoyl, excluding the compounds in which R═H, CH₃, 3-chlorphenylaminocarbonyl, R═Br; R═CH₃, R2=C₁, NO₂, CH₂OH, CH₃C(O), CO₂CH₃, CH₂NHC(O)CH₂Cl, 2-piperidin-1-yl-ethyl aminomethyl, 2-morpholin-4-yl-ethyl-aminomethyl, oxooxymethyl.

Individual compounds include compounds A1-20

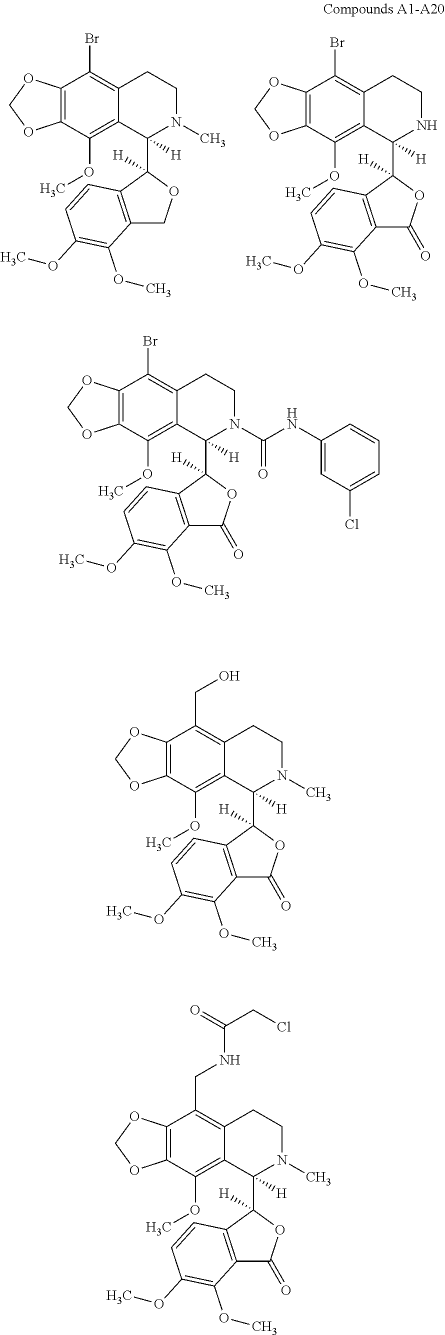

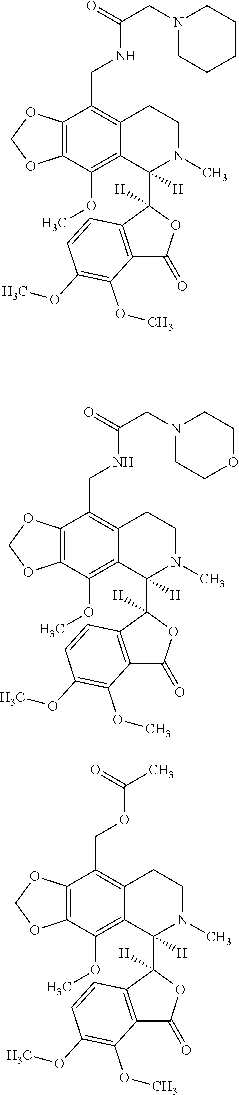

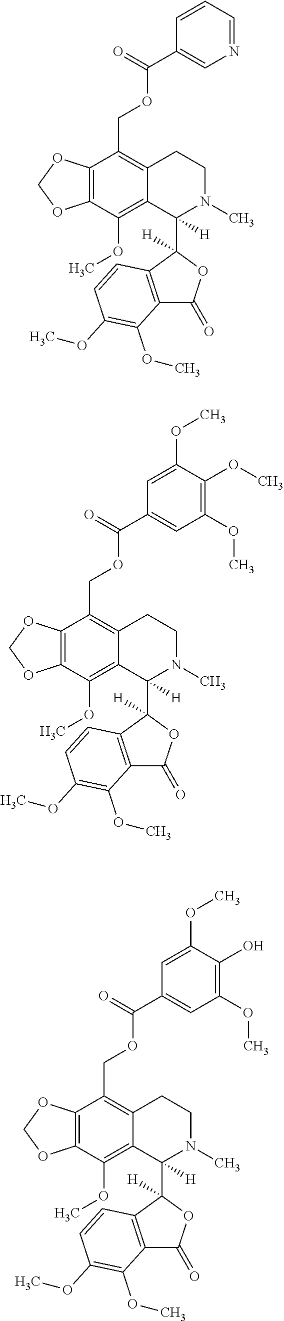

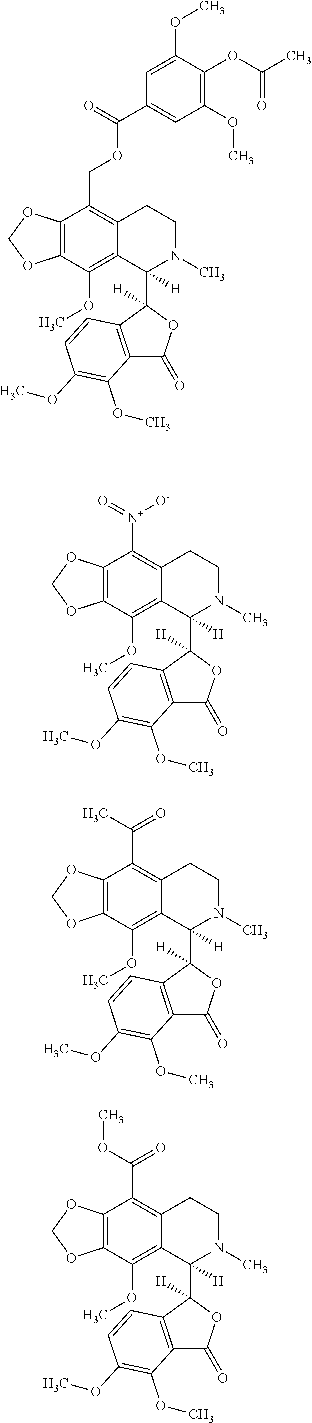

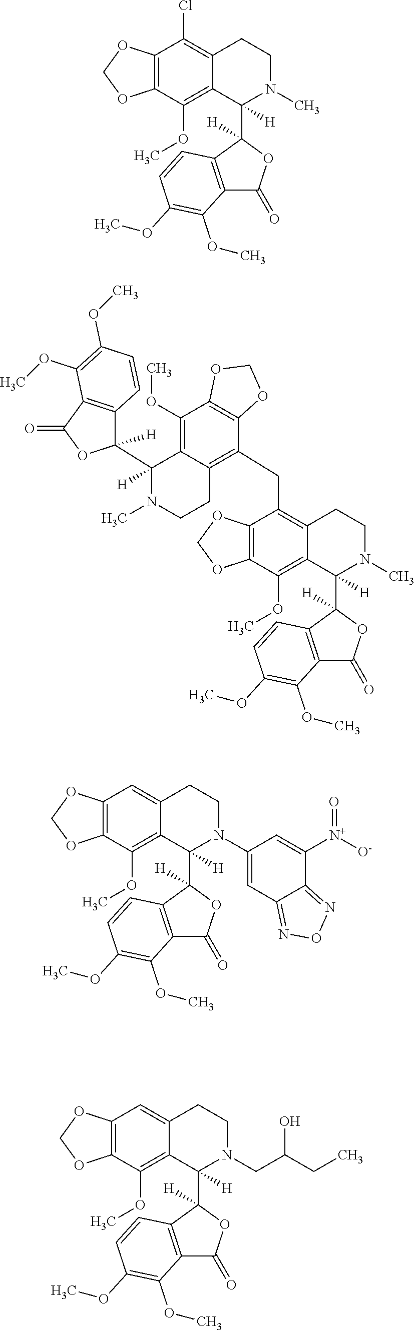

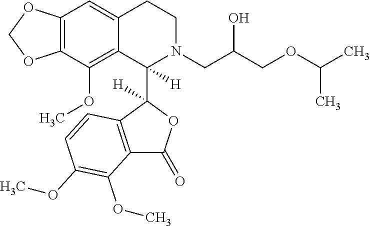

According to invention more preferred compounds are the derivatives (R,S)-noscapine of general formula 1.1:

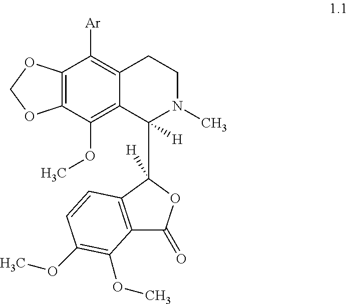

where: Ar is aryl or heteroaryl.

According to invention more preferred compounds are also derivatives (R,S)-noscapine of general formula 1.2:

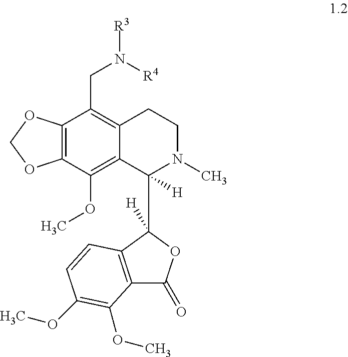

where: R3 and R4 independently of each other are the identical either different groups of the amino group, selected from hydrogen, alkyl, aryl, or R3 and R4 together with the atom of nitrogen, with which they are connected, they lock through R3 and R4 azaheterocycle.

According to the present invention, more preferred compounds are also derivatives of (R,S)-noscapine of general Formula 1.3: where: R3 and R4 have the values, indicated for the compounds of general formula 1.2.

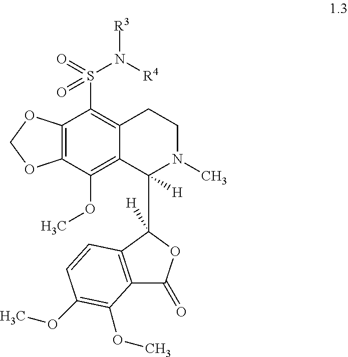

The most preferred compounds of general formula I are: 3-(9 iodo-4-methoxy-6-methyl-5,6,7,8-tetrahydro 1,3 dioxolo-4,5-g isoquinolin-5-yl)-6,7-dimethoxy-3H-isobenzofuran-1-on 1 (1), 3˜(4-methoxy-6-methyl-9-chloromethyl-5,6,7,8-tetrahydro 1,3-dioxolo 4,5-g isoquinoline-5-yl)-6,7-dimethoxy-3H-isobenzofuran-1-on 1 (2), 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carbaldehyde 1 (3), 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carboxylic acid 1 (4), 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carboxylic acid 1 (5), 3-(9 methoxymethyl-methoxy-6-methyl-5,6,7,8-tetrahydro-1,3-di-oxolo-4,5-g-isoquinolin-9-yl)-6,7-dimethoxy-3H-isobenzofuran-1-one 1 (6) and 5-(4,5-dimethoxy-3-oxo-1,3-dihydroisobenzofuran-1-yl)-4-methoxy-6-methyl-5, b, 7,8-tetrahydro-1,3 dioxolo 4,5-g isoquinoline-9-sulfonyl chloride; 1 (7):

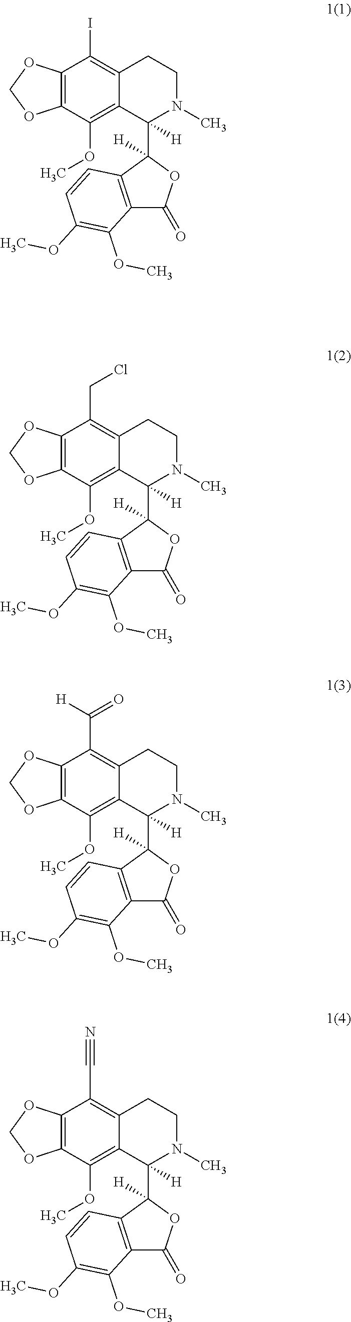

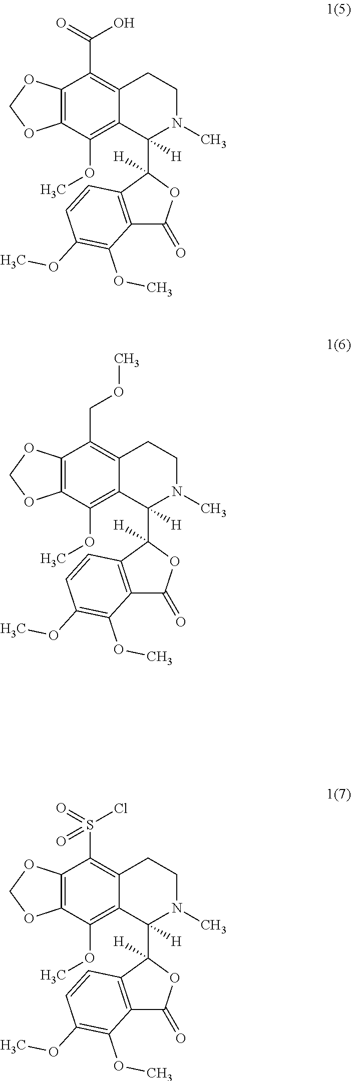

The most preferred compounds general formula 1.1 are: 3-(9 phenyl-4-methoxy-6-methyl-5,6,7,8-tetrahydro 1,3 dioxolo-4,5-g isoquinoline-5-yl)-b, 7 dimethoxy-3H-isobenzofuran-1-one 1.1 (1), 3-(9-p-tolyl-4-methoxy-6-methyl-5,6,7,8 tetrahydro 1,3 dioxolo-4,5-g isoquinoline-5-yl)-6,7-dimethoxy-3H-isobenzofuran-1-one 1.1 (2), 3-9(4-methoxyphenyl)-4-methoxy-6-methyl-5,6,7,8-tetrahydro-1,3 dioxolo-4,5-g isoquinoline-5-yl-6,7-dimethoxy-3H-isobenzofuran-1-on 1.1 (3), 3-9(4-chlorphenyl)-4-methoxy-6-methyl-5,6,7,8-tetrahydro-1, 3 dioxolo 4,5-g isoquinoline-5-yl-6,7-dimethoxy-3H-isobenzofuran-1-on 1.1 (4), 3-9 (4-trifluoromethylphenyl)-4-methoxy-6-methyl-5,6,7,8-tetra-hydro 1,3 dioxolo-4,5-g isoquinoline-5-yl-6,7-dimethoxy-3H-isoben-zofuran-1-on 1.1 (5), 3-9 (4-dimethylaminophenyl)-4-methoxy-6-methyl-5,6,7,8-tetra-hydro 1,3 dioxolo-4,5-g isoquinoline-5-yl-6,7-dimethoxy-3H-isobenzofuran-1-yl 1.1 (6), 3-9 (4-nitrophenyl)-4-methoxy-6-methyl-5,6,7,8-tetrahydro 1, 3 di-oxolo 4,5-g isoquinoline-5-yl-6,7-dimethoxy-3H-isobenzofuran-1-one 1.1 (7), 3-9 (4 ethoxycarbonylphenyl)-4-methoxy-6-methyl-5,6,7,8-tetra-hydro 1,3 dioxolo 4,5-g isoquinoline-5-yl-6,7-dimethoxy-3H-isobenzofuran-1-one 1.1 (8)

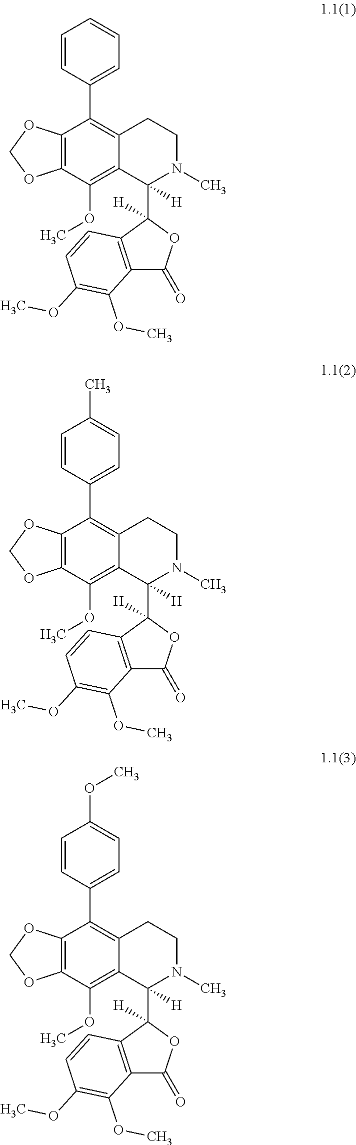

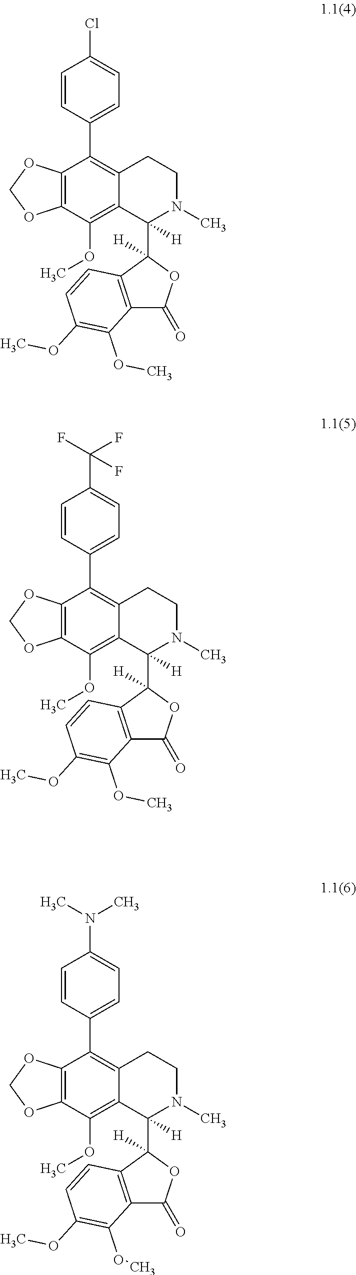

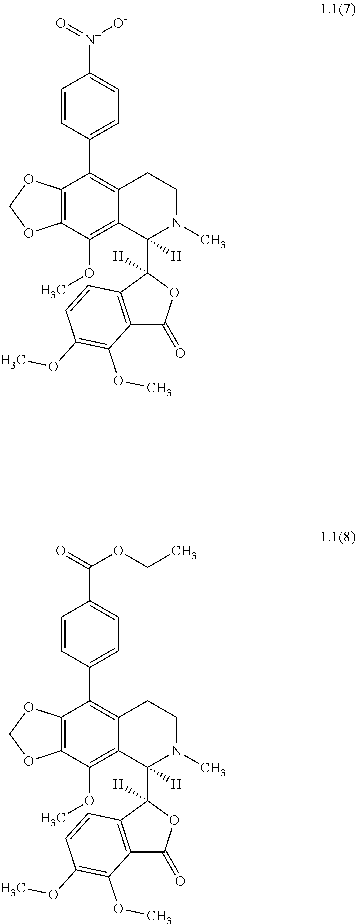

3-9(4-fluorophenyl)-4-methoxy-6-methyl-5,6,7,8-tetrahydro 1,3 dioxolo 4,5-g isoquinoline-5-yl-6,7-dimethoxy-3H-isobenzofuran-1-on 1.1 (9), 3-9-m-tolyl-4 methoxy-6-methyl-5,6,7,8-tetrahydro 1,3 dioxolo-4,5-g isoquinoline-5-yl-6,7-dimethoxy-3H-isobenzofuran-1-one 1.1 (10), 3-9 (3-methoxyphenyl)-4-methoxy-6-methyl-5,6,7,8-tetrahydro 1,3-dioxolo 4,5-g isoquinoline-5-yl-6,7-dimethoxy-3H-isobenzofuran-1-one 1.1 (11), 3-9 (3-chlorophenyl)-4-methoxy-6-methyl-5,6,7,8-tetrahydro 1,3-dioxolo 4,5-g isoquinoline-5-yl-6,7-dimethoxy-3H-isobenzofuran-1-yl 1.1 (12), 3-9 (3-fluorophenyl)-4-methoxy-6-methyl-5,6,7,8-tetrahydro 1, 3-dioxolo 4,5-g isoquinoline-5-yl-6,7-dimethoxy-3H-isobenzofuran-1-yl 1.1 (13), 3-9 (3-nitrophenyl)-4-methoxy-6-methyl-5,6,7,8-tetrahydro 1,3 dioxolo 4,5-g isoquinoline-5-yl-6,7-dimethoxy-3H-isobenzofuran-1-one 1.1 (14), 3-9 (3-trifluoromethylphenyl)-4-methoxy-6-methyl-5,6,7,8-tetra-hydro 1,3-dioxolo 4,5-g isoquinoline-5-yl-6,7-dimethoxy-3H-iso-benzofuran-1-one 1.1 (15), 3-9 (3,4-dimethylphenyl)-4-methoxy-6-methyl-5,6,7,8-tetrahydro-1,3-dioxolo 4,5-g isoquinoline-5-yl-6,7-dimethoxy-3H-isobenzofuran 1-yl 1.1 (16), 1.1 (13) 1.1 (14) 1.1 (15) 1.1 (16)

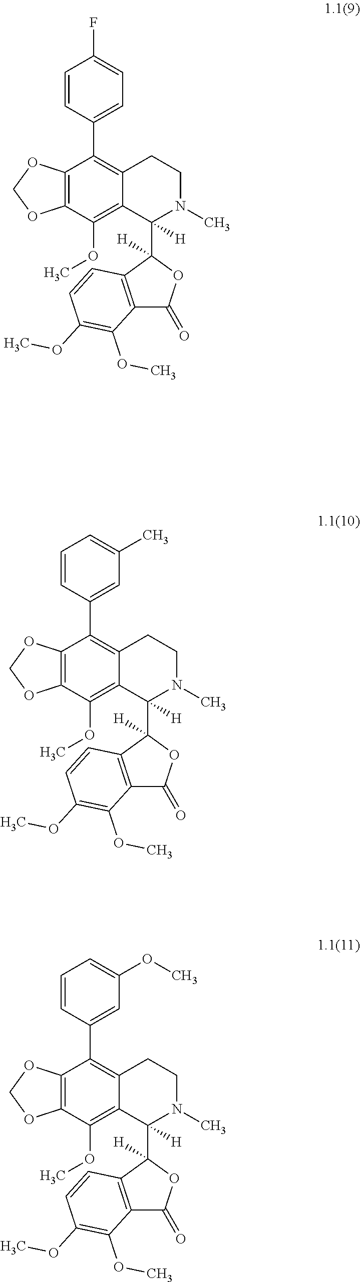

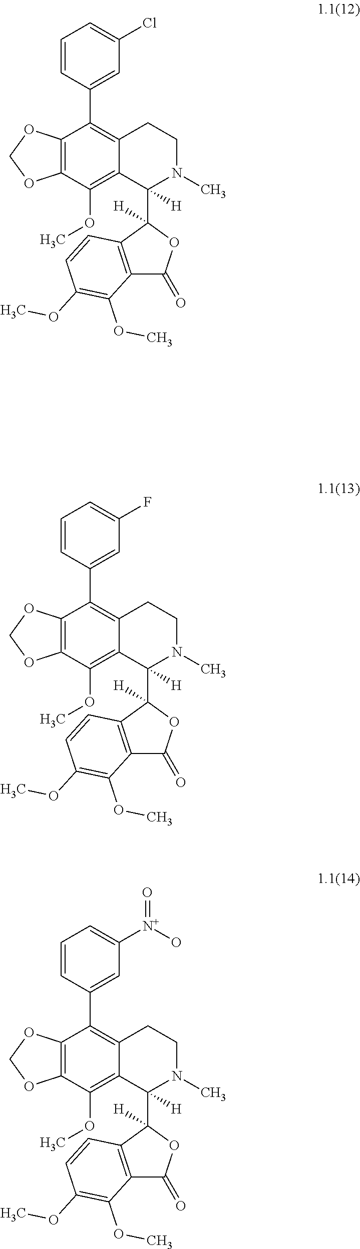

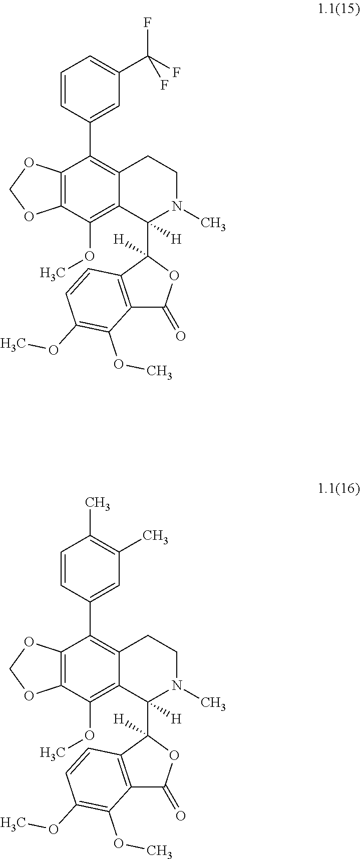

3-9 (3-pyridil)-4-methoxy-6-methyl-5,6,7,8-tetrahydro 1,3-dioxolo 4,5-g isoquinoline-5-yl-6,7-dimethoxy-3H-isobenzofuran-1-on 1.1 (17), 3-9(4-pyridyl)-4-methoxy-6-methyl-5,6,758-tetrahydro 1,5,3-dioxolo 4,5-g isoquinoline-5-yl-6,7-dimethoxy-3H-isobenzofuran-1-one 1.1 (18), 3-9 (2-pyridyl)-4-methoxy-6-methyl-5,6,7,8-tetrahydro 1,3-dioxolo 4,5-g isoquinoline-5-yl-6,7-dimethoxy-3H-isobenzofuran-1-one 1.1 (19), 3-9 (2-thienyl)-4-methoxy-6-methyl-5,6,7,8-tetrahydro 1,3-dioxolo 4,5-g isoquinoline-5-yl-6,7-dimethoxy-3H-isobenzofuran-1-on 1.1 (20), 3-9 (3-thenyl)-4-methoxy-6-methyl-5,6,7,8-tetrahydro 1,3-dioxolo 4,5-g isoquinoline-5-yl-6,7-dimethoxy-3H-isobenzofuran-1-on 1.1 (21), 3-9 (2-furyl)-4-methoxy-6-methyl-5,6,7,8-tetrahydro 1,3-di oxolo 4,5-g isoquinoline-5-yl-6,7-dimethoxy-3H-isobenzofuran-1-one 1.1 (22), 3-9 (5-indolyl)-4-methoxy-6-methyl-5,6,7,8-tetrahydro 1,3-di-oxolo 4,5-g isoquinoline-5-yl-6,7-dimethoxy-3H-isobenzofuran-1-on 1.1 (23), 3-9 (5-pyrimidinyl)-4-methoxy-b-methyl 5,6,7,8-tetrahydro 1,3-di-oxolo 4,5-g isoquinoline-5-yl-6,7-dimethoxy-3H-isobenzofuran-1-one 1.1 (24), 1.1 (21) 1.1 (22) 1.1 (23) 1.1 (24)

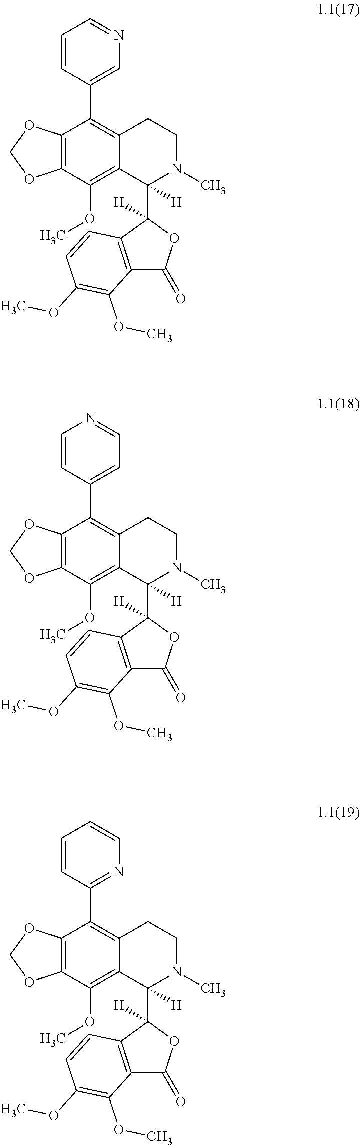

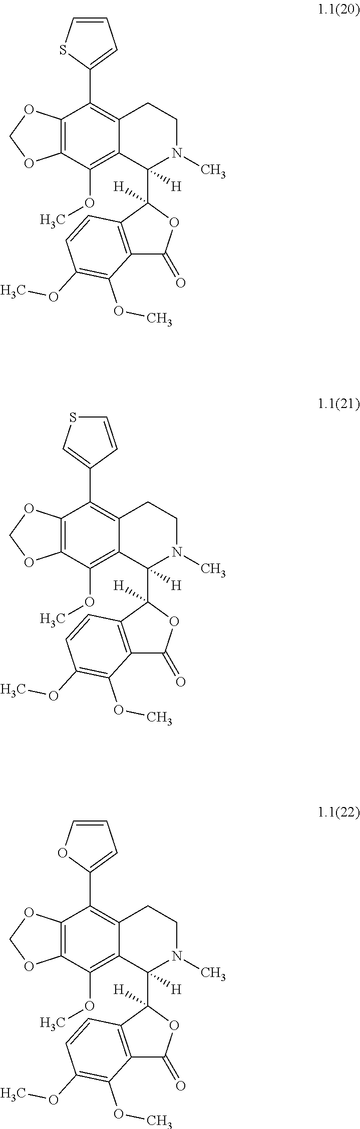

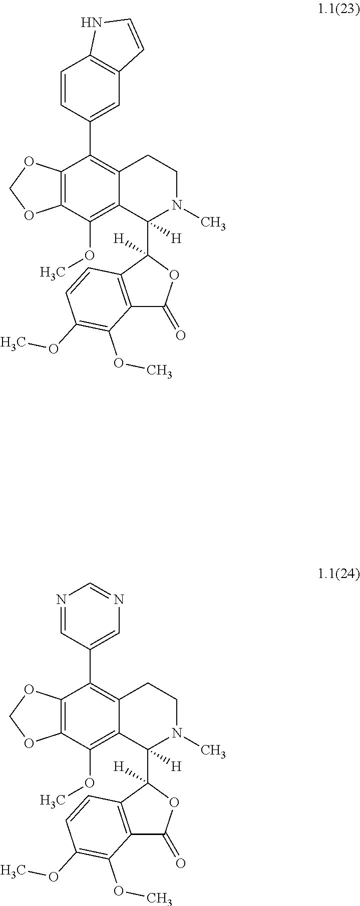

3-9 (2-benzofuranyl)-4-methoxy-b-methyl-5,6,7,8-tetrahydro 1,3-dioxolo 4,5-g isoquinoline-5-yl-6,7-dimethoxy-3H-isobenzofuran-1-yl 1.1 (25), 3-9 (3-dimethylaminophenyl)-4-methoxy-6-methyl-5,6,7,8-tetrahydro 1,3-di oxolo 4,5-g isoquinoline-5-yl-6,7-dimethoxy-3H-isobenzofuran-1-one 1.1 (26), 3-9 (6 methoxypyridine-3-yl)-4-methoxy-6-methyl-5,6,7,8-tetrahydro 1,3-di-oxolo 4,5-g isoquinoline-5-yl-6,7-dimethoxy-3H-isobenzofuran-1-one 1.1 (27), 3-9 (3-carboxyphenyl)-4-methoxy-6-methyl-5,6,7,8-tetrahydro 1,3-dioxolo 4,5-g isoquinoline-5-yl-6,7-dimethoxy-3H-isobenzofuran-1-one 1.1 (28), 3-9 (4-carboxyphenyl)-4-methoxy-6-methyl-5,6,7,8-tetrahydro 1,3-di oxolo 4,5-g isoquinoline-5-yl-6,7-dimethoxy-3H-isobenzofuran-1-one 1.1 (29), 3-9 (3-carbamoylphenyl)-4-methoxy-6-methyl-5,6,7,8-tetrahydro 1,3-di-oxolo 4,5-g isoquinoline-5-yl-6,7-dimethoxy-3H-isobenzofuran-1-yl 1.1 (30), 3-9 (4-isoquinolineyl)-4-methoxy-6-methyl-5,6,7,8-tetrahydro 1,3-di-oxolo 4,5-g isoquinoline-5-yl-6,7-dimethoxy-3H-isobenzofuran-1-one 1.1 (31), 3-9 (4 pyridinyl)-4-methoxy-6-methyl-5,6,7,8-tetrahydro 1,3-dioxolo 4,5-g isoquinoline-5-silt-6,7-dimethoxy-3H-isobenzofuran-1-yl 1.1 (32), 1.1 (29) 1.1 (30) 1.1 (31) 1.1 (32) 3-9

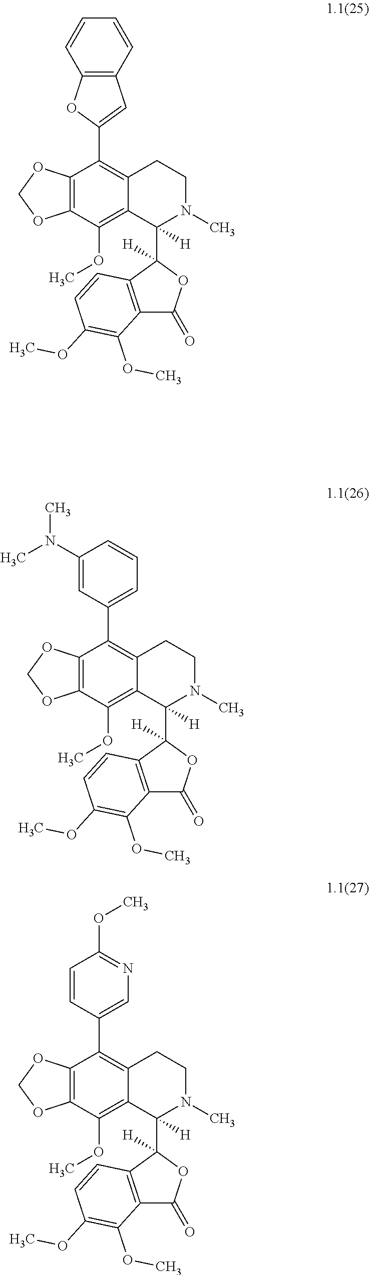

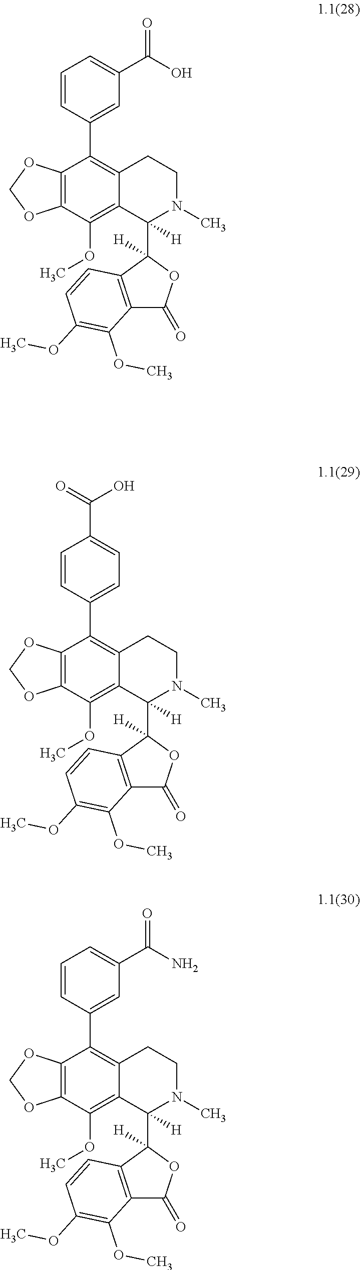

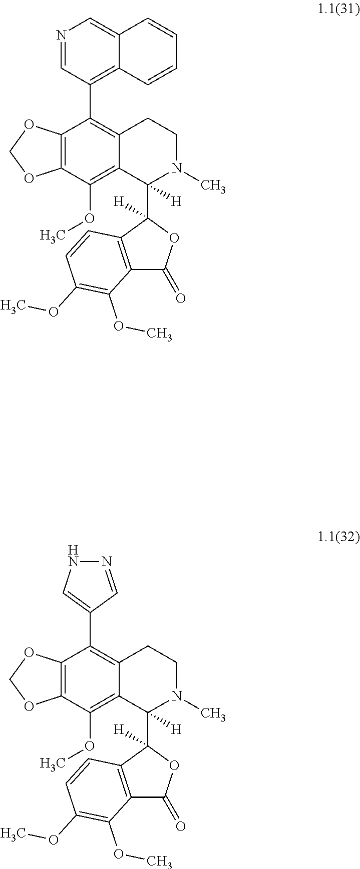

(1-tert-butyloxycarbonylindol-2-yl-4-methoxy-6-methyl-5,6,7,8-tetrahydro 1,3-di-oxolo 4,5-g isoquinoline-5-yl-6,7-dimethoxy-3H-isobenzofuran-1-on 1.1 (33), 3-(1 tert-butoxycarbonyl-5-methoxyindol-2-yl-4-methoxy-b-methyl-5,6,7,8 tetrahydro 1,3-di-oxolo 4,5-g isoquinoline-5-yl-b, 7-dimethoxy-3H-isobenzofuran-1 it 1.1 (34), 3-9 (3-hydroxyphenyl)-4-methoxy-6-methyl-5,6,7,8-tetrahydro 1,3-di-oxolo 4,5-g isoquinoline-5-yl-6,7-dimethoxy-3H-iodo-benzofuran-1-on 1.1 (35), 3-9 (4 hydroxyphenyl)-4-methoxy-6-methyl-5,6,7,8-tetrahydro 1,3-di-oxolo 4,5-g isoquinoline-5-yl-6,7-dimethoxy-3H-isobenzofuran-1-yl 1.1 (36), 3-9 (4-metansulfonylphenyl)-4-methoxy-6-methyl-5,6,7,8-tetrahydro 1,3-di-oxolo 4,5-g isoquinoline-5-yl-6,7-dimethoxy-3H-isobenzofuran-1-on 1.1 (37), 3-9 (3-thenyl)-4-methoxy-6-methyl-5,6,7,8-tetrahydro 1,3-di-oxolo 455-g isoquinoline-5-yl-b, 7-dimethoxy-3H-isobenzofuran-1 it 1.1 (38), 3-9 (5-indazolyl)-4-methoxy-6-methyl-5,6,7,8-tetrahydro 1,3-di-oxolo 4,5-g isoquinoline-5-yl-6,7-dimethoxy-3H-isobenzofuran-1-on 1.1 (39), 1{4-5-(4,5-dimethoxy-3-oxo-1,3-dihydro-isobenzofuran-1-yl)-4-methoxy-6-methyl-5, b, 7,8-tetrahydro-1,3 dioxolo 4,5-isoquinoline-9-yl-phenyl}-3-phenyl-mochevina 1.1 (40) or 1{3-5-(4,5-dimethoxy-3-oxo-1,3-dihydro-isobenzofuran-1-yl)-4-methoxy-b-methyl-5,6,7,8 tetrahydro-1, 3 dioxolo 4,5-isoquinoline-9-yl-phenyl}-3-phenyl-urea 1.1 (41).

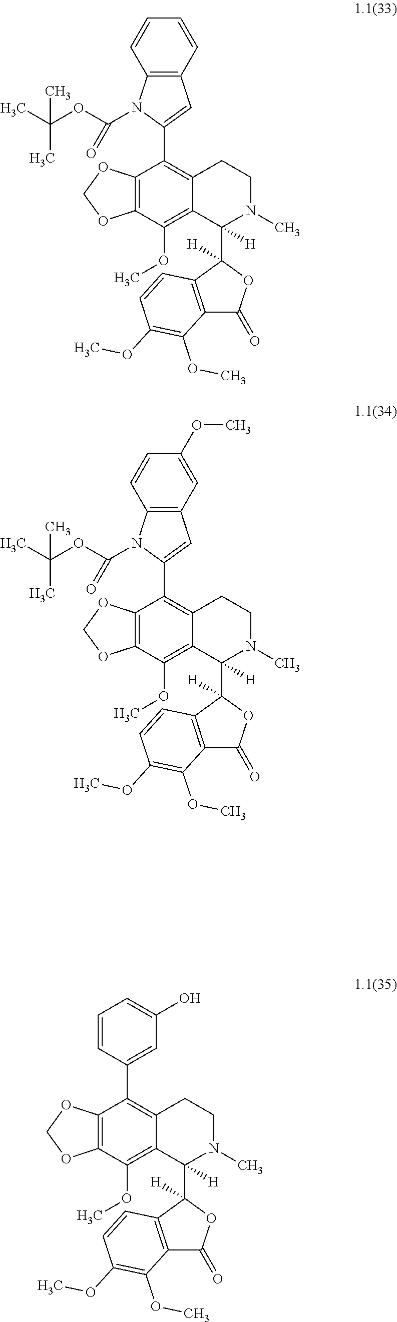

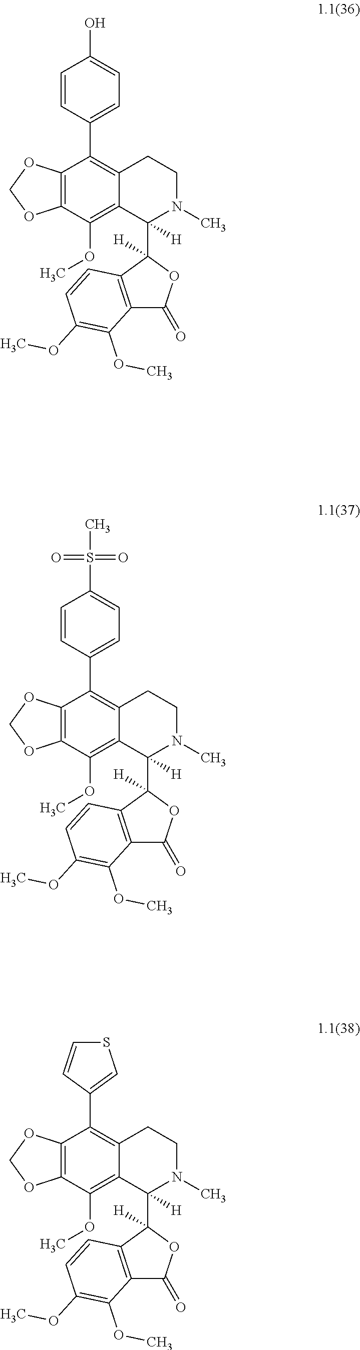

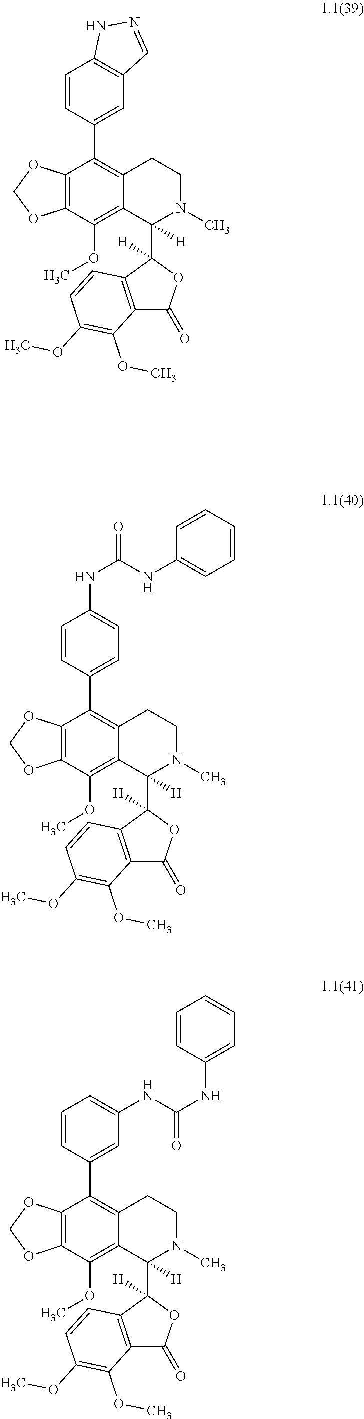

The most preferred compounds of general formula 1.2 are: 3-(9 benzylaminomethyl-4-methoxy-6-methyl-5,6,7,8-tetra-hydro 1,3 dioxolo 4,5-g isoquinoline-5-yl)-6,7-dimethoxy-3H-isobenzofuran-1-yl 1.2 (1), 3-(9 diethylaminomethyl-4-methoxy-6-methyl-5,6,7,8-tetrahydro-1,3 dioxolo 4,5-g isoquinoline-5-yl)-6,7-dimethoxy-3H-isobenzofuran 1-on 1.2 (2), 3-(9-N pyrrolidinomethyl-4-methoxy-6-methyl-5,6,7,8-tetrahydro-1,3 dioxolo 4,5-g isoquinoline-5-yl)-6,7-dimethoxy-3H-isobenzofuran-1-on 1.2 (3), 3-(9-N piperidinomethyl-4-methoxy-6-methyl-5,6,7,8-tetrahydro-1,3 dioxolo 4,5-g isoquinoline-5-yl)-6,7-dimethoxy-3H-isobenzofuran-1-on 1.2 (4), 3-(9-N morpholinomethyl-4-methoxy-6-methyl-5,6,7,8-tetrahydro-1,3 dioxolo 4,5-g isoquinoline-5-yl)-6,7-dimethoxy-3H-isobenzofuran-1-on 1.2 (5), 3-(9-N-piperazinomethyl-4-methoxy 6-methyl-5,6,7,8-tetrahydro-1,3 dioxolo 4,5-g isoquinoline-5-yl)-6,7-dimethoxy-3H-isobenzo-furan-1-on 1.2 (6), 3-(9-aminomethyl-4-methoxy-6-methyl-5,6,7,8-tetrahydro-1,3 dioxolo 4,5-g isoquinoline-5-yl)-6,7-dimethoxy-3H-isobenzofuran-1-on 1.2 (7). 1.2 (1) 1.2 (2) 1.2 (3) 1.2 (4)

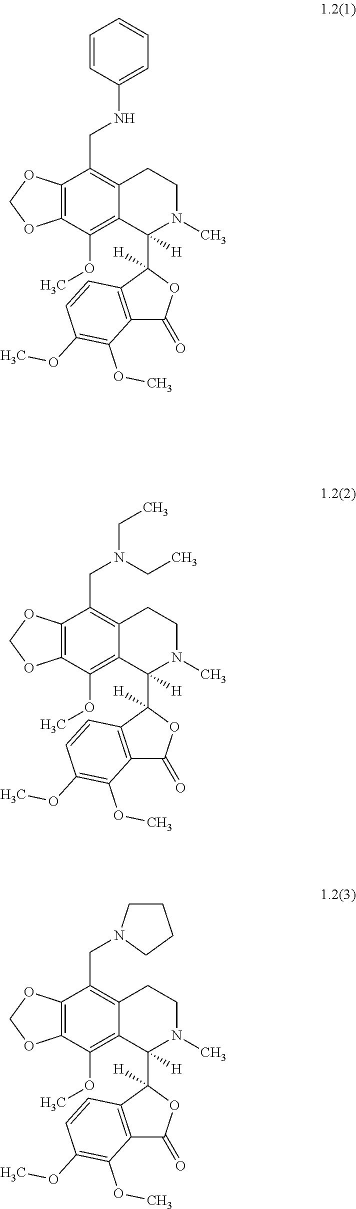

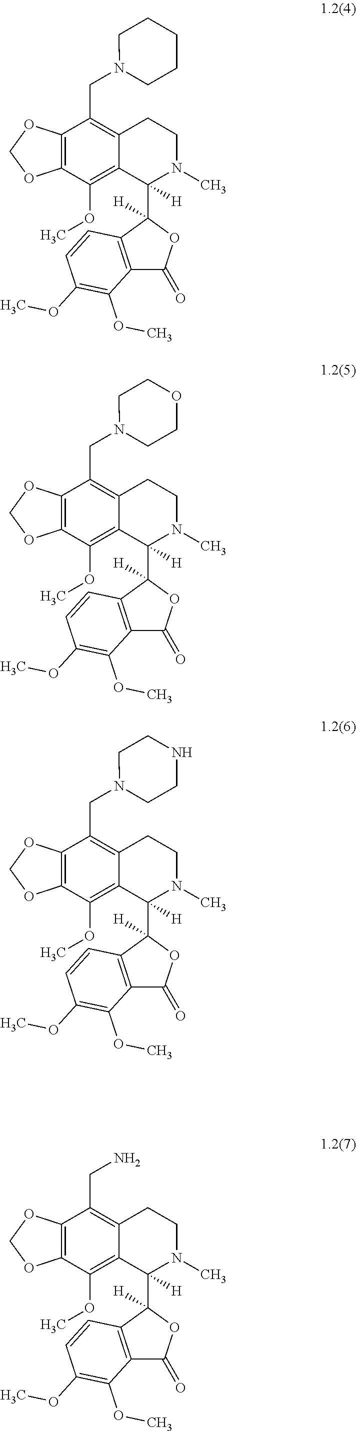

The most preferred compounds general formula 1.3 are: is 5th (4,5-dimethoxy-3-oxo-1,3-dihydroisobenzofuran-1-yl)-4-methoxy-6-methyl-5,6,7,8-tetrahydro-1, 3 dioxolo 4,5-g isoquinoline-9-sulfonylamid 1.3 (1), 6,7-dimethoxy-3-4 methoxy-6-methyl-9-(pyrrolidin-1-sulfonyl)-5,6,7,8-tetrahydro-1,3 dioxolo 4,5-g isoquinoline-5-yl-3H-isobenzofuran-1-on 1.3 (2), 6,7-dimethoxy-3-4-methoxy-6-methyl-9 (piperidin-1-sulfonyl)-5,6,7,8-tetrahydro-1,3 dioxolo 4,5-g isoquinoline-5-yl-ZN-isobenzofuran-1-yl 1.3 (3), 6,7-dimethoxy-Z-4-methoxy-6-methyl-9-(morpholin-yl sulfonyl)-5,6,7,8-tetrahydro-1,3 dioxolo 4,5-g isoquinoline-5-yl-3H-isobenzofuran-1-on 1.3 (4), 6,7-dimethoxy-3-4-methoxy-6-methyl-9-(piperazin-1-sulfonyl)-5,6,7,8-tetrahydro-1,3 dioxolo 4,5-g isoquinoline-5-yl-3H-isobenzofuran-1-on 1.3 (5), 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-sulfonyl diethyl acid 1.3 (6), 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-sulfonyl di(2-hydroxyethyl)amide 1.3 (7). 1.3 (5) 1.3 (6) 1.3 (7)

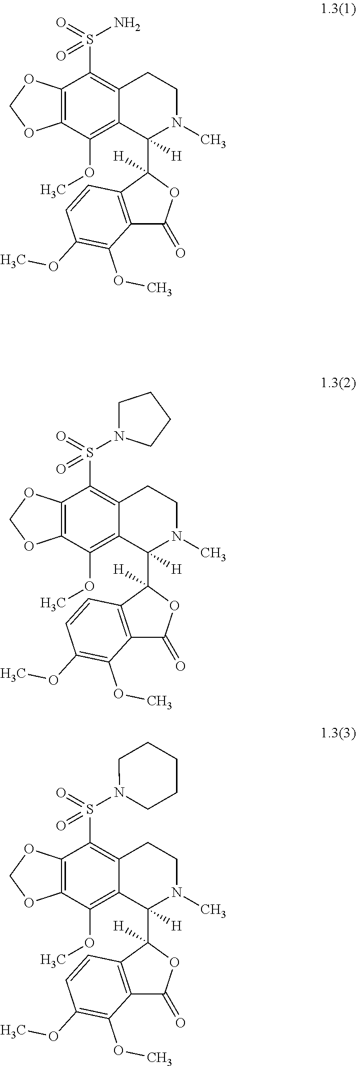

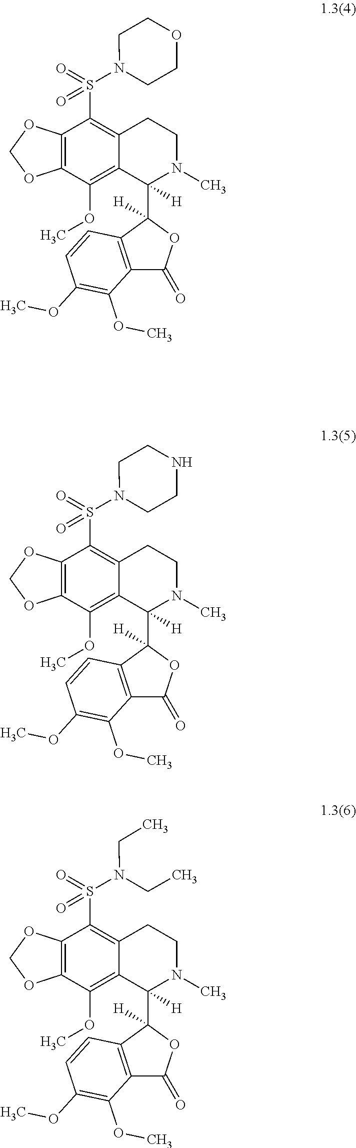

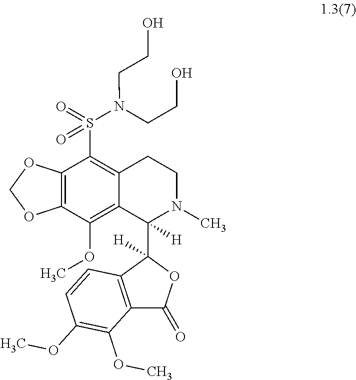

### Pharmaceutically-Acceptable Salts

Examples of suitable pharmaceutically acceptable salts include inorganic acid addition salts such as sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with an acidic amino acid such as aspartate and glutamate; alkali metal salts such as sodium and potassium; alkaline earth metal salts such as magnesium and calcium; ammonium salt; organic basic salts such as trimethylamine, triethylamine, pyridine, picoline, dicyclohexylamine, and N,N′-dibenzylethylenediamine; and salts with a basic amino acid such as lysine and arginine. The salts can be in some cases hydrates or ethanol solvates. The stoichiometry of the salt will vary with the nature of the components.

Pharmaceutically acceptable salts may be obtained using standard procedures well known in the art, for example by reacting the amine group with a suitable acid affording a physiologically acceptable anion. In one embodiment, the salt is a hydrochloride salt of the compound.

Prodrugs and Derivatives

The active compound can be administered as any salt or prodrug that upon administration to the recipient is capable of providing directly or indirectly the parent compound, or that exhibits activity itself.

Non-limiting examples include forms of 9-amino-noscapine in which the amine group has been alkylated, acylated, or otherwise modified (a type of “pharmaceutically acceptable prodrug”).

Further, the modifications can affect the biological activity of the compound, in some cases increasing the activity over the parent compound. This can easily be assessed by preparing the salt or prodrug and testing its antimicrobial or other activity according to the methods described herein, or other methods known to those skilled in the art.

Prodrug forms of the compound include the following types of derivatives where each R group individually can be hydrogen, substituted or unsubstituted alkyl, aryl, alkenyl, alkynyl, heterocycle, alkylaryl, aralkyl, aralkenyl, aralkynl, cycloalkyl or cycloalkenyl groups.

(a) Carboxamides, —NHC(O)R

(b) Carbamates, —NHC(O)OR

(c) (Acyloxy)alkyl Carbamates, NHC(O)OROC(O)R

(d) Enamines, —NHCR(═CHCO₂R) or —NHCR(═CHCONR₂)

(e) Schiff Bases, —N═CR₂

(f) Mannich Bases (from carboximide compounds), RCONHCH₂NR₂

As used herein, alkyl refers to C₁₋₈ straight, branched, or cyclic alkyl groups, and alkenyl and alkynyl refers to C₂₋₈ straight, branched or cyclic moieties that include a double or triple bond, respectively. Aryl groups include C₆₋₁₀ aryl moieties, specifically including benzene. Heterocyclic groups include C₃₋₁₀ rings which include one or more O, N, or S atoms. Alkylaryl groups are alkyl groups with an aryl moiety, and the linkage to the nitrogen at the 9-position on the noscapine framework is through a position on the alkyl group. Arylalkyl groups are aryl groups with an alkyl moiety, and the linkage to the nitrogen at the 9-position on the noscapine framework is through a position on the aryl group. Aralkenyl and aralkynyl groups are similar to aralkyl groups, except that instead of an alkyl moiety, these include an alkenyl or alkynyl moiety. Substituents for each of these moieties include halo, nitro, amine, thio, hydroxy, ester, thioester, ether, aryl, alkyl, carboxy, amide, azo, and sulfonyl.

Other prodrugs include prodrugs that are converted in biological milieu via ester hydrolysis via an enzymatic route rather than chemical hydrolysis, for example, by serine-dependent esterases. Representative prodrugs of this type are described, for example, in Amsberry et al., “Amine Prodrugs Which Utilize Hydroxy Amide Lactonization. II. A Potential Esterase-Sensitive Amide Prodrug,” Pharmaceutical Research, Volume 8(4): 455-461(7) (April 1991).

Azo-based prodrugs can also be used. For example, bacterial reductases can use reductive cleavage to convert the following azo prodrug in vivo to the active form.

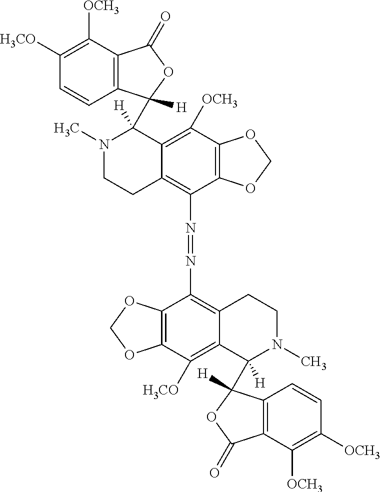

### II. Methods of Preparing the Compounds

The compounds can be prepared by performing electrophilic aromatic substitution on the isoquinoline ring of noscapine, typically under conditions that do not result in significant hydrolysis of the noscapine framework. The substituents typically are added to the 9-position on the isoquinoline ring, although yields can be optimized and by-products may be present and need to be removed during a purification step. More optimized syntheses of representative compounds, such as 9-nitro-nos, 9-iodo-nos, 9-bromo-nos, and 9-iodo-nos, are provided in the Examples section.

Briefly, the nitration of the isoquinoline ring in noscapine can be accomplished by using stoichiometric silver nitrate and a slight excess of trifluoroacetic anhydride.

The halogenation of noscapine involved various procedures, which varied depending on the particular halogen, as summarized below in Scheme 1.

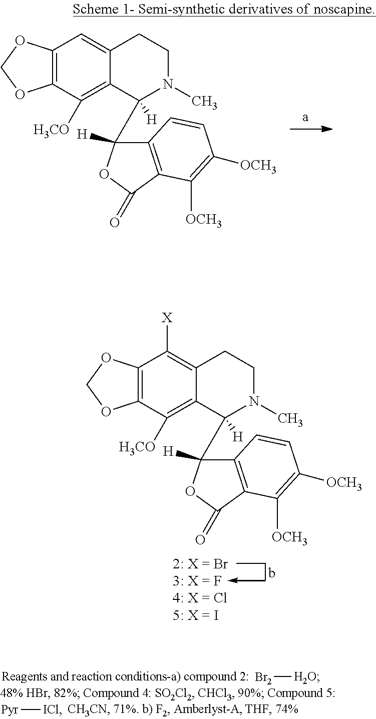

Noscapine can be brominated at the 9-position by reacting noscapine with concentrated hydrobromic acid. Noscapine can be fluorinated using the fluoride form of Amberlyst-A 26, or by Br/F exchange. Iodination of noscapine typically required low-acid conditions. One successful approach for preparing 9-I-nos involved treating a solution of noscapine in acetonitrile with pyridine-iodine chloride at room temperature for 6 hours followed by raising the temperature to 100° C. for another 6 hours.

9-Chloro-Nos can be prepared by performing electrophilic aromatic substitution on the isoquinoline ring of noscapine, typically under conditions that do not result in significant hydrolysis of the noscapine framework. The chloro substituent can be added to the 9-position on the isoquinoline ring using a variety of known aromatic chlorination conditions, although yields can be optimized and by-products may be present and need to be removed during a purification step. More optimized syntheses are provided in the Examples section.

The halogenation of noscapine involved various procedures, which varied depending on the particular halogen, as summarized below in Scheme 1.

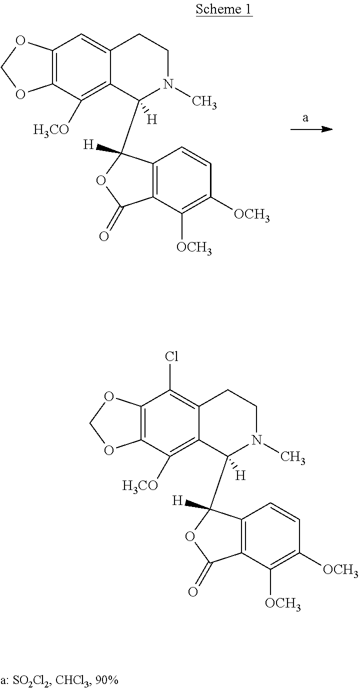

Chlorination of noscapine using sulfuryl chloride in chloroform at low temperature gave excellent yields and the desired regioselectivity.

9-Amino-Nos can be prepared, for example, by first performing a nitration reaction on the isoquinoline ring of noscapine, ideally under conditions that do not result in significant hydrolysis of the noscapine framework. The nitro group adds predominantly at the 9-position of noscapine. The nitro group can then be reduced to an amino (NH₂) substituent using conventional techniques. Although yields can be optimized and by-products may be present and need to be removed during a purification step, the general synthetic strategy is shown below in Scheme I. More optimized syntheses are provided in the Examples section.

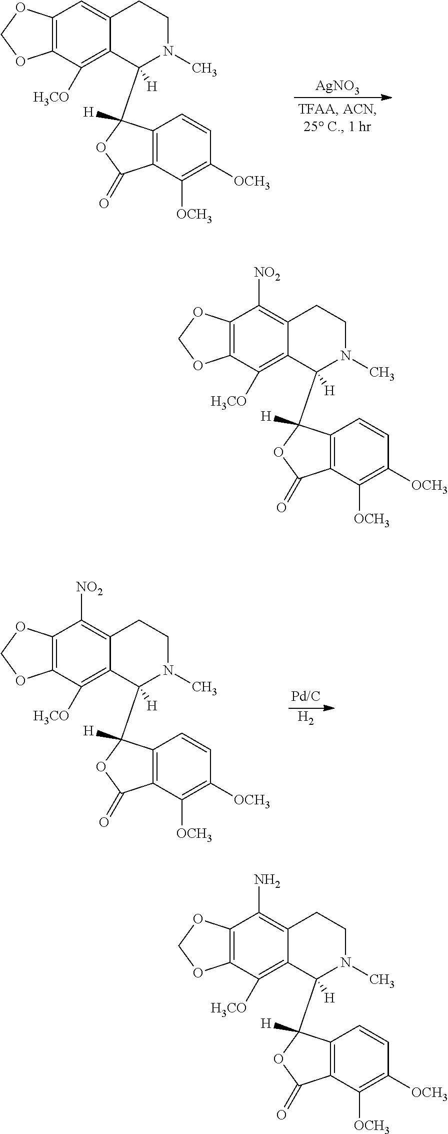

Other methods for reducing nitrates to amines are well known to those of skill in the art. Ideally, methods do not involve reagents which reduce or hydrolyze the lactone moiety. In some embodiments, the lactone can be protected with a suitable protecting group, the nitro group reduced to an amine, and the lactone deprotected.

In other embodiments, the nitro group can be converted to a diazonium salt, followed by displacement to form the amine.

Other amines than 9-NH₂ can be formed, for example, by first forming the 9-noscapine, and then converting the 9-NH₂ group to another moiety using alkylation reagents in alkylation reactions. Suitable alkylation reagents as are known in the art, and include C₁₋₈ alkyl halides, such as alkyl bromides and iodides.

Those skilled in the art that incorporation of other substituents onto the 9-position of the isoquinoline ring, and other positions in the noscapine framework, can be readily realized. Such substituents can provide useful properties in and of themselves or serve as a handle for further synthetic elaboration.

A number of other analogs, bearing substituents in the 9 position of the isoquinoline ring, can be synthesized from the corresponding amino compounds, via a 9-diazonium salt intermediate. The diazonium intermediate can be prepared, using known chemistry, by reduction of the 9-nitro compound to the 9-nitro amine compound, followed by reaction with a nitrite salt, typically in the presence of an acid. Examples of other 9-substituted analogs that can be produced from 9-diazonium salt intermediates include, but are not limited to: 9-hydroxy, 9-alkoxy, 9-fluoro, 9-chloro, 9-iodo, 9-cyano, and 9-mercapto. These compounds can be synthesized using the general techniques set forth in Zwart et al., supra. For example, the 9-hydroxy-noscapine analogue can be prepared from the reaction of the corresponding 9-diazonium salt intermediate with water. Likewise, 9-alkoxy noscapine analogues can be made from the reaction of the 9-diazonium salt with alcohols. Appropriate 9-diazonium salts can be used to synthesize cyano or halo compounds, as will be known to those skilled in the art. 9-Mercapto substitutions can be obtained using techniques described in Hoffman et al., J. Med. Chem. 36: 953 (1993). The 9-mercaptan so generated can, in turn, be converted to a 9-alkylthio substitutuent by reaction with sodium hydride and an appropriate alkyl bromide. Subsequent oxidation would then provide a sulfone. 9-Acylamido analogs of the aforementioned compounds can be prepared by reaction of the corresponding 9-amino compounds with an appropriate acid anhydride or acid chloride using techniques known to those skilled in the art of organic synthesis.

9-Hydroxy-substituted analogs of the aforementioned compounds can be used to prepare corresponding 9-alkanoyloxy-substituted compounds by reaction with the appropriate acid, acid chloride, or acid anhydride. Likewise, the 9-hydroxy compounds are precursors of both the 9-aryloxy and 9-heteroaryloxy via nucleophilic aromatic substitution at electron deficient aromatic rings. Such chemistry is well known to those skilled in the art of organic synthesis. Ether derivatives can also be prepared from the 9-hydroxy compounds by alkylation with alkyl halides and a suitable base or via Mitsunobu chemistry, in which a trialkyl- or triarylphosphine and diethyl azodicarboxylate are typically used. See Hughes, Org. React. (N.Y.) 42: 335 (1992) and Hughes, Org. Prep. Proced. Int. 28: 127 (1996) for typical Mitsunobu conditions.

9-Cyano-substituted analogs of the aforementioned compounds can be hydrolyzed to afford the corresponding 9-carboxamido-substituted compounds. Further hydrolysis results in formation of the corresponding 9-carboxylic acid-substituted analogs. Reduction of the 9-cyano-substituted analogs with lithium aluminum hydride yields the corresponding 9-aminomethyl analogs. 9-Acyl-substituted analogs can be prepared from corresponding 9-carboxylic acid-substituted analogs by reaction with an appropriate alkyllithium using techniques known to those skilled in the art of organic synthesis.

9-Carboxylic acid-substituted analogs of the aforementioned compounds can be converted to the corresponding esters by reaction with an appropriate alcohol and acid catalyst. Compounds with an ester group at the 9-pyridyl position can be reduced with sodium borohydride or lithium aluminum hydride to produce the corresponding 9-hydroxymethyl-substituted analogs. These analogs in turn can be converted to compounds bearing an ether moiety at the 9-pyridyl position by reaction with sodium hydride and an appropriate alkyl halide, using conventional techniques. Alternatively, the 9-hydroxymethyl-substituted analogs can be reacted with tosyl chloride to provide the corresponding 9-tosyloxymethyl analogs. The 9-carboxylic acid-substituted analogs can also be converted to the corresponding 9-alkylaminoacyl analogs by sequential treatment with thionyl chloride and an appropriate alkylamine. Certain of these amides are known to readily undergo nucleophilic acyl substitution to produce ketones.

9-Hydroxy-substituted analogs can be used to prepare 9-N-alkyl- or 9-N-arylcarbamoyloxy-substituted compounds by reaction with N-alkyl- or N-arylisocyanates. 9-Amino-substituted analogs can be used to prepare 9-alkoxycarboxamido-substituted compounds and 9-urea derivatives by reaction with alkyl chloroformate esters and N-alkyl- or N-arylisocyanates, respectively, using techniques known to those skilled in the art of organic synthesis.

Other possible synthetic methods involve nitrating the aromatic ring, and reducing the nitrate group to an amine group. Such nitration and reduction reactions are well known to those of skill in the art. Ideally, methods do not involve reagents which reduce or hydrolyze the lactone moiety. In some embodiments, the lactone can be protected with a suitable protecting group, the nitro group reduced to an amine, and the lactone deprotected.

In other embodiments, the nitro group can be converted to a diazonium salt, followed by displacement to form the amine.

Other amines than 9-NH₂ can be formed, for example, by first forming the 9-noscapine, and then converting the 9-NH₂ group to another moiety using alkylation reagents in alkylation reactions. Suitable alkylation reagents as are known in the art, and include C₁₋₈ alkyl halides, such as alkyl bromides and iodides.

The compounds of Formula V can be prepared as follows:

The methods make it possible to preserve the optical activity, inherent in the initial alkaloid. According to this invention is developed the method of obtaining 3-(9-iodo-4-methoxy-b-methyl-5,657,8-tetrahydro 1,3 dioxolo-4,5-g isoquinoline-5-yl)-6,7-dimethoxy-3H-isobenzofuran-1-one 1 (1), being consisted in action of ICl on (R,S)-noscapine (NSC) on acetic acid according to the following diagram:

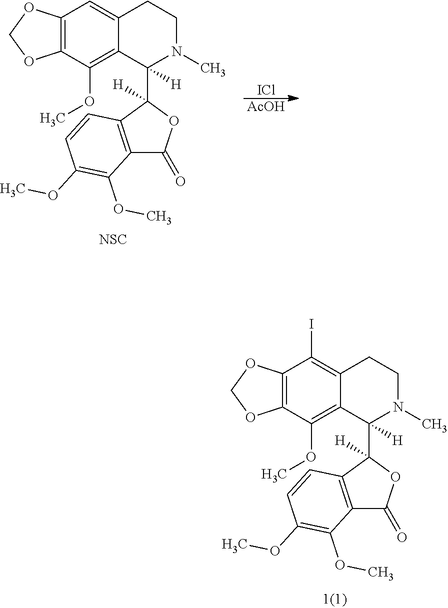

According to this invention is developed the method of obtaining 3-(9-chloromethyl-4-methoxy-6-methyl-5,6,7,8-tetrahydro 1,3-dioxolo 4,5-g isoquinoline-5-yl)-6,7-dimethoxy-3H-isobenzofuran-1-one 1 (2), that is consisted in action of thionyl chloride on 3-(9-hydroxymethyl-4-methoxy-6-methyl-5,6,7,8-tetrahydro 1,3-dioxolo 4,5-g isoquinoline-5-yl)-6,7-dimethoxy-3H-isobenzofuran-1-on A-04 according to the following diagram:

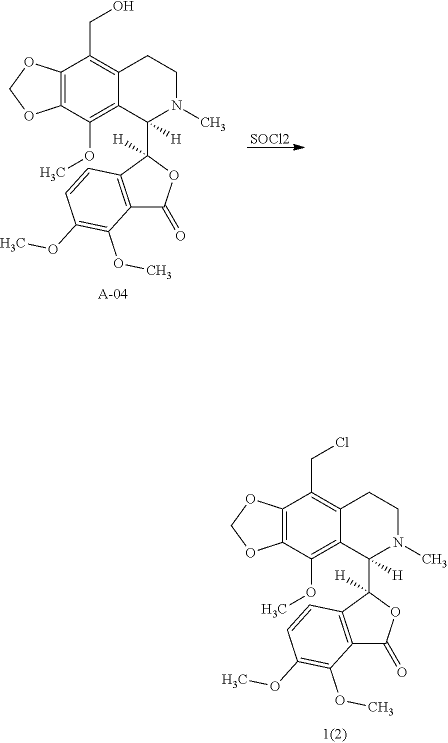

According to this invention is developed the method of obtaining 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carbaldehyde 1 (3), that is consisted in action of hexamethylentetramine 2 on 3-(9-chloromethyl-4-methoxy-6-methyl-5,6,7,8-tetraridpo 1,3-dioxolo 4,5-g isoquinoline-5-yl)-b, 7-dimethoxy-3H-isobenzofuran-1-one 1 (2) on organic solvent according to the following diagram:

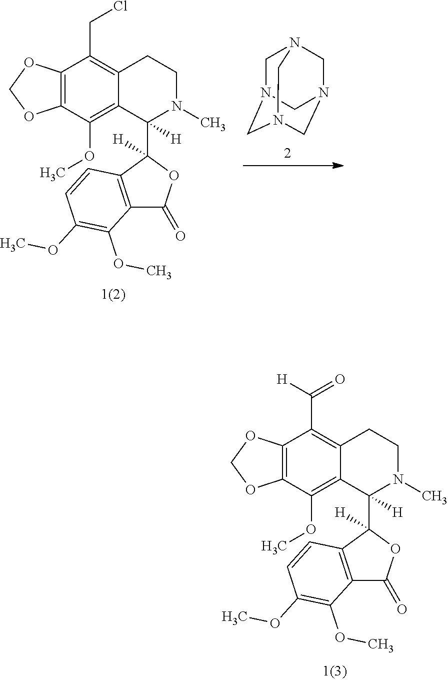

According to this invention is developed the method of obtaining 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carboxylic1 (4), that is consisted in action of cyanide of copper (the I) on 3-(9-Fromethoxy-6-methyl-5,6,7,8-tetrahydro 1, 3 dioxolo-4,5-g isoquinoline-5-yl)-6,7-dimethoxy-3H-isobenzofuran-1-one-ol or 9-iodo-4-methoxy-6-methyl-5,6,7,8-tetrahydro 1,3 dioxolo-4,5-g isoquinoline-5-yl)-6,7-dimethoxy-3H-isobenzofuran-1-on 1 (1) on aprotic solvent according to the following diagram:

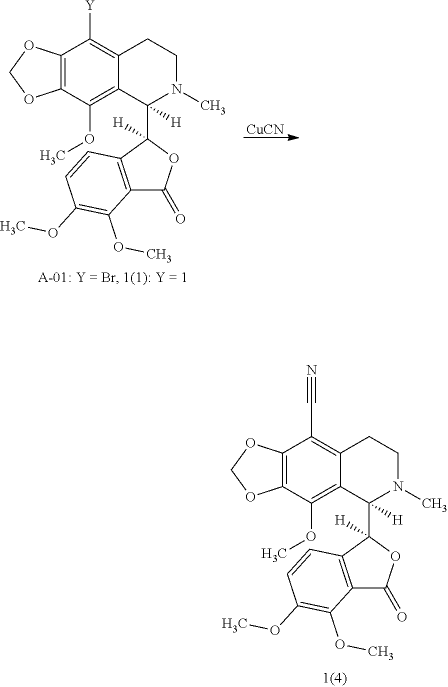

According to this invention is developed the method of obtaining 5-(4,5-dimethoxy-3-oxo-1,3-dihydroisobenzofuran-1-yl)-4-methoxy-6-methyl-5, b, 7,8-tetrahydro-1,3 dioxolo 4,5-g isoquinoline-9-carboxylic acid 1 (5) by hydrolysis of 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-nitrile 1 (4) according to the following diagram:

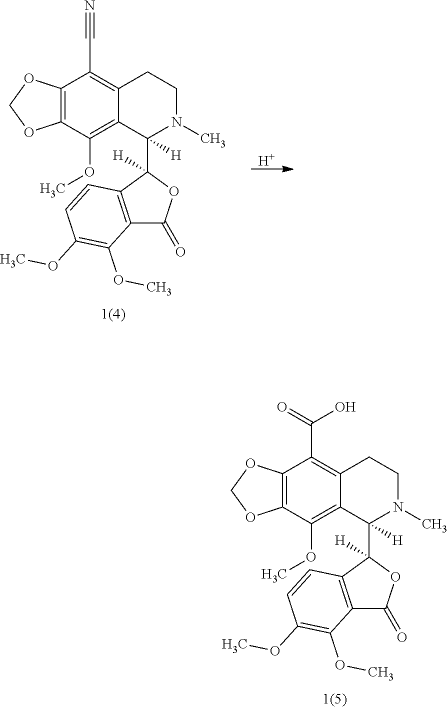

According to this invention is developed the method of obtaining 3-(9-methoxymethyl-4-methoxy-6-methyl-5,6,7,8-tetrahydro 1,3-dioxolo 4,5-g isoquinoline-5-yl)-6,7-dimethoxy-3H-isobenzofuran-1-on 1 (6) by reaction of 3-(9-chloromethyl-4-methoxy-6-methyl-5,6,7,8-tetrahydro 1,3-di-oxolo 4,5-g isoquinoline-5-yl)-6,7-dimethoxy-3H-isobenzofuran-1-one 1 (2) with methanol in the presence of base according to the following diagram:

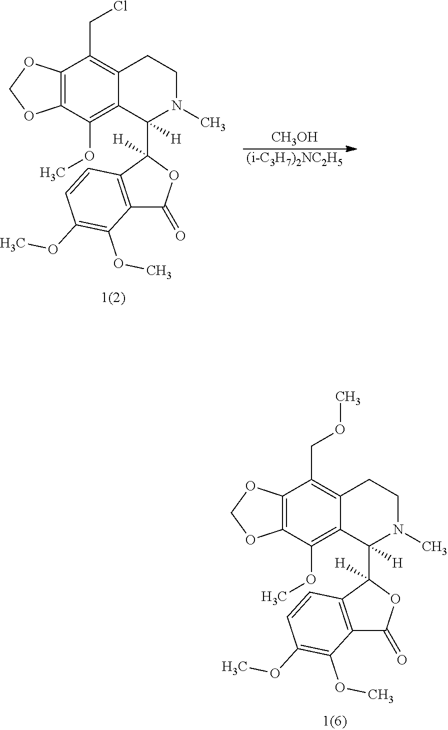

According to this invention is developed the method of obtaining 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-sulfonyl chloride 1 (7), that is consisted in action of chlorosulfonic acid on (R,S)-noscapine NSC according to the following diagram:

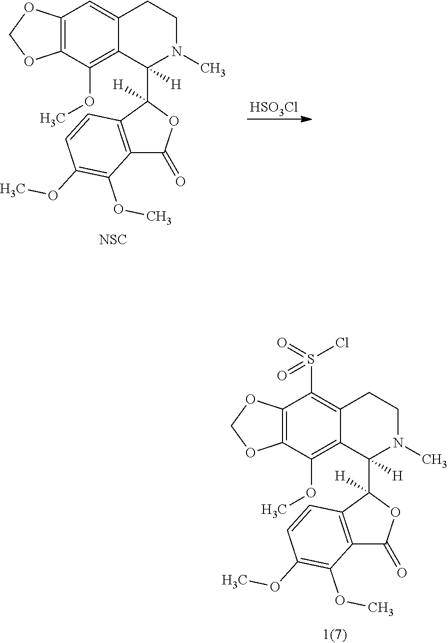

According to this invention is developed the method of obtaining the derivatives (R3S)-Noscapine of general formula 1.1, which is consisted in interaction 3-(9-Bromo-4-methoxy-6-methyl-5,6,7,8-tetrahydro 1,3 dioxolo-4,5-g isoquinoline-5-yl)-6,7-dimethoxy-3H-isobenzofuran-1-one or its iodide analog 1(1) in the presence of palladium catalyst aryl or heteroaryl by the boric derivatives of general formula 3 according to the following diagram:

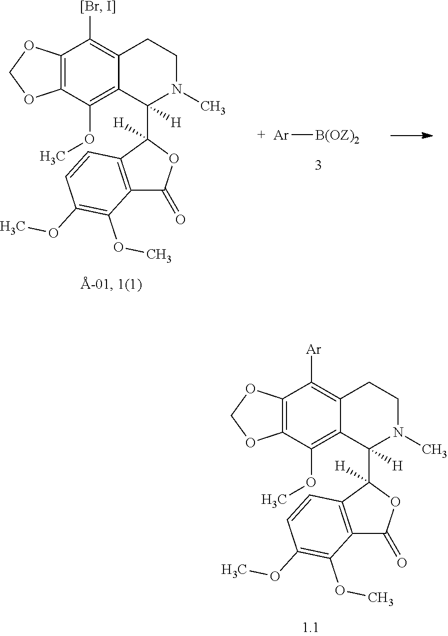

where Ar has values, indicated above with the determination of formula 1.1. As boron-containing arylating agents one can use arylboronic acids (Z═H), alkyl ethers of these acids 3 (Z═C₁₋₄ alkyl) or cyclic ethers of these acids, for example, 4,4,5,5-tetramethyl 1,3,2 dioxaboronic ether:

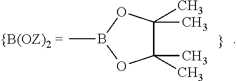

Crosslinking reactions are conducted in the polar aprotic solvent (dimethylformamide, N-methylpyrrolidone, dimethoxyethane or analogous), in the presence of 1-5 equivalents of inorganic base (carbonates, fluorides, bicarbonates or completely substituted phosphates of alkaline and alkaline earth metals, for example, cesium carbonate, fluoride of potassium, and also silver phosphate) and 5-25 molar % catalyst, as which use chloride or acetate of palladium, and also their complexes with the organophosphorus ligands, such as triphenylphosphine. The reaction is carried out with the heating at a temperature 100-170 C, under the conditions for microwave irradiation or without it. Most Preferred is the stereospecific method of the synthesis of the derivatives of Noscapine of general formula 1.1, that is characterized by the fact that the crosslinking combination A 01 and (get) arylboronic of acids are carried out in the polar aprotic solvents (for example, dimethoxyethane) in the presence of 3-4 equivalents of cesium carbonate even 10-20 mol. % complex of chloride palladium with triphenylphosphine with 130-150° C. under the action of microwave irradiation. According to this invention is developed the method of obtaining the derivatives (R3S)-Noscapine of general formula 1.2, which is consisted in interaction 3-(9-chloromethyl-4-methoxy-6-methyl-5,6,7,8-tetrahydro 1,3-di-oxolo 4,5-g isoquinoline-5-yl)-6,7-dimethoxy-3H-isobenzofuran-1-one 1 (2) with amines R³R⁴NH of general formula 4 according to the following diagram:

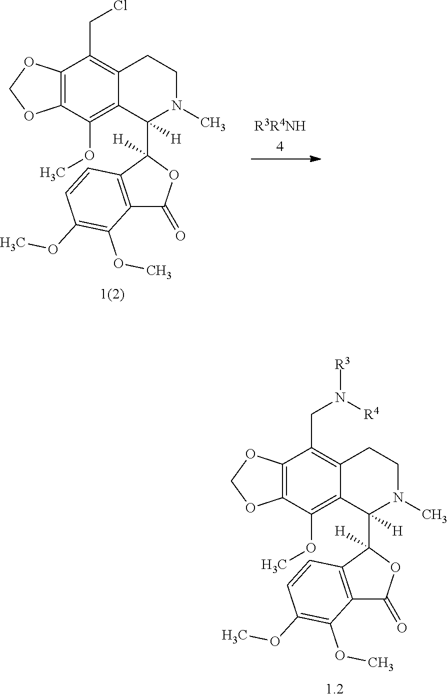

According to this invention the developed method of obtaining the derivatives (R,S)-noscapine of general formula 1.2 consists in the reductive amination of 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carbaldehyde 1 (3) by amines of general Formula 4 on organic solvent according to the following diagram:

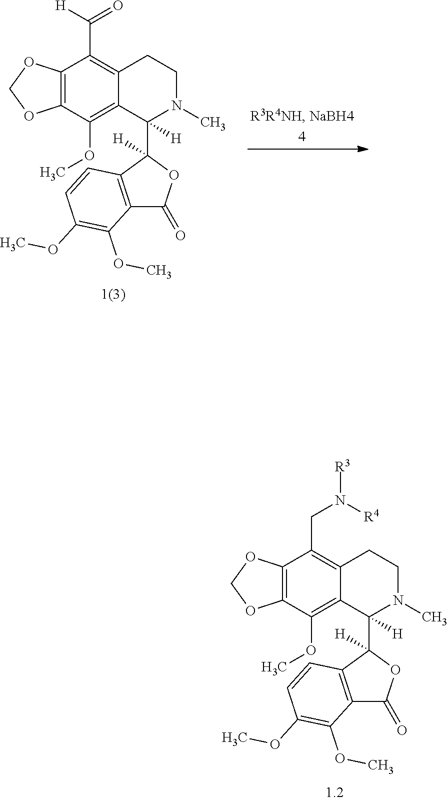

According to this invention the developed method of obtaining the derivatives (R,S)-noscapine of general formula 1.3 consists in interaction 5-(4,5-dimethoxy-3-oxo 1,3-dihydroisobenzofuran-1-yl)-4-methoxy-6-methyl-5,6,7,8-tetrahydro-1, 3 dioxolo 4,5-g isoquinoline-9-sulfonyl chloride 1 (7) with amines of general Formula 4 according to the following diagram:

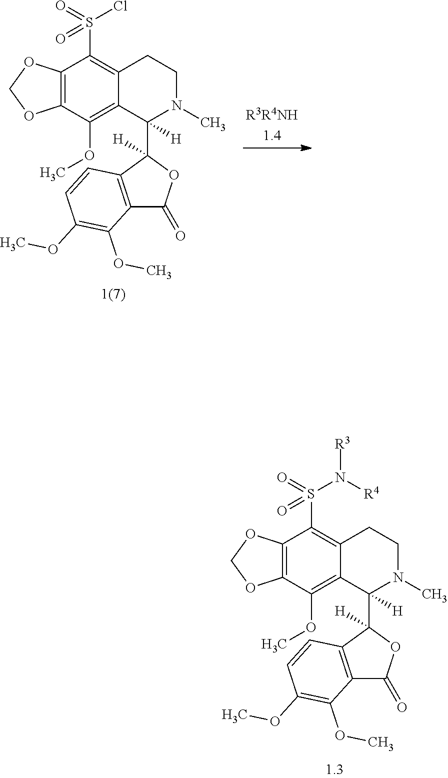

Furthermore, the compounds of the general Formula I present invention can form hydrates or pharmaceutical acceptable salts. For obtaining the salts can be used inorganic acids and organic acids, for example hydrochloric acid, hydrobromic acid, hydroiodic acid, sulfuric acid, phosphoric acid, formic acid, acetic acid, propionic acid, trifluoracetic acid, maleic acid, tartaric acid, methanesulfonic acid, benzenesulfonic acid, paratoluenesulfonic acid.

Also disclosed are combinatorial libraries for determining lead compounds, which include at least two or more compounds of general Formulas I, II, or III.

### III. Pharmaceutical Compositions

The noscapine analogs, their prodrugs and metabolites, and pharmaceutically acceptable salts, as described herein, can be incorporated into pharmaceutical compositions and used to treat or prevent a condition or disorder in a subject susceptible to such a condition or disorder, and/or to treat a subject suffering from the condition or disorder. Optically active compounds can be employed as racemic mixtures, as pure enantiomers, or as compounds of varying enantiomeric purity. The pharmaceutical compositions described herein include the noscapine analogs, their prodrugs and metabolites, and pharmaceutically acceptable salts, as described herein, and a pharmaceutically acceptable carrier and/or excipient.

The manner in which the compounds are administered can vary. The compositions are preferably administered orally (e.g., in liquid form within a solvent such as an aqueous or non-aqueous liquid, or within a solid carrier). Preferred compositions for oral administration include pills, tablets, capsules, caplets, syrups, and solutions, including hard gelatin capsules and time-release capsules. Compositions may be formulated in unit dose form, or in multiple or subunit doses. Preferred compositions are in liquid or semisolid form. Compositions including a liquid pharmaceutically inert carrier such as water or other pharmaceutically compatible liquids or semisolids may be used. The use of such liquids and semisolids is well known to those of skill in the art.

The compositions can also be administered via injection, i.e., intraveneously, intramuscularly, subcutaneously, intraperitoneally, intraarterially, intrathecally; and intracerebroventricularly. Intravenous administration is a preferred method of injection. Suitable carriers for injection are well known to those of skill in the art, and include 5% dextrose solutions, saline, and phosphate buffered saline. The compounds can also be administered as an infusion or injection (e.g., as a suspension or as an emulsion in a pharmaceutically acceptable liquid or mixture of liquids).

The formulations may also be administered using other means, for example, rectal administration. Formulations useful for rectal administration, such as suppositories, are well known to those of skill in the art. The compounds can also be administered by inhalation (e.g., in the form of an aerosol either nasally or using delivery articles of the type set forth in U.S. Pat. No. 4,922,901 to Brooks et al., the disclosure of which is incorporated herein in its entirety); topically (e.g., in lotion form); or transdermally (e.g., using a transdermal patch, using technology that is commercially available from Novartis and Alza Corporation). Although it is possible to administer the compounds in the form of a bulk active chemical, it is preferred to present each compound in the form of a pharmaceutical composition or formulation for efficient and effective administration.

Exemplary methods for administering such compounds will be apparent to the skilled artisan. The usefulness of these formulations may depend on the particular composition used and the particular subject receiving the treatment. These formulations may contain a liquid carrier that may be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

Preferably, the compositions are administered such that active ingredients interact with regions where microbial infections are located. The compounds described herein are very potent at treating these microbial infections.

In certain circumstances, the compounds described herein can be employed as part of a pharmaceutical composition with other compounds intended to prevent or treat a particular microbial infection, i.e., combination therapy. In addition to effective amounts of the compounds described herein, the pharmaceutical compositions can also include various other components as additives or adjuncts.

Combination Therapy

The combination therapy may be administered as (a) a single pharmaceutical composition which comprises a noscapine analog as described herein, or its prodrugs or metabolites, or pharmaceutically acceptable salts, at least one additional pharmaceutical agent described herein, and a pharmaceutically acceptable excipient, diluent, or carrier; or (b) two separate pharmaceutical compositions comprising (i) a first composition comprising a noscapine analog as described herein and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions can be administered simultaneously or sequentially and in any order.

In use in treating or preventing microbial disease, the noscapine analog(s) can be administered together with at least one other antimicrobial agent as part of a unitary pharmaceutical composition. Alternatively, it can be administered apart from the other antimicrobial agents. In this embodiment, the noscapine analog and the at least one other antimicrobial agent are administered substantially simultaneously, i.e. the compounds are administered at the same time or one after the other, so long as the compounds reach therapeutic levels for a period of time in the blood.

Combination therapy involves administering the noscapine analog, as described herein, or a pharmaceutically acceptable salt or prodrug of the noscapine analog, in combination with at least one anti-microbial agent, ideally one which functions by a different mechanism (i.e., by penetrating the bacterial, viral, or fungal cell wall, or interfering with one or more receptors and/or enzymes in the bacteria, virus, or fungus).

Representative Antiviral Agents

Some antiviral agents which can be used for combination therapy include agents that interfere with the ability of a virus to infiltrate a target cell. The virus must go through a sequence of steps to do this, beginning with binding to a specific “receptor” molecule on the surface of the host cell and ending with the virus “uncoating” inside the cell and releasing its contents. Viruses that have a lipid envelope must also fuse their envelope with the target cell, or with a vesicle that transports them into the cell, before they can uncoat.

There are two types of active agents which inhibit this stage of viral replication. One type includes agents which mimic the virus-associated protein (VAP) and bind to the cellular receptors, including VAP anti-idiotypic antibodies, natural ligands of the receptor and anti-receptor antibodies, receptor anti-idiotypic antibodies, extraneous receptor and synthetic receptor mimics. The other type includes agents which inhibit viral entry, for example, when the virus attaches to and enters the host cell. For example, a number of “entry-inhibiting” or “entry-blocking” drugs are being developed to fight HIV, which targets the immune system white blood cells known as “helper T cells”, and identifies these target cells through T-cell surface receptors designated “CD4” and “CCR5”. Thus, CD4 and CCR5 receptor inhibitors such as amantadine and rimantadine, can be used to inhibit viral infection, such as HIV, influenza, and hepatitis B and C viral infections. Another entry-blocker is pleconaril, which works against rhinoviruses, which cause the common cold, by blocking a pocket on the surface of the virus that controls the uncoating process.

Further antiviral agents that can be used in combination with the noscapine analogs described herein include agents which interfere with viral processes that synthesize virus components after a virus invades a cell. Representative agents include nucleotide and nucleoside analogues that look like the building blocks of RNA or DNA, but deactivate the enzymes that synthesize the RNA or DNA once the analogue is incorporated. Acyclovir is a nucleoside analogue, and is effective against herpes virus infections. Zidovudine (AZT), 3TC, FTC, and other nucleoside reverse transcriptase inhibitors (NRTI), as well as non-nucleoside reverse transcriptase inhibitors, can also be used. Integrase inhibitors can also be used.

Once a virus genome becomes operational in a host cell, it then generates messenger RNA (mRNA) molecules that direct the synthesis of viral proteins. Production of mRNA is initiated by proteins known as transcription factors, and certain active agents block attachment of transcription factors to viral DNA.

Other active agents include antisense oligonucleotides and ribozymes (enzymes which cut apart viral RNA or DNA at selected sites).

Some viruses, such as HIV, include protease enzymes, which cut viral protein chains apart so they can be assembled into their final configuration. Protease inhibitors are another type of antiviral agent that can be used in combination with the noscapine analogs described herein.

The final stage in the life cycle of a virus is the release of completed viruses from the host cell. Some active agents, such as zanamivir (Relenza) and oseltamivir (Tamiflu) treat influenza by preventing the release of viral particles by blocking a molecule named neuraminidase that is found on the surface of flu viruses.

Still other active agents function by stimulating the patient's immune system. Interferons, including pegylated interferons, are representative compounds of this class. Interferon alpha is used, for example, to treat hepatitis B and C. Various antibodies, including monoclonal antibodies, can also be used to target viruses.

Representative Antibacterial Compounds

Examples of antibacterial compounds include, but are not limited to, aminoglycosides, ansamycins, carbacephems, carbapenems, cephalosporins (First, Second, Third, Fourth and Fifth Generation), glycopeptides, macrolides, monobactams, penicillins and beta-lactam antibiotics, quinolones, sulfonamides, and tetracyclines.

Representative aminoglycosides include Amikacin, Gentamicin, Kanamycin, Neomycin, Netilmicin, Streptomycin, Tobramycin, and Paromomycin. Representative ansamycins include Geldanamycin and Herbimycin. These agents function by binding to the bacterial 30S or 50S ribosomal subunit, inhibiting the translocation of the peptidyl-tRNA from the A-site to the P-site and also causing misreading of mRNA, leaving the bacterium unable to synthesize proteins vital to its growth.

Loracarbef is a representative carbacephem. Representative carbapenems include Ertapenem, Doripenem, Imipenem/Cilastatin, and Meropenem.

Representative first generation cephalosporins include Cefadroxil, Cefazolin, Cefalotin, Cefalothin, and Cefalexin. Representative second generation cephalosporins include Cefaclor, Cefamandole, Cefoxitin, Cefprozil, and Cefuroxime. Representative third generation cephalosporins include Cefixime, Cefdinir, Cefditoren, Cefoperazone, Cefotaxime, Cefpodoxime, Ceftazidime, Ceftibuten, Ceftizoxime, and Ceftriaxone.

Cefepime is a representative fourth generation cephalosporin, and Ceftobiprole is a representative fifth generation cephalosporin.

Representative glycopeptides include Teicoplanin and Vancomycin, which function by inhibiting peptidoglycan synthesis.

Representative macrolides include Azithromycin, Clarithromycin, Dirithromycin, Erythromycin, Roxithromycin, Troleandomycin, Telithromycin, and Spectinomycin, which function by inhibiting bacterial protein biosynthesis by binding irreversibly to the subunit 50S of the bacterial ribosome, thereby inhibiting translocation of peptidyl tRNA.

Aztreonam is a representative monobactam.

Representative penicillins include Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, and Ticarcillin. These can be administered with an agent which inhibits beta-lactamase enzymatic activity, such as potassium clavanulate or clavulanic acid.

Representative quinolones include Ciprofloxacin, Enoxacin, Gatifloxacin, Levofloxacin, Lomefloxacin, Moxifloxacin, Norfloxacin, Ofloxacin, and Trovafloxacin.

Representative sulfonamides include Mafenide, Prontosil, Sulfacetamide, Sulfamethizole, Sulfanilimide, Sulfasalazine, Sulfisoxazole, Trimethoprim, and Trimethoprim-Sulfamethoxazole (Co-trimoxazole) (TMP-SMX).

Representative tetracyclines include Demeclocycline, Doxycycline, Minocycline, Oxytetracycline, and Tetracycline.

Other antibacterial agents include, for example, Arsphenamine, Chloramphenicol, Clindamycin, Lincomycin, Ethambutol, Fosfomycin, Fusidic acid, Furazolidone, Isoniazid, Linezolid, Metronidazole, Mupirocin, Nitrofurantoin, Platensimycin, Pyrazinamide, Quinupristin/Dalfopristin, Rifampin or Rifampicin, and Timidazole.

Representative Antifungal Compounds

Examples of known antifungal agents which can be used for combination therapy include, but are not limited to AMB (Amphotericin B deoxycholate), also known as Fungizone, ABLC (Amphotericin B lipid complex), also known as Abelcet, ABCD (Amphotericin B colloidal dispersion), also known as Amphotec, LAMB (Liposomal amphotericin B), also known as AmBisome, Echinocandin, also known as Aspofungin, Micafungin or Anidulafungin.

Other examples of antifungal agents include, but are not limited to, Posaconazole, Ketoconazole, Fluconazole PO, Clotrimazole troche, Nystatin oral suspension, Voriconazole, Griseofulvin, Terbinafine, and Flucytosine.

Any of the above-mentioned compounds can be used in combination therapy with the noscapine analogs.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder.

When treating microbial infections, an effective amount of the noscapine analogue is an amount sufficient to suppress the growth and proliferation of the microbe(s). Microbial infections can be prevented, either initially, or from re-occurring, by administering the compounds described herein in a prophylactic manner. Preferably, the effective amount is sufficient to obtain the desired result, but insufficient to cause appreciable side effects.

The effective dose can vary, depending upon factors such as the condition of the patient, the severity of the microbial infection, and the manner in which the pharmaceutical composition is administered. The effective dose of compounds will of course differ from patient to patient, but in general includes amounts starting where desired therapeutic effects occur but below the amount where significant side effects are observed.

The compounds, when employed in effective amounts in accordance with the method described herein, are effective at inhibiting the proliferation of certain microbes, but do not significantly effect normal cells.

For human patients, the effective dose of typical compounds generally requires administering the compound in an amount of at least about 1, often at least about 10, and frequently at least about 25 μg/24 hr/patient. The effective dose generally does not exceed about 500, often does not exceed about 400, and frequently does not exceed about 300 μg/24 hr/patient. In addition, administration of the effective dose is such that the concentration of the compound within the plasma of the patient normally does not exceed 500 ng/mL and frequently does not exceed 100 ng/mL.

IV. Methods of Using the Compounds and/or Pharmaceutical Compositions

The compounds can be used to treat or prevent microbial infections, including infections by viruses, bacteria, and/or fungi, and/or to inhibit microbial replication. Many microbes use the cytoskeletal machinery of the cell to assist in movement and replication. The compounds, compositions, and methods inhibit the movement of the microbes, which use the microtubules of the cell for transport.

Representative Viruses Whose Replication can be Inhibited

The microorganisms include viruses such as the ebola virus (Yonezawa, A., Cavrois, M., and Greene, W. C. (2005) Studies of ebola virus glycoprotein-mediated entry and fusion by using pseudotyped human immunodeficiency virus type 1 virions: involvement of cytoskeletal proteins and enhancement by tumor necrosis factor alpha. J. Virol. 79, 918-926), the polyoma virus (Sanjuan, N., Porras, A., and Otero, J. (2003). Microtubule-dependent intracellular transport of murine polyomavirus. Virology 313, 105-116.), the influenza virus (Lakadamyali, M., Rust, M. J., Babcock, H. P., and Zhuang, X. (2003). Visualizing infection of individual influenza viruses. Proc. Natl. Acad. Sci. USA 100, 9280-9285.), simian virus 40 (Marsh, M., and Helenius, A. (2006). Virus entry: Open sesame. Cell 124, 741-754, Feb. 24, 2006), HIV (McDonald, D., Vodicka, M. A., Lucero, G., Svitkina, T. M., Borisy, G. G., Emerman, M., and Hope, T. J. (2002). Visualization of the intracellular behavior of HIV in living cells. J. Cell Biol. 159, 441-452.), herpes viruses (Greber, U. F. (2005). Viral trafficking violations in axons—the herpes virus case. Proc. Natl. Acad. Sci. USA 102, 5639-5640.), retroviruses such as the Human foamy virus (HFV) (Petit, C., Giron, M. L., Tobaly-Tapiero, J., Bittoun, P., Real, E., Jacob, Y., Tordo, N., De The, H., and Saib, A. (2003). Targeting of incoming retroviral Gag to the centrosome involves a direct interaction with the dynein light chain 8. J. Cell Sci. 116, 3433-3442.), and the Mason-Pfizer monkeyvirus (M-PMV) (Sfakianos, J. N., LaCasse, R. A., and Hunter, E. (2003). The M-PMV cytoplasmic targeting-retention signal directs nascent Gag polypeptides to a pericentriolar region of the cell. Traffic 4, 660-670.), as well as other viruses, including those from the viral families Adenoviridae, Papillomaviridae, Parvoviridae, Herpesviridae, Poxyiridae, Hepadnaviridae, Polyomaviridae, and Circoviridae, which all use the microtubules of the cell for transport and replication.

Representative Bacteria Whose Replication can be Inhibited

Additionally, several bacterial species have been shown to use host cell cytoskeletal machinery for invasion into the host cell, such as the Shigella and Salmonella species (Gruenheid S, Finlay B B, Microbial Pathogenesis and Cytoskeletal Function, Nature. 2003 Apr. 17; 422(6933):775-81), Actinobacillus speices (Meyer, Rose, Lipmann, and Taylor, Microtubules Are Associated with Intracellular Movement and Spread of the Periodontopathogen Actinobacillus actinomycetemcomitans, Infection and Immunity, December 1999, p. 6518-6525), Francisella tularensis spp. (Craven R R, Hall J D, Fuller J R, Taft-Benz S, Kawula T H, Francisella tularensis invasion of lung epithelial cells. Infect Immun. 2008 July; 76(7):2833-42. Epub 2008 Apr. 21), and Campylobacter jejuni as well as Citrobacter freundii spp. (T A Oelschlaeger, P Guerry, and D J Kopecko, Unusual microtubule-dependent endocytosis mechanisms triggered by Campylobacter jejuni and Citrobacter freundii., Proc Natl Acad Sci USA. 1993 Jul. 15; 90(14): 6884-6888). Shigella flexneri, E. coli, Yersinia enterocolitica, and Listeria monocytogenes have also been shown to use the cytocellular machinery of epithelial cells for invasion into a host cell. (Invasive Properties of E. coli Strains Associated With Crohn's Disease. Curr Opin Gastroenterol. 2007; 23(1):16-20.)

Representative Fungi Whose Replication can be Inhibited

Fungi also hijack the cytoskeletal machinery of the cell to invade host cells. (Steinberg G., (2007) On the move: endosomes in fungal growth and pathogenicity. Nat Rev Microbiol. 2007 April; 5(4):309-16. Epub 2007 Feb. 26) Several fungal species known to use the cytoskeletal machinery for invasion are Candida albicans, Paracoccidioides brasiliensis, Saccharomyces cerevisiae, and Schizosaccharomyces pombe. (Filler, Sheppard, Fungal Invasion of Normally Non-Phagocytic Host Cells, PLoS Pathog 2(12): e129. doi:10.1371/journal.ppat.0020129; Fischer R, Zekert N, Takeshita N., Polarized growth in fungi—interplay between the cytoskeleton, positional markers and membrane domains., Mol. Microbiol. 2008 May; 68(4):813-26. Epub 2008 Apr. 8.)

The compounds can also be used as adjunct therapy in combination with existing therapies in the management of the aforementioned types of infections. In such situations, it is preferably to administer the active ingredients to a patient in a manner that optimizes effects upon microbes, including drug resistant microbes, while minimizing effects upon normal cell types. While this is primarily accomplished by virtue of the behavior of the compounds themselves, this can also be accomplished by targeted drug delivery and/or by adjusting the dosage such that a desired effect is obtained without meeting the threshold dosage required to achieve significant side effects.

The following examples are provided to illustrate the present invention, and should not be construed as limiting thereof. In these examples, all parts and percentages are by weight, unless otherwise noted. Reaction yields are reported in mole percentages.

### Example 1

### Synthesis of 9-Aminonoscapine

Experimental

General:

See earlier comment. ¹H NMR and ¹³C NMR spectra were measured in CDCl₃ on INOVA 400 NMR spectrometer. All proton NMR spectra were recorded at 400 MHz and were referenced with residual chloroform (7.27 ppm). All carbon NMR spectra were recorded at 100 MHz and were referenced with 77.27 ppm resonance of residual chloroform. Abbreviations for signal coupling are as follows: s, singlet; d, doublet; t, triplet; q, quartet; m, multiplet. Infrared spectra were recorded on sodium chloride discs on Mattson Genesis II FT-IR. High resolution mass spectra were collected on Thermo Finnigan LTQ-FT Hybrid mass spectrophotometer using 3-nitrobenzyl alcohol, in some cases with addition of LiI as a matrix. Melting points were determined using a Thomas Hoover melting point apparatus and were uncorrected. All reactions were conducted in oven-dried (125° C.) glassware under an atmosphere of dry argon. All common reagents and solvents were obtained from commercial suppliers and used without further purification unless otherwise indicated. Solvents were dried by standard methods. The reactions were monitored by thin layer chromatography (TLC) using silica gel 60 F254 (Merck) precoated aluminum sheets. Flash chromatography was carried out on standard grade silica gel (230-400 mesh).

Synthesis of 9-aminonoscapine was shown in Scheme 1. Briefly, noscapine (1) was dissolved minimum amount of 48% hydrobromic acid and then cautiously added freshly prepared bromine water. The reaction mixture stirred for 1 h at 25° C. and the resultant mixture was basified to pH 10 to afford 9-bromonoscapine in 82% yield. Refluxing compound 2 in DMF with sodium azide and sodium iodide for 15 hours gave its azido derivative (3) in quantitative yield. Reduction of azido derivative with tin chloride in the presence of thiophenol and triethylamine in THF for 2 h at 25° C. afforded the title compound, 9-aminonoscapine (4) in 83% yield.

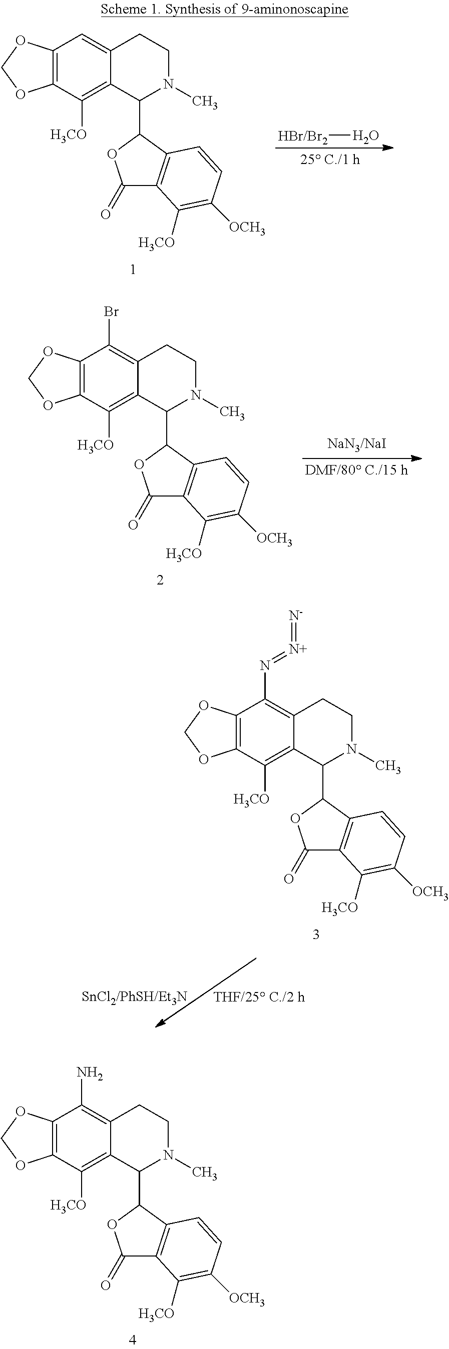

### (S)-3-((R)-9-bromo-4-methoxy-6-methyl-5,6,7,8-tetrahydro-[1,3]dioxolo[4,5-g]isoquinolin-5-yl)-6,7-dimethoxyisobenzofuran-1(3H)-one (2)

To a flask containing noscapine (20 g, 48.4 mmol) was added minimum amount of 48% hydrobromic acid solution (˜40 ml) to dissolve or make a suspension of the reactant. To the reaction mixture was added freshly prepared bromine water (˜250 ml) drop wise until an orange precipitate appeared. The reaction mixture was then stirred at room temperature for 1 h to attain completion, neutralized to pH 10 using ammonia solution to afford solid precipitate. The solid precipitate was recrystallized with ethanol to afford bromo-substituted noscapine. Yield: 82%; mp 169-170° C.; IR: 2945 (m), 2800 (m), 1759 (s), 1612 (m), 1500 (s), 1443 (s), 1263 (s), 1091 (s), 933 (w) cm⁻¹; ¹H NMR (CDCl₃, 400 MHz), δ 7.04 (d, 1H, J=7 Hz), 6.32 (d, 1H, J=7 Hz), 6.03 (s, 2H), 5.51 (d, 1H, J=4 Hz), 4.55 (d, 1H, J=4 Hz), 4.10 (s, 3H), 3.98 (s, 3H), 3.89 (s, 3H), 2.52 (s, 3H), 2.8-1.93 (m, 4H); ¹³C NMR (CDCl₃, 100 MHz), δ 167.5, 151.2, 150.5, 150.1, 148.3, 140.0, 135.8, 130.8, 120.3, 120.4, 120.1, 105.3, 100.9, 100.1, 87.8, 64.4, 56.1, 56.0, 55.8, 51.7, 41.2, 27.8; MS (FAB): m/z (relative abundance, %), 494 (93.8), 492 (100), 300 (30.5), 298 (35.4); MALDI: m/z 491.37 (M⁺), 493.34; ESI/tandem mass spectrometry: parent ion masses, 494, 492; daughter ion masses (intensity, %), 433 (51), 431 (37), 300 (100), 298 (93.3); HRMS (ESI): m/z Calcd. for C₂₂H₂₃BrNO₇ (M+1), 493.3211; Found, 493.3215 (M+1).

### (S)-3-((R)-9-azido-4-methoxy-6-methyl-5,6,7,8-tetrahydro-[1,3]dioxolo[4,5-g]isoquinolin-5-yl)-6,7-dimethoxyisobenzofuran-1(3H)-one (3)

To a solution of compound 2 (2.0 g, 4.063 mmol) in DMF (20 mL) were added sodium azide (2.641 g, 40.63 mmol) and sodium iodide (0.609 g, 4.063 mmol) and the reaction mixture was stirred at 80° C. for 15 h to attain completion. Then the solvent was removed in vacuo and the resultant residue was dissolved in chlorofrom (40 mL), washed with water (2×40 mL), dried over sodium sulfate and concentrated to obtain the titled compound 3, which was recrystallized with ethanol in hexane (10:90) to afford brown crystals. Yield, 89%; mp 177.2-178.1° C.; IR: 1529, 1362 cm⁻¹; ¹H NMR (CDCl₃, 400 MHz): δ 7.05 (d, 1H, J=7.0 Hz), 6.4 (d, 1H, J=7.0 Hz), 6.01 (s, 2H), 5.85 (d, 1H, J=4.4 Hz), 4.40 (d, 1H, J=4.4 Hz), 4.15 (s, 3H), 3.88 (s, 3H), 3.84 (s, 3H), 2.75-2.62 (m, 2H), 2.60-2.56 (m, 2H), 2.51 (s, 3H); ¹³C NMR (CDCl₃, 100 MHz): δ 169.2, 157.7, 152.6, 147.9, 142.2, 140.5, 135.0, 134.0, 123.5, 121.8, 119.7, 119.3, 114.1, 100.5, 87.4, 64.1, 56.7, 56.5, 56.2, 51.4, 39.2, 27.2; HRMS (ESI): m/z Calcd. for C₂₂H₂₃N₄O₇ (M+1), 455.4335; Found, 455.4452 (M+1).

### (S)-3-OR)-9-amino-4-methoxy-6-methyl-5,6,7,8-tetrahydro-[1,3]dioxolo[4,5-asoqluinolin-5-yl)-6,7-dimethoxyisobenzofuran-1(3H)-one (4)

To a 50-mL of round-bottomed flask containing a solution of SnCl₂ in THF (10 mL) were added thiophenol and triphenylamine. The reaction mixture was added slowly to a solution of azido-noscapine (3, 0.2 g, 0.440 mmol) in THF (5 mL) and the reaction mixture stirred at room temperature. The reaction progress was monitored by thin-layer chromatography at 30 minutes intervals. The reaction was found to be completed after 2 h, the solvent was removed in vacuo. The residue was diluted with chloroform (20 ml) and was added sodium hydroxide solution (20 mL). the aqueous phase was separated and extracted with chloroform (2×20 mL). the combined organic phase was dried over sodium sulfate and concentrated to obtain amino-noscapine as colorless oil, which was then treated with ethereal HCl to obtain its salt as white crystals. Yield, 83%; mp (HCl. Salt) 112.2-112.6° C.; IR: 1725, 1362 cm⁻¹; ¹H NMR (CDCl₃, 400 MHz): δ 7.12 (d, 1H, J=7.4.0 Hz), 7.02 (d, 1H, J=7.4 Hz), 6.02 (s, 2H), 5.92 (d, 1H, J=4.0 Hz), 4.42 (d, 1H, J=4.0 Hz), 4.20 (bs, 2H), 4.02 (s, 3H), 3.85 (s, 3H), 3.80 (s, 3H), 2.74-2.64 (m, 2H), 2.61-2.56 (m, 2H), 2.52 (s, 3H); ¹H NMR (CDCl3+D₂O, 400 MHz): δ 7.12 (d, 1H, J=7.4.0 Hz), 7.02 (d, 1H, J=7.4 Hz), 6.02 (s, 2H), 5.92 (d, 1H, J=4.0 Hz), 4.42 (d, 1H, J=4.0 Hz), 5.12 (bs, confirms NH₂ group), 4.02 (s, 3H), 3.85 (s, 3H), 3.80 (s, 3H), 2.74-2.64 (m, 2H), 2.61-2.56 (m, 2H), 2.52 (s, 3H); ¹³C NMR (CDCl₃, 100 MHz): δ 169.5, 156.8, 152.6, 147.8, 142.7, 141.8, 135.0, 134.2, 123.2, 120.8, 119.9, 119.4, 114.1, 100.8, 87.6, 63.7, 56.8, 56.4, 56.1, 51.4, 39.2, 27.5; HRMS (ESI): m/z Calcd. for C₂₂H₂₄N₂O₇ (M+1), 428.3481; Found, 428.1562 (M+1).

### HPLC Purity and Peak Attributions:

The HPLC purity was determined following two different methods using varied solvent systems.
Method 1: Ultimate Plus, LC Packings, Dionex, C₁₈ column (pep Map 100, 3 μm, 100 Å particle size, ID: 1000 μm, length: 15 cm) with solvent systems A (0.1% formic acid in water) and B (acetonitrile), gradient, 25 min run at a flow of 40 μL/min. Retention time for 9-amino-nos is 18.30 min. HPLC purity was 95%.
Method 2: Ultimate Plus, LC Packings, Dionex, C₁₈ column (pep Map 100, 3 μm, 100 Å particle size, ID: 1000 μm, length: 15 cm) with solvent systems A (0.1% formic acid in water) and B (methanol), gradient, 25 min run at a flow of 40 μL/min.

Retention time for 9-amino-nos is 18.96 min. HPLC purity was 94%.

### Example 2

### Synthesis of 9-Chloro-Noscapine

Chemistry: ¹H NMR and ¹³C NMR spectra were measured by 400 NMR spectrometer in a CDCl₃ solution and analyzed by INOVA. Proton NMR spectra were recorded at 400 MHz and were referenced with residual chloroform (7.27 ppm). Carbon NMR spectra were recorded at 100 MHz and were referenced with 77.27 ppm resonance of residual chloroform. Abbreviations for signal coupling are as follows: s, singlet; d, doublet; t, triplet; q, quartet; m, multiplet. Infrared spectra were recorded on sodium chloride discs on Mattson Genesis II FT-IR. High resolution mass spectra were collected on Thermo Finnigan LTQ-FT Hybrid mass spectrophotometer using 3-nitrobenzyl alcohol or with addition of L11 as a matrix. Melting points were determined using a Thomas-Hoover melting point apparatus and were uncorrected. All reactions were conducted with oven-dried (125° C.) reaction vessels in dry argon. All common reagents and solvents were obtained from Aldrich and were dried using 4 Å molecular sieves. The reactions were monitored by thin layer chromatography (TLC) using silica gel 60 F254 (Merck) on precoated aluminum sheets. Flash chromatography was carried out on standard grade silica gel (230-400 mesh).

### (S)-3-(R)-9-chloro-4-methoxy-6-methyl-5,6,7,8-tetrahydro-[1,3]dioxolo[4,5-g]iso-quinolin-5-yl)-6,7-dimethoxyisobenzofuran-1(3H)-one

To a stirred solution of noscapine (5 g, 12.01 mmol) in chloroform (200 ml), a solution of sulfuryl chloride (4.897 g, 36.28 mmol) in 100 ml chloroform was added drop wise over a period of 1 hour at 5-10° C. The reaction mixture was allowed to attain room temperature and stirring was continued for 10 hours. The reaction progress was monitored using thin layer chromatography (7% methanol in chloroform). The reaction mixture was poured into 300 ml of water and extracted with chloroform (2×200 ml). The organic layer was washed with brine, dried over anhydrous magnesium sulfate and the solvent evaporated in vacuo to afford the crude product. Purification of the crude product using flash chromatography (silica gel, 230-400 mesh) with 7% methanol in chloroform as an eluent afforded the desired product, (S)-3-(R)-9-chloro-4-methoxy-6-methyl-5,6,7,8-tetrahydro-[1,3]dioxolo[4,5-g]isoquinolin-5-yl)-6,7-dimethoxyisobenzofuran-1(3H)-one (4). Yield: 90% (4.49 g), colorless needles; mp 169.0-169.1° C.; ¹H NMR (CDCl₃, 400 MHz): δ 7.14 (d, 1H, J=8.26 Hz), 6.41 (d, 1H, J=8.26 Hz), 5.93 (s, 2H), 5.27 (d, 1H, J=4.31 Hz), 4.20 (d, 1H, J=4.32 Hz), 3.99 (s, 3H), 3.87 (s, 3H), 3.83 (s, 3H), 2.79-2.65 (m, 2H), 2.54-2.46 (m, 2H), 2.35 (s, 3H); ¹³C NMR (CDCl₃, 100 MHz): δ 167.7, 152.4, 147.5, 139.3, 134.9, 126.1, 120.3, 118.4, 108.5, 102.3, 93.5, 81.9, 64.2, 61.8, 59.6, 57.7, 54.9, 46.1, 45.2, 39.8, 20.6, 18.6; HRMS (ESI): m/z Calcd. for C₂₂H₂₃ClNO₇ (M+1), 448.11481; Found, 448.11482 (M+1).

### Example 3

### Preparation of 9-Nitro-Nos((S)-6,7-dimethoxy-3-((R)-4-methoxy-6-methyl-9-nitro-5,6,7,8-tetrahydro-[1,3]dioxolo[4,5-g]isoquinolin-5-yl)isobenzofuran-1(3H)-one)

Chemistry:

¹H NMR and ¹³C NMR spectra were measured by 400 NMR spectrometer in a CDCl₃ solution and analyzed by INOVA. Proton NMR spectra were recorded at 400 MHz and were referenced with residual chloroform (7.27 ppm). Carbon NMR spectra were recorded at 100 MHz and were referenced with 77.27 ppm resonance of residual chloroform. Abbreviations for signal coupling are as follows: s, singlet; d, doublet; t, triplet; q, quartet; m, multiplet. Infrared spectra were recorded on sodium chloride discs on Mattson Genesis II FT-IR. High resolution mass spectra were collected on Thermo Finnigan LTQ-FT Hybrid mass spectrophotometer using 3-nitrobenzyl alcohol or with addition of LiI as a matrix. Melting points were determined using a Thomas-Hoover melting point apparatus and were uncorrected. All reactions were conducted with oven-dried (125° C.) reaction vessels in dry argon. All common reagents and solvents were obtained from Aldrich and were dried using 4 Å molecular sieves. The reactions were monitored by thin layer chromatography (TLC) using silica gel 60 F254 (Merck) on precoated aluminum sheets. Flash chromatography was carried out on standard grade silica gel (230-400 mesh).

To a solution of noscapine (4.134 g, 10 mmol) in acetonitrile (50 ml), silver nitrate (1.70 g, 10 mmol) and trifluoroacetic anhydride (5 ml, 35 mmol) were added. After one hour of reaction time, the reaction progress was monitored using thin layer chromatography (10% methanol in chloroform) and the reaction mixture was poured into 50 ml of water and extracted with chloroform (3×50 ml). The organic layer was washed with brine, dried over anhydrous MgSO₄ and the solvent was evaporated in vacuo. The desired product, (S)-6,7-dimethoxy-3-((R)-4-methoxy-6-methyl-9-nitro-5,6,7,8-tetrahydro-[1,3]dioxolo[4,5-g]isoquinolin-5-yl)isobenzofuran-1(3H)-one (9-nitro-nos) was obtained as yellow crystalline powder by flash chromatography (silica gel, 230-400 mesh) with 10% methanol in chloroform as an eluent. mp 178.2-178.4° C.; IR: 1529, 1362 cm-1; ¹H NMR (CDCl₃, 400 MHz): δ 7.27 (d, 1H, J=8.0 Hz), 7.08 (d, 1H, J=8.0 Hz), 6.02 (s, 2H), 5.91 (d, 1H, J=4.1 Hz), 4.42 (d, 1H, J=4.1 Hz), 4.09 (s, 3H), 3.89 (s, 3H), 3.83 (s, 3H), 2.74-2.64 (m, 2H), 2.61-2.56 (m, 2H), 2.52 (s, 3H); ¹³C NMR (CDCl₃, 100 MHz): δ 169.7, 157.2, 151.6, 147.5, 142.3, 140.5, 135.0, 134.2, 123.2, 120.8, 119.9, 119.4, 114.1, 100.8, 87.6, 63.7, 56.8, 56.4, 56.1, 51.4, 39.2, 27.0; HRMS (ESI): m/z Calcd. for C₂₂H₂₃N₂O₉ (M+1), 459.4821; Found, 459.4755 (M+1).

HPLC Purity and Peak Attributions:

The HPLC purity was determined following two different methods using varied solvent systems.

Method 1: Ultimate Plus, LC Packings, Dionex, C18 column (pep Map 100, 3 μm, 100 Å particle size, ID: 1000 μm, length: 15 cm) with solvent systems A (0.1% formic acid in water) and B (acetonitrile), gradient, 25 min run at a flow of 40 μL/min. Retention time for 9-nitro-nos is 19.30 min. HPLC purity was 96%. Method 2: Ultimate Plus, LC Packings, Dionex, C18 column (pep Map 100, 3 μm, 100 Å particle size, ID: 1000 μm, length: 15 cm) with solvent systems A (0.1% formic acid in water) and B (methanol), gradient, 25 min run at a flow of 40 μL/min. Retention time for 9-nitro-nos is 19.86 min. HPLC purity was 97%.

Discussion of Other Synthetic Approaches

The nitration reaction is a well-studied electrophilic substitution reaction in organic chemistry. Although, fuming nitric acid or 50% nitric acid in glacial acetic acid are extensively used for obtaining the nitrated product, the harsh oxidizing conditions of these reagents did not allow us to use these reagents for the nitration of noscapine. The lead compound, noscapine comprises of isoquinoline and benzofuranone ring systems joined by a labile C—C chiral bond and both these ring systems contain several vulnerable methoxy groups. Thus, achieving selective nitration at C-9 position without disruption and cleavage of these labile groups and C—C bonds was challenging. Treatment of noscapine with other nitrating agents like acetyl nitrate or benzoyl nitrate also resulted in epimerization or diastereoisomers (Lee, 2002). Next, inorganic nitrate salts like ammonium nitrate or silver nitrate were used in the presence of acidic media to achieve aromatic nitration (Crivello, 1981). After carefully titrating several conditions and reagents, the nitration of noscapine using trifluoroacetic anhydride (TFAA) was successfully accomplished. TFAA represents another commonly employed reagent and its extensive use is associated with its ability to generate a mixed anhydride, trifluoroacetyl nitrate that is a reactive nitrating agent (Crivello, 1981). Other reagents such as ammonium nitrate, sodium nitrate or silver nitrate in chloroform were also tried, but those provided low quantitative yields and had longer reaction times. Increased reaction rate and yields were obtained using a lower dielectric constant solvent, acetonitrile. The reaction was slightly exothermic and completed in one hour. The product remained in solution while the inorganic salt of trifluoroacetic acid precipitated and was removed by filtration.

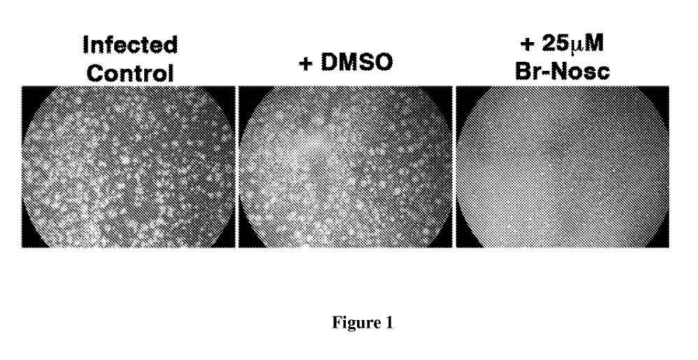

Thus, (S)-6,7-dimethoxy-3-((R)-4-methoxy-6-methyl-9-nitro-5,6,7,8-tetrahydro-[1,3]dioxolo[4,5-g]iso-quinolin-5-yl)isobenzofuran-1(3H)-one (9-nitro-nos) was prepared by the aromatic nitration of (S)-6,7-dimethoxy-3-((R)-4-methoxy-6-methyl-5,6,7,8-tetrahydro-[1,3]dioxolo[4,5-g] isoquinolin-5-yl)isobenzo-furan-1(3H)-one (noscapine) using silver nitrate in acetonitrile and TFAA at 25° C. (FIG. 1A). This method resulted in controlling the chemoselectivity of the reaction, in that aromatic substitution occurred at C-9 position of ring A of the isoquinoline nucleus. Absence of C-9 aromatic proton at δ 6.52-ppm in the ¹H NMR spectrum of the product confirmed the nitration at C-9 position. Furthermore, ¹³C NMR and HRMS data confirmed the structure of the compound.

### Example 4

### Synthesis of Halogenated Noscapine Analogues

### (S)-3-((R)-9-bromo-4-methoxy-6-methyl-5,6,7,8-tetrahydro-[1,3]dioxolo[4,5-g]isoquino-lin-5-yl)-6,7-dimethoxyisobenzofuran-1(3H)-one

To a flask containing noscapine (20 g, 48.4 mmol) was added minimum amount of 48% hydrobromic acid solution (˜40 ml) to dissolve or make a suspension of the reactant. To the reaction mixture was added freshly prepared bromine water (˜250 ml) drop wise until an orange precipitate appeared. The reaction mixture was then stirred at room temperature for 1 h to attain completion, adjusted to pH 10 using ammonia solution to afford solid precipitate. The solid precipitate was recrystallized with ethanol to afford bromo-substituted noscapine. Yield: 82%; mp 169-170° C.; IR: 2945 (m), 2800 (m), 1759 (s), 1612 (m), 1500 (s), 1443 (s), 1263 (s), 1091 (s), 933 (w) cm⁻¹; ¹H NMR (CDCl₃, 400 MHz), δ 7.04 (d, 1H, J=7 Hz), 6.32 (d, 1H, J=7 Hz), 6.03 (s, 2H), 5.51 (d, 1H, J=4 Hz), 4.55 (d, 1H, J=4 Hz), 4.10 (s, 3H), 3.98 (s, 3H), 3.89 (s, 3H), 2.52 (s, 3H), 2.8-1.93 (m, 4H); ¹³C NMR (CDCl₃, 100 MHz), δ 167.5, 151.2, 150.5, 150.1, 148.3, 140.0, 135.8, 130.8, 120.3, 120.4, 120.1, 105.3, 100.9, 100.1, 87.8, 64.4, 56.1, 56.0, 55.8, 51.7, 41.2, 27.8; MS (FAB): m/z (relative abundance, %), 494 (93.8), 492 (100), 300 (30.5), 298 (35.4); MALDI: m/z 491.37 (M+), 493.34; ESI/tandem mass spectrometry: parent ion masses, 494, 492; daughter ion masses (intensity, %), 433 (51), 431 (37), 300 (100), 298 (93.3); HRMS (ESI): m/z Calcd. for C₂₂H₂₃BrNO₇ (M+1), 493.3211; Found, 493.3215 (M+1).

### (S)-3-(R)-9-fluoro-4-methoxy-6-methyl-5,6,7,8-tetrahydro-[1,3]dioxolo[4,5-g]isoquino-lin-5-yl)-6,7-dimethoxyisobenzofuran-1(3H)-one

To a solution of bromonoscapine (1 g, 2.42 mmol) in anhydrous THF (20 ml) was added an excess of Amberlyst-A 26 (fluorine, polymer-supported, 2.5 g, 10 mequiv. of dry resin, the average capacity of the resin is 4 mequiv. per gram) and the reaction mixture refluxed for 12 hours. The resin was filtered off and the solvent removed to afford the crude product which was purified by flash column chromatography (ethyl acetate/hexane=4:1) to afford (S)-3-((R)-9-fluoro-4-methoxy-6-methyl-5,6,7,8-tetrahydro-[1,3]dioxolo[4,5-g]isoquinolin-5-yl)-6,7-dimethoxy-isobenzo-furan-1(3H)-one (3) as a light brown crystals. The recovery of resin was achieved by washing with 1 M NaOH and then rinsing thoroughly with water until neutrality to afford hydroxy-form of resin. It was then stirred overnight with 1 M aqueous hydrofluoric acid (250 ml), washed with acetone, ether and dried in a vacuum oven at 50° C. for 12 hours to afford the regenerated Amberlyst-A 26 (fluorine, polymer-supported). Yield: 74%, light brown crystals; mp 170.8-171.1° C.; ¹H NMR (CDCl₃, 400 MHz): δ 7.11 (d, 1H, J=8.0 Hz), 6.99 (d, 1H, J=8.0 Hz), 5.44 (s, 2H), 5.21 (d, 1H, J=4.1 Hz), 4.02 (d, 1H, J=4.1 Hz), 3.95 (s, 3H), 3.78 (s, 3H), 3.64 (s, 3H), 2.65-2.62 (m, 2H), 2.51-2.47 (m, 2H), 2.30 (s, 3H); ¹³C NMR (CDCl₃, 100 MHz): δ 167.5, 152.9, 148.4, 139.8, 134.5, 126.0, 121.8, 119.0, 108.8, 103.1, 93.8, 81.9, 64.8, 61.1, 59.7, 57.7, 55.0, 46.4, 45.8, 39.4, 20.7, 19.1; HRMS (ESI): m/z Calcd. for C₂₂H₂₃FNO₇ (M+1), 432.4192; Found, 432.4196 (M+1).

### (S)-3-((R)-9-iodo-4-methoxy-6-methyl-5,6,7,8-tetrahydro-[1,3]dioxolo[4,5-g]isoquino-lin-5-yl)-6,7-dimethoxyisobenzofuran-1(3H)-one

The iodination of noscapine was achieved using pyridine-iodine chloride. Since this is not commercially available, we first prepared the said reagent using the following procedure. Iodine chloride (55 ml, 1 mol) was added to a solution of potassium chloride (120 g, 1.6 mol) in water (350 ml). The volume was then adjusted to 500 ml to give a 2 M solution. In the event the iodine chloride was under or over chlorinated, the solution was either filtered or the calculated quantity of potassium iodide added. Over chlorination was more to be avoided than under chlorination since iodine trichloride can serve as a chlorinating agent. Alternatively, the solution of potassium iododichloride was made as follows. A mixture of potassium iodate (71 g, 0.33 mol), potassium chloride (40 g, 0.53 mol) and conc. hydrochloric acid (5 ml) in water (80 ml) was stirred vigorously and treated simultaneously with potassium iodide (111 g, 0.66 mol) in water (100 ml) and with conc. hydrochloric acid (170 ml). The rate of addition of hydrochloric acid and potassium iodide solutions were regulated such that no chlorine was evolved. After addition was completed, the volume was brought to 500 ml with water to give a 2 N solution of potassium iododichloride, which itself is a very good iodinating agent. However, usage of reagent in the aromatic iodination of noscapine resulted in hydrolysis products due to the acidic nature of the reagent.

In an effort to minimize or avoid hydrolysis, a basic iodinating reagent, pyridine-iodine chloride was prepared as follows. To a stirred solution of pyridine (45 ml) in water (1 L) was added 2 M solution of potassium iododichloride (250 ml). A cream colored solid separated, the pH of the mixture was adjusted to 5.0 with pyridine and the solid collected by filtration, washed with water and air-dried to afford the pyridine-iodine chloride reagent in 97.5% yield (117 g) that was crystallized from benzene to afford light yellow solid.

Iodination of noscapine was now carried out by addition of pyridine-iodine chloride (1.46 g, 6 mmol) to a solution of noscapine (1 g, 2.42 mmol) in acetonitrile (20 ml) and the resultant mixture was stirred at room temperature for 6 hours and then at 100° C. for 6 hours. After cooling, excess ammonia was added and filtered through celite pad to remove the black nitrogen triiodide. The filtrate was made acidic with 1 M HCl and filtered to collect the yellow solid, washed with water and air-dried to afford (S)-3-((R)-9-iodo-4-methoxy-6-methyl-5,6,7,8-tetrahydro-[1,3]dioxolo[4,5-g]isoquinolin-5-yl)-6,7-dimethoxyisobenzofuran-1(3H)-one (5). Yield: 76%, mp 172.3-172.6° C.; ¹H NMR (CDCl₃, 400 MHz): δ 7.15 (d, 1H, J=8.1 Hz), 7.01 (d, 1H, J=8.1 Hz), 6.11 (s, 2H), 5.36 (d, 1H, J=4.8 Hz), 4.25 (d, 1H, J=4.8 Hz), 3.85 (s, 3H), 3.74 (s, 3H), 3.72 (s, 3H), 2.78-2.72 (m, 2H), 2.55-2.50 (m, 2H), 2.32 (s, 3H); ¹³C NMR (CDCl₃, 100 MHz): δ 168.2, 155.1, 151.5, 148.3, 146.5, 143.1, 140.3, 120.4, 119.5, 113.3, 101.5, 85.9, 82.2, 61.8, 56.6, 55.7, 54.5, 54.1, 51.2, 39.8, 30.1, 18.8; HRMS (ESI): m/z Calcd. for C₂₂H₂₃INO₇ (M+1), 540.3209; Found, 540.3227 (M+1).

HPLC Purity and Peak Attributions:

Method 1: Ultimate Plus, LC Packings, Dionex, C18 column (pep Map 100, 3 μm, 100 Å particle size, ID: 1000 μm, length: 15 cm) with solvent systems A (0.1% formic acid in water) and B (acetonitrile), a gradient starting from 100% A and 0% B to 0% A and 100% B over 25 min at a flow of 40 μL/min (Table 1).

Method 2: Ultimate Plus, LC Packings, Dionex, C18 column (pep Map 100, 3 μm, 100 Å particle size, ID: 1000 μm, length: 15 cm) with solvent systems A (0.1% formic acid in water) and B (methanol), a gradient starting from 100% A and 0% B to 0% A and 100% B over 25 min at a flow of 40 μL/min (Table 1).

Other Findings Related to Noscapine Halogenation

Aromatic halogenation constitutes one of the most important reactions in organic synthesis. Although, bromine is extensively used for carrying out electrophilic aromatic substitution reactions in the presence of iron bromide or aluminum chloride, its utility is limited because of the practical difficulty in handling this reagent in laboratories, compared to N-bromo- (NBS). Thus, NBS has proven to be a superior halogenating reagent provided benzylic bromination is suppressed. For example, Schmid reported that benzene and toluene gave nuclear brominated derivatives in good yields with NBS and AlCl₃ without solvents under long reflux using a large amount of the catalyst (>1 equiv) [30]. However, reactions using NBS in the presence of H₂SO₄, FeCl₃, and ZnCl₂ resulted in relatively low yields (21-61%) together with the polysubstituted products. In another report by Lambert et al., aromatic substituted derivatives were obtained in good yields with NBS in 50% aqueous H₂SO₄ [31], however, this method required considerably high acidic conditions which are not suitable for acid labile compounds, such as noscapine. Thus, there still exists a need to develop selective, reproducible and efficient procedures for the halogenation of such labile aromatic compounds that eliminate the limitations associated with the above discussed synthetic methods and offer quantitative yields of the desired compounds. Noscapine consists of isoquinoline and benzofuranone ring systems joined by a labile C—C chiral bond and both these ring systems contain several vulnerable methoxy groups. Thus, achieving selective halogenation at C-9 position without disruption and cleavage of these labile groups and C—C bonds was challenging. After careful titration of many conditions, simple, selective, efficient, and reproducible synthetic procedures have been developed to achieve halogenation at C-9 position. These procedures are discussed below.

First, the bromination of noscapine with bromine water in the presence of HBr was examined (Scheme 1). 9-Br-nos, (2) was prepared as described previously with minor modifications [12,32]. Noscapine (1) was dissolved in minimum amount of 48% hydrobromic acid with continuous stirring followed by the addition of freshly prepared bromine water over a period of 1 hour until the appearance of an orange precipitate. The reaction mixture was then stirred at room temperature for 1 hour to attain completion. Next, the resultant mixture was adjusted to pH 10 using ammonia solution to obtain 9-Br-nos (2) in 82% yield. Excess amount of HBr or longer reaction times were avoided because they resulted in the hydrolyzed products, meconine and cotamine. The bromination took place selectively on ring A of isoquinoline nucleus at position C-9. An absence of C-9 aromatic proton at δ 6.30-ppm in the ¹H NMR spectrum of the product confirmed bromination at C-9 position. ¹³C NMR and HRMS data support the structure of the compound.

Aromatic fluorination of noscapine was achieved by employing the fluoride form of Amberlyst-A 26, a macroreticular anion-exchange resin containing quaternary ammonium groups. The method described [33] for Hal/F exchange may also be applied to other Hal/Hal' exchange reactions. In Br/F exchange reactions, good yields were obtained only when a large molar ratio of the resin with respect to the substrate was employed. Thus, after refluxing a solution of bromonoscapine in anhydrous THF and an excess of Amberlyst-A 26 (fluorine, polymer-supported, 10 milliequivalents of dry resin; the average capacity of the resin is 4 milliequivalents per gram) for 12 hours, the resin was filtered off and the solvent was removed in vacuo to afford the desired compound (3) in 74% yield. The resin was recovered by washing with 1 N NaOH and then rinsing thoroughly with water until neutrality to generate the hydroxy-form of the resin. It was then stirred overnight with 1 N aqueous hydrofluoric acid, washed with acetone, ether and dried in a vacuum oven at 50° C. for 12 hours to afford the regenerated Amberlyst-A 26 (fluorine, polymer-supported), which can be reused.

Since iodine is the least reactive halogen towards electrophilic substitution, direct iodination of aromatic compounds with iodine presents difficulty and requires strong oxidizing conditions. Thus, a large diversity of methods for synthesis of aromatic iodides have been reported [36]. Some of these reported procedures involved harsh conditions such as nitric acid-sulfuric acid system (HNO₃/H₂SO₄), iodic acid (HIO₃) or periodic acid (HIO₄/H₂SO₄), potassium permanganate-sulfuric acid system (KMnO₄/H₂SO₄), chromia (CrO₃) in acidic solution with iodine, vanadium salts/triflic acid at 100° C., and lead acetate-acetic acid system [Pb(OAc)₄/HOAc]. N-iodosuccinimide and triflic acid (NIS/CF₃SO₃H) has also been reported for the direct iodination of highly deactivated aromatics. In addition, iodine-mercury(II) halide (I₂/HgX₂), iodine monochloride/silver sulfate/sulfuric acid system (ICl/Ag₂SO₄/H₂SO₄), N-iodosuccinimide/trifluoroacetic acid (NIS/CF₃CO₂H), iodine/silver sulfate (I₂/Ag₂SO₄), iodine/1-fluoro-4-chloromethyl-1,4-diazoniabicyclo [2.2.2]octane bis(tetrafluoroborate) (I₂/F-TEDA-BF₄), N-iodosuccinimide/acetonitrile (NIS/CH₂CN), and ferric nitrate/nitrogen tetroxide [Fe(NO₂)₃/N₂O₄] are also routinely employed for iodination. Nonetheless, iodination of noscapine even under the most gentle conditions gave only the hydrolysis products, meconine and cotamine [37]. In addition, direct aromatic iodination of noscapine using thallium trifluoroacetate or iodine monochloride also resulted in bond fission between C-5 and C-3′ under acidic conditions. Thus, different reaction conditions were tried, based upon varying pH, and found that successful introduction of the iodine atom at the desired C-9 position without disrupting other groups and bonds was stringently dependent on the acidity of the reaction media. A low acidic environment was conducive to effect iodination, whereas, higher acidity was detrimental to the iodination reaction. Thus, in this present work, two different complexes of iodine chloride were used for iodination: pyridine-iodine chloride and potassium iododichloride. Although the reaction with potassium iododichloride gave 9-I-nos (5), the yield was low and the desired product was associated with the undesirable hydrolyzed products. A suggestive reason for hydrolysis reaction could be the generation of excess amount of conc. hydrochloric acid in the reagent mixture. Since it was necessary to avoid excess acidity, excess amounts of potassium chloride were employed. Although potassium iododichloride solutions are most conveniently prepared by the addition of commercial iodine chloride to a solution of potassium chloride, it was possible to modify the procedure of Gleu and Jagemann, wherein, an iodide solution was oxidized with the calculated quantity of iodate in the presence of excess potassium chloride [38]. The pyridine-iodine chloride complex was prepared directly from pyridine and potassium iododichloride and this procedure avoided the separate isolation of the pyridine-iodine chloride-hydrogen chloride complex [39]. Thus, 9-I-nos (5) was prepared by treating a solution of noscapine in acetonitrile with pyridine-iodine chloride at room temperature for 6 hours followed by raising the temperature to 100° C. for another 6 hours. After cooling, excess ammonia was added and filtered through a celite pad to remove the black nitrogen triiodide. The filtrate was made acidic with 1 M HCl and filtered to collect the yellow solid, washed with water and air-dried to obtain the desired compound in 76% yield. A valuable advantage of this procedure lies in its applicability for the regioselective aromatic iodination of complex natural products.

Conclusions:

Relatively simple and straightforward methods for the direct, and regioselective halogenation of noscapine, which provide halogenated products in high quantitative yields, are provided herein. Although a plethora of reagents and reaction conditions have been reported for aromatic halogenation, most of them did not work well for noscapine, as it is readily hydrolysable. These synthetic strategies effect the desired transformations under mild conditions.

### Example 5

### Evaluation of the Tubulin Binding Properties of 9-Nitro-Nos

Cell Lines and Chemicals:

Cell culture reagents were obtained from Mediatech, Cellgro. CEM, a human lymphoblastoid line, and its drug-resistant variants—CEM/VLB100 and CEM/VM-1-5, were provided by Dr. William T. Beck (Cancer Center, University of Illinois at Chicago). CEM-VLB100, a multi-drug resistant line selected against vinblastine is derived from the human lymphoblastoid line, CEM and expresses high levels of 170-kd P-glycoprotein (Beck and Cirtain, 1982). CEM/VM-1-5, resistant to the epipodophyllotoxin, teniposide (VM-26), expresses a much higher amount of MRP protein than CEM cells (Morgan et al., 2000). The 1A9 cell line is a clone of the human ovarian carcinoma cell line, A2780. The paclitaxel-resistant cell line, 1A9/PTX22, was isolated as an individual clone in a single-step selection, by exposing 1A9 cells to 5 ng/ml paclitaxel in the presence of 5 μg/ml verapamil, a P-glycoprotein antagonist (Giannakakou et al., 1997). All cells were grown in RPMI-1640 medium (Mediatech, Cellgro) supplemented with 10% fetal bovine serum (Invitrogen, Carlsbad, Calif.) and 1% penicillin/streptomycin (Mediatech, Cellgro). Paclitaxel-resistant 1A9/PTX22 cell line was maintained in 15 ng/ml paclitaxel and 5 μg/ml verapamil continuously, but was cultured in drug-free medium for 7 days prior to experiment. Human fibroblast primary cultures were obtained from the Dermatology Department of the Emory Hospital, Atlanta. They were maintained in Dulbecco's Modification of Eagle's Medium 1× (DMEM) with 4.5 g/L glucose and L-glutamine (Mediatech, Cellgro) supplemented with 10% fetal bovine serum and 1% penicillin/streptomycin. Mammalian brain microtubule proteins were isolated by two cycles of polymerization and depolymerization and tubulin was separated from the microtubule binding proteins by phosphocellulose chromatography as described previously (Panda et al., 2000; Joshi and Zhou, 2001). The tubulin solution was stored at −80° C. until use.

Tubulin Binding Assay:

Fluorescence titration for determining the tubulin binding parameters was performed as described previously (Gupta and Panda, 2002). In brief, 9-nitro-nos (0-100 μM) was incubated with 2 μM tubulin in 25 mM PIPES, pH 6.8, 3 mM MgSO4 and 1 mM EGTA for 45 min at 37° C. The relative intrinsic fluorescence intensity of tubulin was then monitored in a JASCO FP-6500 spectrofluorometer (JASCO, Tokyo, Japan) using a cuvette of 0.3-cm path length, and the excitation wavelength was 295 nm. The fluorescence emission intensity of 9-nitro-nos at this excitation wavelength was negligible. A 0.3-cm path-length cuvette was used to minimize the inner filter effects caused by the absorbance of 9-nitro-nos at higher concentration ranges. In addition, the inner filter effects were corrected using a formula Fcorrected=Fobserved•antilog [(Aex+Aem)/2], where Aex is the absorbance at the excitation wavelength and Aem is the absorbance at the emission wavelength. The dissociation constant (Kd) was determined by the formula: 1/B=Kd/[free ligand]+1, where B is the fractional occupancy and [free ligand] is the concentration of free noscapine or 9-nitro-nos. The fractional occupancy (B) was determined by the formula B=ΔF/ΔFmax, where ΔF is the change in fluorescence intensity when tubulin and its ligand are in equilibrium and ΔFmax is the value of maximum fluorescence change when tubulin is completely bound with its ligand. ΔFmax was calculated by plotting 1/ΔF versus 1/ligand using total ligand concentration as the first estimate of free ligand concentration.

Tubulin Polymerization Assay:

Mammalian brain tubulin (1.0 mg/ml) was mixed with different concentrations of 9-nitro-nos (25 or 100 μM) at 0° C. in an assembly buffer (100 mM PIPES at pH 6.8, 3 mM MgSO₄, 1 mM EGTA, 1 mM GTP, and 1M sodium glutamate). Polymerization was initiated by raising the temperature to 37° C. in a water bath. The rate and extent of the polymerization reaction were monitored by light scattering at 550 nm, using a 0.3-cm path length cuvette in a JASCO FP-6500 spectrofluorometer (JASCO, Tokyo, Japan) for 30 minutes.

### Example 6

### Evaluation of Tubulin Binding Properties of Halogenated Noscapine Analogues

Cell Lines and Chemicals:

Cell culture reagents were obtained from Mediatech, Cellgro. CEM, a human lymphoblastoid line was provided by Dr. William T. Beck (Cancer Center, University of Illinois at Chicago). MCF-7 cells were maintained in Dulbecco's Modification of Eagle's Medium 1× (DMEM) with 4.5 g/L glucose and L-glutamine (Mediatech, Cellgro) supplemented with 10% fetal bovine serum (Invitrogen, Carlsbad, Calif.) and 1% penicillin/streptomycin (Mediatech, Cellgro). MDA-MB-231 and CEM cells were grown in RPMI-1640 medium supplemented with 10% fetal bovine serum, and 1% penicillin/streptomycin. Mammalian brain microtubule proteins were isolated by two cycles of polymerization and depolymerization and tubulin was separated from the microtubule binding proteins by phosphocellulose chromatography. The tubulin solution was stored at −80° C. until use.

In Vitro Cell Proliferation Assays

Sulforhodamine B (SRB) assay: The cell proliferation assay was performed in 96-well plates as described previously [12,28]. Adherent cells (MCF-7 and MDA-MB-231) were seeded in 96-well plates at a density of 5×10³ cells per well. They were treated with increasing concentrations of the halogenated analogs the next day while in log-phase growth. After 72 hours of drug treatment, cells were fixed with 50% trichloroacetic acid and stained with 0.4% sulforhodamine B dissolved in 1% acetic acid. After 30 minutes, cells were then washed with 1% acetic acid to remove the unbound dye. The protein-bound dye was extracted with 10 mM Tris base to determine the optical density at 564-nm wavelength.

MTS Assay:

Suspension cells (CEM) were seeded into 96-well plates at a density of 5×10³ cells per well and were treated with increasing concentrations of all halogenated analogs for 72 hours. Measurement of cell proliferation was performed colorimetrically by 3-(4,5-dimethylthiazol-2-yl)-5-(3-carboxymethoxyphenyl)-2-(4-sulphophenyl)-2H-tetrazolium, inner salt (MTS) assay, using the CellTiter96 AQueous One Solution Reagent (Promega, Madison, Wis.). Cells were exposed to MTS for 3 hours and absorbance was measured using a microplate reader (Molecular Devices, Sunnyvale, Calif.) at an optical density (OD) of 490 nm. The percentage of cell survival as a function of drug concentration for both the assays was then plotted to determine the IC₅₀ value, which stands for the drug concentration needed to prevent cell proliferation by 50%.

4′-6-diamidino-2-phenylindole (DAPI) Staining:

Cell morphology was evaluated by fluorescence microscopy following DAPI staining (Vectashield, Vector Labs, Inc., Burlingame, Calif.). MDA-MB-231 cells were grown on poly-L-lysine coated coverslips in 6-well plates and were treated with the halogenated analogs at 25 μM for 72 hours. After incubation, coverslips were fixed in cold methanol and washed with PBS, stained with DAPI, and mounted on slides. Images were captured using a BX60 microscope (Olympus, Tokyo, Japan) with an 8-bit camera (Dage-MTI, Michigan City, Ind.) and IP Lab software (Scanalytics, Fairfax, Va.). Apoptotic cells were identified by features characteristic of apoptosis (e.g. nuclear condensation, formation of membrane blebs and apoptotic bodies).

Tubulin Binding Assay:

Fluorescence titration for determining the tubulin binding parameters was performed as described previously [29]. In brief, 9-F-nos, 9-Cl-nos, 9-Br-nos or 9-1-nos (0-100 μM) was incubated with 2 μM tubulin in 25 mM PIPES, pH 6.8, 3 mM MgSO4, and 1 mM EGTA for 45 min at 37° C. The relative intrinsic fluorescence intensity of tubulin was then monitored in a JASCO FP-6500 spectrofluorometer (JASCO, Tokyo, Japan) using a cuvette of 0.3-cm path length, and the excitation wavelength was 295 nm. The fluorescence emission intensity of noscapine and its derivatives at this excitation wavelength was negligible. A 0.3-cm path-length cuvette was used to minimize the inner filter effects caused by the absorbance of these agents at higher concentration ranges. In addition, the inner filter effects were corrected using a formula F corrected ═F observed•antilog [(Aex+Aem)/2], where Aex is the absorbance at the excitation wavelength and Aem is the absorbance at the emission wavelength. The dissociation constant (Kd) was determined by the formula: 1/B=Kd/[free ligand]+1, where B is the fractional occupancy and [free ligand] is the concentration of 9-F-nos, 9-Cl-nos, 9-Br-nos or 9-I-nos. The fractional occupancy (B) was determined by the formula B=ΔF/ΔFmax, where AF is the change in fluorescence intensity when tubulin and its ligand are in equilibrium and ΔFmax is the value of maximum fluorescence change when tubulin is completely bound with its ligand. ΔFmax was calculated by plotting 1/ΔF versus 1/[free ligand].

Cell Cycle Analysis:

The flow cytometric evaluation of the cell cycle status was performed as described previously [12]. Briefly, 2×10⁶ cells were centrifuged, washed twice with ice-cold PBS, and fixed in 70% ethanol. Tubes containing the cell pellets were stored at 4° C. for at least 24 hours. Cells were then centrifuged at 1000×g for 10 min and the supernatant was discarded. The pellets were washed twice with 5 ml of PBS and then stained with 0.5 ml of propidium iodide (0.1% in 0.6% Triton-X in PBS) and 0.5 ml of RNase A (2 mg/ml) for 45 minutes in dark. Samples were then analyzed on a FACSCalibur flow cytometer (Beckman Coulter Inc., Fullerton, Calif.).

Immunofluorescence Microscopy:

Cells adhered to poly-L-lysine coated coverslips were treated with noscapine and its halogenated analogs (9-F-nos, 9-Cl-nos, 9-Br-nos, 9-I-nos for 0, 12, 24, 48 and 72 hours. After treatment, cells were fixed with cold (−20° C.) methanol for 5 min and then washed with phosphate-buffered saline (PBS) for 5 min. Non-specific sites were blocked by incubating with 100 μl of 2% BSA in PBS at 37° C. for 15 min. A mouse monoclonal antibody against α-tubulin (DM1A, Sigma) was diluted 1:500 in 2% BSA/PBS (100 μl) and incubated with the coverslips for 2 hours at 37° C. Cells were then washed with 2% BSA/PBS for 10 min at room temperature before incubating with a 1:200 dilution of a fluorescein-isothiocyanate (FITC)-labeled goat anti-mouse IgG antibody (Jackson ImmunoResearch, Inc., West Grove, Pa.) at 37° C. for 1 hour. Coverslips were then rinsed with 2% BSA/PBS for 10 min and incubated with propidium iodide (0.5 μg/ml) for 15 min at room temperature before they were mounted with Aquamount (Lerner Laboratories, Pittsburgh, Pa.) containing 0.01% 1,4-diazobicyclo(2,2,2)octane (DABCO, Sigma). Cells were then examined using confocal microscopy for microtubule morphology and DNA fragmentation (at least 100 cells were examined per condition). Propidium iodide staining of the nuclei was used to visualize the multinucleated and micronucleated DNA in this study.

Results and Discussion

Halogenated Noscapine Analogs have Higher Tubulin Binding Activity than Noscapine

One aspect of the analysis of the antimicrobial properties of the compounds involved determining whether the halogenated noscapine analogs bind tubulin like the parent compound, noscapine. Tubulin, like many other proteins, contains fluorescent amino acids like tryptophans and tyrosines and the intensity of the fluorescence emission is dependent upon the micro-environment around these amino acids in the folded protein. Agents that bind tubulin typically change the micro-environment and the fluorescent properties of the target protein [18,40,41]. Measuring these fluorescent changes has become a standard method for determining the binding properties of tubulin ligands including the classical compound colchicine [42]. This standard method was used to determine the dissociation constant (Kd) between tubulin and the halogenated analogs (9-F-nos, 9-Cl-nos, 9-Br-nos, and 9-I-nos). The data showed that all halogenated noscapine analogs quenched tubulin fluorescence in a concentration-dependent manner (FIG. 5A, upper panels). The dissociation constant for noscapine binding to tubulin (Kd) is 144±2.8 μM [18], 54±9.1 μM for 9-Br-nos [12] binding to tubulin and 40±8 μM for 9-Cl-nos [43] binding to tubulin. The double reciprocal plots yielded a dissociation constant (Kd) of 81±8 μM for 9-F-nos, and 22±4 μM for 5-I-nos, binding to tubulin. These results thus indicate that all halogenated analogs bind to tubulin with a greater affinity than noscapine in the following order of magnitude: 9-I-nos>9-Cl-nos>9-Br-nos>9-F-nos>Nos.

### Example 7

### Use of 9-Bromo Noscapine to Inhibit Spread of Vaccinia Virus in BSC-40 Cells

9-Bromo-noscapine was evaluated for its ability to not only bind tubilin, but to inhibit the spread of vaccinia virus in BSC-40 cells. The spread of the vaccinia virus was inhibited by binding 9-bromo-noscapine to the tubulin in the BSC-40 cells, thus inhibiting the ability of the vaccinia virus to transport itself across the microtubulin structure within the cells.

Plaque assays of vaccinia virus in BSC-40 cells infected and left untreated (control) or treated with DMSO (0.1% carrier) or 25 uM Br-Noscopine in 0.1% DMSO are shown in FIG. 1. Clear areas in control and DMSO treated monolayers represent areas where infected cells have lysed.

It is important to note that 9-bromo-noscopine does not prevent infection, but only small “pinpoint” plaques are evident. Pinpoint plaques indicate that the virus does not spread from cell to cell, and are consistent with inhibition of microtubule transit, which allows the virus to move to the periphery of an infected cell. Without movement, virus spreads less quickly, and smaller plaques result.

### Example 8

### Methods for Determining Activity of Compounds at Inhibiting Tubulin Binding

In order to determine the efficacy of the noscapine analogs described herein at inhibiting viral intracellular transport, and, therefore, viral replication, one can perform imaging experiments using cells to be infected, viruses to infect the cells, and the presence or absence of putative active agents to disrupt the transportation of the viruses through the cells.

For example, one can visualize how a putative active agent effects the movement of viruses by tracking the cytoplasmic movement of viruses. This can be done, for example, by tagging the virus with chemical fluorophores, followed by imaging in living cells using wide field fluorescence microscopy.

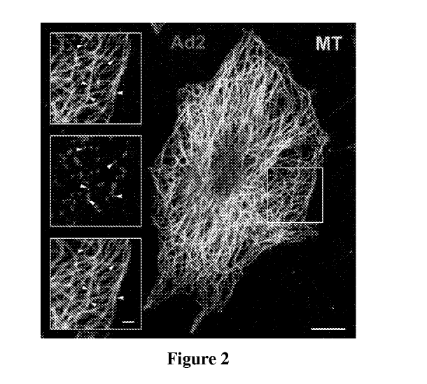

One paper, Suomalainen, et al., Adenovirus-activated PKA and p38/MAPK pathways boost microtubule-mediated nuclear targeting of virus,” EMBO J. 20, 1310-1319 (2001), shows such a fluorescent assay. For example, FIG. 2 shows adenoviruses associated with the microtubules moving toward and away from the microtubule-organizing center of the cell (MTOC). Adenoviruses tagged with a few fluorophores on each of the 252 copies of the capsid hexon trimer were fully infectious and associated with microtubules (see FIG. 3).

Imaging cells during the establishment of infection reveals that fluorescent capsids move in a microtubule-dependent fashion both toward and away from the MTOC at speeds of 1-3 μm/s.

Additionally, advances in fluorophore technology, including advances in fluorophore stability, quantum yields, new GFP variants, and more sensitive cameras have made it relatively straightforward to image the motility of many different fluorescently tagged viruses with good temporal resolution. For example, it has become possible to image adeno-associated virus (AAV) type 2, a small parvovirus which can accept only a few fluorophores in its 20 nm sized capsid without loosing infectivity, at 25 frames per second, for periods of a few seconds. (Seisenberger, G., Ried, M. U., Endress, T., Buning, H., Hallek, M., and Brauchle, C. (2001). Real-time single-molecule imaging of the infection pathway of an adeno-associated virus. Science 294, 1929-1932.)

One example of the type of assay that can be performed involves taking a photograph of a confocal laser scanning microscope image, where viral particles (such as adenovirus Type 2 particles) are associated with a cell (such as a HeLa cell). For example, FIG. 2 shows that incoming adenovirus type 2 particles are associated with microtubules. A single 120 nm optical section from a confocal laser scanning microscope showing the microtubule cytoskeleton (green) of a HeLa cell was infected with Texas red-labeled Ad2 particles (red) for 30 minutes. Enlarged insets highlight the colocalization of Ad2 particles (arrowheads) with microtubules in the periphery of the cell. The bars in the photograph are 10 mm and 2 mm, respectively. Using this approach, putative active compounds can be incubated with the HeLa cells, and the fluorescently-labeled virus particles can be used to infect the incubated cells. The resulting confocal laser scanning microscope image can be taken and compared with control to show the degree to which microtubule binding was inhibited.

Another method for monitoring the efficacy of a compound to affect the virus-cytoskeletal interaction is to construct an in vitro assay system to study the microtubule-dependent viral movement. For example, an optical microchamber designed to monitor microtubule-based endosomal traffic in vitro can be constructed, containing pre-bound rhodamine-labeled microtubules and GFP-tagged viruses. The viruses can be associated with cellular structures in this assay system, and can include fully-enveloped capsids within organelles and capsids associated with the surface of organelles.

The movement of the virus-organelle structure can be monitored with and without the addition of the compound to determine the efficacy of the compound in disrupting the movement of the virus along the cytoskeletal system.

This type of assay system has been used to elucidate the movement of Herpes Simplex viruses (HSV) and the principal site of HSV envelopment and egress within the cell (Lee, Grace E., Murray, John W., Wolkoff, Allan W., and Wilson, Duncan W., (2006) Reconstitution of Herpes Simplex Virus Microtubule-Dependent Trafficking In Vitro, Journal of Virology, May 2006, p. 4264-4275). A representative graph from this paper is shown in FIG. 3, which shows membrane-associated cytoplasmic HSV capsids bound to microtubules in vitro.

In FIG. 3A, bouyant organelles were isolated from the cytoplasm of HSV K26GFP-infected cells. They then flowed into an imaging chamber, which contained pre-bound rhodamine-labeled microtubules. After an incubation of 5 to 10 min, unbound material was washed away, and the chamber was imaged using fluorescence microscopy. The upper panel shows microtubules in red, and bound HSV-containing organelles in green. The lower panel is another representative field shown in black and white. Scale bar, 10 nm. In FIG. 3B, HSV was bound to microtubules as in FIG. 3A, and the chamber was then fixed in glutaraldehyde and prepared for transmission electron microscopy as described. This representative image appears to show HSV capsids partially or completely enclosed by an organelle (arrowhead) or adjacent to an organelle (black arrow) and in both cases attached to a microtubule (white arrow). The scale bar represents 100 nM.

The above-described method can also be used in conjunction with putative active agents to determine their efficacy. The cells can be incubated with the putative active agents, at varying concentrations and for varying times, and their ability to inhibit microtubulin binding can be assayed by evaluating the binding of the rhodamine-labeled viruses.

Additional synthetic examples relate to the preparation of compounds of Formula V.

### Example 1

### 3-(9-Fluoro-4-methoxy-6-methyl-5,6,7,8-tetrahydro 1,3 dioxolo-4,5-g isoquinoline-5-yl)-6,7-dimethoxy-3H-isobenzofuran-1-one

To the solution 1.03 g (2.5 millimole) of NSC in 10 ml Of AcOH they add 0.61 g (2.5 millimole) of the solution of HBr into AcOH, after which add dropwise the solution 0.8 g (5 millimole) of bromine in 2 ml Of AcOH (after the addition of HBr possibly the formation of sediment of hydrobromide NSC3 which on the motion of bromination is dissolved). After 15 min of mixing the reaction mixture pours out on 60 ml of cooled to OC saturated solutions ammonia. They filter the fallen colorless sediment, they wash thoroughly in water, dry, obtain 63% of A-Ol. NMR-1H (CDCl₃, TMS): d 7.03 (Jo=8.4 Hz, IH, 5-H), d 6.30 (Jo=8.4 Hz, IH, 4-H), with 6.02 (2H, 2′-H), d 5.49 (J=4.8 Hz, IH, 3-H), d 4.33 (J=4.8 Hz, IH5 of 5′-H), s 4.09 (ZN, OCH3), s 3.98 (ZN, OCH3), s 3.88 (ZN, OCH3), m 2.6-2.8 (2H, 7′-H), s 2.51 (ZN, 6′-CH3), m. 2.42-2.50 (IH, 8′-H), m 1.92-2.01 (IH, 8′-H); NMR-13C (CDCl3, TMS): 168.03, 152.36, 147.83, 146.58, 141.32, 140.02, 134.22, 130.38, 119.69, 119.05, 118.42, 117.53, 101.10, 95.61, 81.32, 62.32, 60.97, 59.46, 56.85, 48.46, 45.23, 25.96.

### Example 2

### 3-(9-Iodo-4-methoxy-6-methyl-5,6,7,8-tetrahydro 1, 3 Dioxolo-4,5-g isoquinoline-5-yl)-6,7-dimethoxy-3H-isobenzofuran-1-one (1)

The solution 206 mH (0.5 millimole) of NSC in 4 ml Of acOH is mixed up with 100 mH (0.6 millimole) of ICl and are intermixed 3 h with 500 C (control of reaction with the aid of the LC-Ms). The reaction mixture is neutralized with ammonia during the cooling with ice. The sediment is filtered, washed in water, and dried. Are obtained 246 mg (71%) 1 (1). 1H NMR (400 MHz, CDCl₃, TMS): δ 7.02 (d, J=8.4 Hz, IH), 6.27 (d, J=8.4 Hz, IH), 6.01 (s, 2H), 5.48 (d, IH, IH, J=4.0 Hz), 4.32 (d, IH, J=4.0 Hz), 4.10 (s, ZN), 3.99 (s, ZN), 3.88 (s, ZN), 2.65-2.74 (m, IH), 2.51 (s, ZN), 2.42-2.62 (m, 2H), 1.89-1.96 (m, IH), 13C NMR (100 MHz, CDCl3, TMS): 6 168.01, 152.35, 149.89, 147.85, 141.35, 140.95, 133.08, 133.03, 119.74, 119.39, 118.37, 117.54, 100.28, 81.34, 69.32, 62.33, 61.12, 59.48, 56.87, 49.13, 45.25, 30.97.

### Example 3

### 3-(9-Chloromethyl-4-methoxy-6-methyl-5,6,7,8-tetrahydro 1,3-di-oxolo 4,5-g isoquinoline-5-yl)-6,7-dimethoxy-3H-isobenzofuran-1-one hydrochloride 1(2)

To the solution 1.11 g (2.5 millimole) of 5-HOCH₂—NSC A-04 in 10 ml of dichloromethane they add dropwise the solution 0.45 g (0.27 ml, 3.75 millimole) Of SOCl₂ in 3 ml of dichloromethane, supporting the temperature of reaction mixture in the interval 0-3 S. Then they intermix at this temperature for 20 min, they after which give to it to be heated to room temperature is intermixed 2.5 additional h. Solvent is removed on the rotary vaporizer at a temperature not higher than 200 C, remainder is dissolved in acetone they will re-precipitate by ether, they are maintained several hours in the refrigerator, they filter the solid hygroscopic substance, which is used in further syntheses without the additional cleaning. Are obtained 1.18 g (95%) 1 (2). NMR-1H (CDCl3, TMS): d 7.65 (Jo=8.3 Hz, IH, 5-H), d 7.30 (Jo=8.3 Hz, IH, 4-H), br.s (IH, 3-H), d 5.95 (J=I.5 Hz, IH, 2′-H), d. 5.89 (J=1.5 Hz, IH3 of 2′-H), br.s. 5.22 (IH, 5′-H), d 4.64 (J=I LO Hz, IH, 9-CH2—Cl), d 4.49 (J=I LO Hz, IH, 9-CH2-Cl), m 4.10-4.21 (IH, 7′-H), s 3.98 (ZN, OCH3), s 3.91 (ZN, OCH3), m 3.44-3.53 (IH, 7′-H), s 3.26 (ZN, OCH3), m 3.10-3.30 (2H, 8′-H), br.s 2.88 (ZN, 6′-CH3); NMR-13C (CDCl3, TMS): 166.52, 152.79, 149.48, 147.69, 14.27, 139.20, 133.29, 125.92, 119.57, 118.90, 117.12, 110.63, 107.63, 101.72, 78.61, 62.21, 62.17, 58.47, 56.99, 44.76, 39.89, 36.69, 18.19.

### Example 4

### 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carbaldehyde 1 (3)

Is mixed up the solution 200 mH (0.4 millimole) of hydrochloride 1 (2) in 2 ml of water with 0.6 ml 1N NaOH. They extract the obtained emulsion by chloroform, extract is dried by the waterless Na₂SO₄, they concentrate to 2 ml and then boiled with 70 mH (0.5 millimole) of hexamethylentetramine. Reaction mass is cooled, they filter sediment, they wash in ether, dry and dissolve in 3 ml of water. The obtained solution they boil 2 h, cool and they extract by chloroform. Extract is dried by the waterless Na₂SO₄, they concentrate and i-RrON— ether is mixed up with the mixture. Sediment separate, they dry they obtain 1 (3) in the form hydrochloride, NMR-1H (400 MHz, CDCl3): 10.21 (IH, s); 7.75 (IH, d, J=7.6 Hz); 7.29 (IH, d, J=7.6 Hz); 6.58 (IH, br.m), 6.04 (IH, s), 6.00 (IH, s), 5.29 (IH, br. M), 4.01-4.12 (IH, t), 3.99 (ZN, s), 3.91 (ZN, s); 3.67-3.75 (IH, t); 3.42-3.52 (IH, t); 3.31-3.40 (IH, t); 3.35 (ZN, s); 2.91 (ZN, s); 2.91 (ZN, s); 1.84 (br.s); Hydrochloride 1 (3) then dissolve in the water, neutralize by aqueous ammonia, sediment they filter they dry and are obtained by 102 mH (46%) 1 (3), 1H NMR (400 Hz, CDCl3): 10.26 (Sh, s); 7.05 (IH, d, J=8.4 Hz); 6.51 (IH, d, J=8.4 Hz); 6.08 (2H, s); 5.43 (IH, d, 5.1 Hz), 4.29 (IH, d, J=5.1 Hz); 4.10 (ZN, s); 4.08 (ZN, s); 3.89 (ZN, s); 3.13-3.19 (IH, m); 2.82-2.87 (IH, m); 2.51 (ZN, s); 2.39-2.50 (2H, m), 13C NMR (100 MHz, CDCl3): 187.2; 168.0; 153.3; 152.4; 147.9; 145.1; 141.7; 133.5; 133.3; 119.5; 118.6; 118.1; 117.4; 111.4; 102.1; 81.2; 62.3; 61.0; 59.6; 56.8; 47.8; 45.1; 23.4.

### Example 5

Method of obtaining is 5-(4,5-dimethoxy-3-oxo-1,3-dihydroisobenzofuran-1-yl)-4-methoxy-6-methyl-5,6,7,8-tetrahydro-1,3 dioxolo 4,5-g isoquinoline-9-carboxylic acid 1 (4). The mixture 0.2 millimole 3-(9-From-4-methoxy-6-methyl-5,6,7,8-tetrahydro 1,3 dioxolo-4,5-g isoquinoline-5-yl)-6,7-dimethoxy-3H-isobenzofuran-1-on A-ol or 3-(9-iodo-4-methoxy-6-methyl-5,6,7,8-tetrahydro 1,3 dioxolo-4,5-g isoquinoline-5-yl)-6,7-dimethoxy-3H-isobenzofuran-1-on 1 (1), 36 mH (0.4 millimole) cuCN and 2 ml dimethylformamide intermix 24 h with 130° C. in the inert atmosphere. Reaction mass they cool to 40° C. and add during mixing 15 ml of ammonia and 15 ml chloroform. Organic layer they separate, wash in water, dry above Na₂SO₄, then filter, the obtained solution intermix 15 min with activated carbon, then filter and concentrate. They filter and recrystallize sediment from isopropanol. Obtain 1 (4), 1H NMR (400 MHz, CDCl3, TMS): 7.06 (d, J=8.1 Hz, IH, 5-H), 6.46 (d, J=8.1 Hz, IH), 6.06 (d, IH, J=I.1 Hz), 6.05 (d, IH, J=L1 Hz), 5.42 (d, J=4.8 Hz, IH), 4.23 (d, J=4.8 Hz, IH), 4.09 (s, 3H), 4.04 (s, 3H), 3.88 (s, 3H), 2.73-2.87 (m, 2H), 2.52-2.55 (m, IH), 2.50 (s, 3H), 2.18-2.24 (m, IH); 13C NMR (100 MHz, CDCl3): 168.0; 152.7; 152.3; 148.2; 144.5; 141.5; 133.9; 133.7; 119.6; 118.9; 118.8; 117.5; 114.0; 102.4; 87.3; 81.0; 62.5; 61.0; 59.8; 57.0; 47.7; 45.1; 24.6.

### Example 6

3-(9-Methoxymethyl-4-methoxy-6-methyl-5,6,7,8-tetrahydro 1,3-di-oxolo 4,5-g isoquinoline-5-yl)-6,7-dimethoxy-3H-isobenzofuran-1-on 1 (5). To the suspension 100 mH (0.2 millimole) 1 (2) in 3 ml MeOH are added 0.5 ml of diisopropylethylamine, mixture boils before the complete dissolution of initial hydrochloride, they cool, they process by water, fallen oil they extract EtOAc, organic layer dries above Na₂SO₄, is steamed solvent, remainder cleans flesh—by chromatography (hexane—EtOAc from 40 to 60%), are obtained by 69 mH (75%) 1 (5) in the form the oil-like slowly crystallizing substance. H NMR (CDCl3, TMS): d 6.94 (Jo=8.4 Hz, IH, 5-H), d 6.14 (Jo=8.4 Hz, IH, 4-H), with 5.96 (2H, 2′-H), d 5.53 (J=4.8 Hz, IH, 3-H), d 4.39 (J=4.8 Hz, IH, 5′-H), s 4.39 (2H, 9-CH2-), s 4.09 (ZN, OCH3), s 4.02 (ZN, OCH3), s 3.85 (ZN, OCH3), s 3.34 (2H, OCH3), m 2.56-2.72 (2H, 7′-H), s 2.53 (ZN, 6′-CH3), m 2.33-2.40 (IH, 8′-H), m 1.84-1.96 (IH, 8′-H); 13C NMR (CDCl3, TMS): 168.16, 152.23, 147.98, 147.72, 141.33, 140.38, 133.23, 132.27, 120.08, 118.24, 117.77, 117.57, 110.38, 100.87, 81.82, 65.03, 62.31, 61.07, 59.40, 57.78, 56.83, 49.50, 46.06, 23.58.

### Example 7

### General Method of Obtaining 3-(9-aryl-4-methoxy-6-methyl-5,6,7,8-tetrahydro 1,3 dioxolo-4,5-g isoquinoline-5-yl)-6,7-dimethoxy-3H-isobenzofuran-1-one of general Formula 1.1.

Mixture 246 mH (0.5 mole) A-ol, 0.6 millimole of arylboronic acid 2, 70 mH (0.1 millimole) of PdO₂ (PPh₃) 2, 652 mH (2 millimole) Of CS₂ (CO₃)₂ in 5 ml of degassed DME heat in the microwave furnace with 140 with 30 min, reaction mixture they filter through it settles, washes sediment on the filter DME, the united filtrate is steamed dry, the remainder several times process 2N HCl, every time leading to the easy boiling during the mixing and decanting aqueous layer from the resinous remainder. To the united aqueous solution they add activated carbon, lead to the boiling, they filter by the hot through it settles, they cool filtrate and process by the surplus of the aqueous solution NH₃.

After maintaining of mixture in the refrigerator for 2-3 hours, they filter the fallen sediment, they wash in the large number of water, obtain 160 mg colorless product 1.1. If necessary substance can be additionally purified by flesh-chromatography (hexane—gradient of ethyl acetate of 60 to 80%), including: 3-(9-phenyl-4-methoxy-6-methyl-5,6,7,8-tetrahydro 1,3 dioxolo-4,5-g isoquinoline-5-yl)-6,7-dimethoxy-3H-isobenzofuran 1.1 (1), NMR-1H (CDCl3, TMS): m 7.38-7.44 (2H, Ar—H), m 7.31-7.36 (IH, Ar—H), rn7.23-7.26 (2H, Ar—H), d 7.02 (Jo=8.4 Hz, IH, 5-H), d 6.13 (Jo=8.4 Hz, IH, 4-H), d 5.98 (J=I.5 Hz, 2′-H), d 5.92 (J=I.5 Hz, 2′-H), d 5.55 (J=4.8 Hz, IH, 3-H), d 4.51 (J=4.8 Hz, IH, 5′-H), s 4.11 (ZN, OCH3), s 4.10 (ZN, OCH3), s 3.90 (ZN, OCH3), m 2.57-2.61 (IH, 7′-H), s 2.56 (ZN, 6′-CH3), m 2.14-2.25 (2H, 7′H, 8′-H), m 1.62-1.731 (IH, 8′-H), NMR-13C (CDCl3, TMS): 168.10, 152.34, 147.74, 146.06, 141.04, 139.71, 134.23, 133.81, 130.90, 130.02, 128.26, 127.478, 120.60, 117.95, 117.88, 116.57, 100.88, 82.05, 62.33, 61.19, 59.58, 56.97, 50.97, 46.85, 27.14; 3-9(4-methoxyphenyl)-4-methoxy-6-methyl-5,6,7,8-tetrahydro-1,3 dioxolo-4,5-g isoquinoline-5-yl-6,7-dimethoxy-3H-isobenzofuranyl 1.1 (3), 1H NMR (400 MHz, CDCl3, TMS): D 7.17 (d, 2H, J=8.8 Hz), 7.00 (d, J=8.1 Hz, IH), 6.95 (d, 2H, J=8.8 Hz), 6.11 (d, IH, J=8.1 Hz,), 5.98 (d, IH, J=L1 Hz), 5.91 (d, IH, J=L1 Hz), 5.55 (d, IH, J=4.0 Hz), 4.49 (d, IH, J=4.0 Hz), 4.11 (s, ZN), 4.03 (s, ZN), 3.90 (s, ZN), 3.84 (s, ZN), 2.65-2.70 (m, IH), 2.56 (s, ZN), 2.15-2.30 (m, 2H), 1.66-1.73 (m, IH); 3-9 (3-pyridyl)-4-methoxy-6-methyl-5,6,7,8-tetrahydro 1,3-dioxolo 4,5-g isoquinoline-5-yl-6,7-dimethoxy-3H-isobenzofuran-1-one 1.1 (17), NMR-1H (CDCl3, TMS): dd 8.58 (J=4.8 Hz, J=I.5 Hz, IH, Py-H), d 8.50 (J=I.5 Hz, IH, Py-H), m 7.60-7.64 (IH, Py-H), m 7.33-7.38 (IH, Py-H), d 7.03 (Jo=8.4 Hz, IH, 5-H), d 6.18 (J=8.4 Hz, IH, 4-H), d 6.00 (J=I.5 Hz, 2′-H), d 5.93 (J=I.5 Hz, 2′-H), d 5.54 (J=4.8 Hz, IH, 3-H), d 4.50 (J=4.8 Hz, IH, 5′-H), s 4.11 (6H, OCH3), s 3.91 (ZN, OCH3), m 2.59-2.65 (IH, 7′-H), s 2.56 (ZN, 6′-CH3), m 2.16-2.24 (2H, 7′H, 8′-H), m 1.69-1.77 (IH, 8′-H), NMR-13C (CDCl3, TMS): 167.99, 152.44, 150.86, 148.49, 147.81, 146.51, 140.93, 140.35, 137.41, 133.83, 130.84, 130.29, 123.17, 120.53, 118.34, 117.83, 117.67, 112.66, 101.03, 81.92, 62.33, 61.17, 59.58, 56.95, 50.75, 46.47, 26.98; 3-9 (4 pyridyl)-4-methoxy-6-methyl-5,6,7,8-tetrahydro 1, 3-di-oxolo 4,5-g isoquinoline-5-yl-6,7-dimethoxy-3H-isobenzofuran-1-one 1.1 (18): NMR-1H (CDCl3, TMS): d 8.64 (J=5.9 Hz, 2H, Py-H), d 7.19 (J=5.9 Hz, 2H, Py-H), d 7.03 (Jo=8.4 Hz, IH, 5-H) 3d 6.20 (Jo=8.4 Hz, IH, 4-H), d 6.01 (J=I.5 Hz, 2′-H), d 5.94 (J=I.5 Hz, 2′-H), d 5.53 (J=4.8 Hz, IH, 3-H), d 4.48 (J=4.8 Hz, IH, 5′-H), s 4.12 (ZN, OCH3), s 4.10 (ZN, OCH3), s 3.91 (ZN, OCH3), m 2.61-2.69 (IH, 7′-H) 5 s 2.56 (ZN, 6′-CH3), m 2.16-2.28 (2H, 7′H, 8′-H), m 1.72-1.83 (IH, 8′-H); NMR-13C (CDCl3, TMS): 167.96, 152.44, 149.75, 147.87, 146.20, 142.47, 141.03, 140.60, 133.84, 130.44, 124.99, 120.50, 118.48, 117.89, 117.68, 113.61, 101.09, 81.84, 62.35, 61.17, 59.58, 56.99, 50.62, 46.64, 26.97; tetrahydro 1,3-di-oxolo 4,5-g isoquinoline-5-yl-6,7-dimethoxy-3H-isobenzofuran-1-yl 1.1 (24), 1H NMR (400 MHz, CDCl3, TMS): D 9.17 (sDN), 8.68 (s, 2H), 7.03 (d, J=8.4 Hz, IH), 6.22 (d, J=8.4 Hz, IH), 6.03 (d, J=I.1 Hz), 5.96 (d, J=L1 Hz), 5.52 (d, J=4.1 Hz, IH), 4.49 (d, J=4.1 Hz, IH), 4.11 (s, 6H), 3.91 (s, 3H), 2.65-2.70 (m, IH), 2.57 (s, 3H), 2.17-2.27 (m, 2H), 1.77-1.85 (m, IH); 4 (5R)-5-(1S)-(4,5-dimethoxy-3-oxo-1,3-dihydro-2-benzofuran-yl-4-methoxy-6-methyl-5,6,7,8-tetrahydro-1,3 dioxolo 4,5-g isoquinoline-9-benzocarboxamide 1.1 (25), 1H NMR (400 MHz, CDCl3, TMS): D 7.86 (d, 2H, J=8.4 Hz), 7.35 (d, 2H, J=8.4 Hz), 7.03 (d, J=8.0 Hz, IH), 6.20 (d, J=8.0 Hz, IH), 5.59 (br.s, IH), 5.99 (d, IH, J=1.5 Hz), 5.93 (d, IH, J=1.5 Hz), 5.75 (br.s, IH), 5.54 (d, IH, J=4.0 Hz, IH), 4.50 (d, IH, J=4.0 Hz), 4.11 (s, 3H), 4.09 (s, 3H), 3.91 (s, 3H), 2.65-2.70 (m, IH), 2.56 (s, 3H), 2.17-2.25 (m, 2H), 1.70-1.78 (m, IH), etc 3-(9-aryl-4-methoxy-6-methyl-5,6,7,8-tetrahydro 1,3 dioxolo-4,5-g isoquinoline-5-yl)-6,7-dimethoxy-3H-isobenzofuran-1-yl 1.1, whose combinatory library is represented in Table 2.

TABLE 2
Combinatorial library of 3-(9-aryl-4-methoxy-6-methyl-5,6,7,8-tetrahydro 1,3
dioxolo-4,5-g isoquinoline-5-yl)-6,7-dimethoxy-3H-isobenzofuran-1-one 1.1

| | | | LC-MS, |
|---|---|---|---|
| | | [symbol] | m/z |
| [symbol] | [symbol] | (M) | (M + H) |
| 1.1(1) | 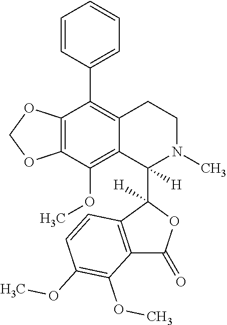 | 489.53 | 490 |

[symbol]

2

| | | | |
|---|---|---|---|
| 1.1(2) | 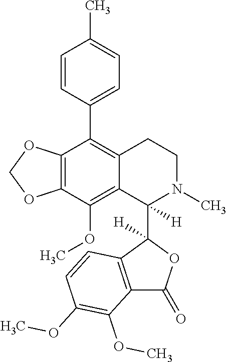 | 503.56 | 594 |
| 1.1(3) | 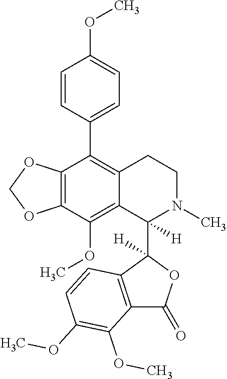 | 519.56 | 520 |
| 1.1(4) | 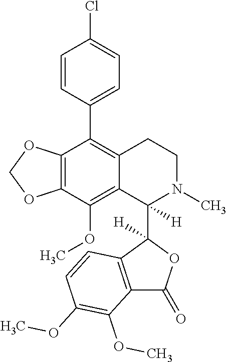 | 523.97 | 524 |
| 1.1(5) | 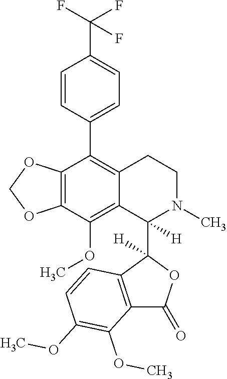 | 557.53 | 558 |
| 1.1(6) | 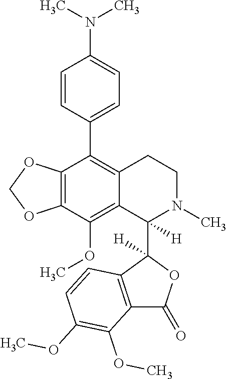 | 532.60 | 533 |
| 1.1(7) | 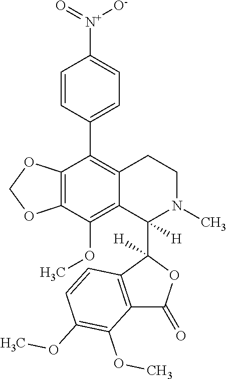 | 534.53 | 535 |
| 1.1(8) | 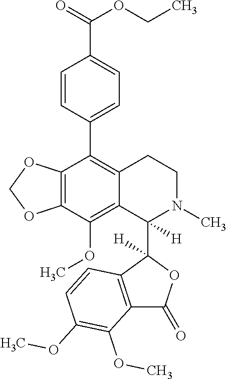 | 561.59 | 562 |
| 1.1(9) | 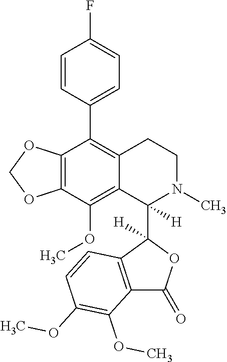 | 507.52 | 508 |
| 1.1(10) | 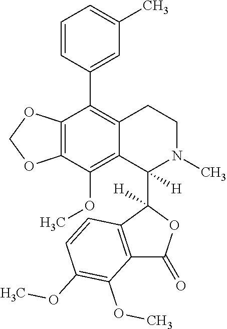 | 503.56 | 504 |
| 1.1(11) | 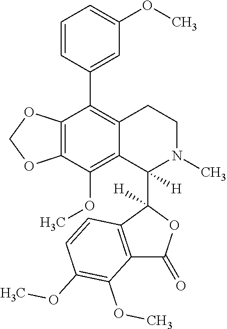 | 519.56 | 520 |
| 1.1(12) | 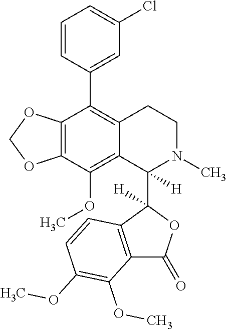 | 523.97 | 524 |
| 1.1(13) | 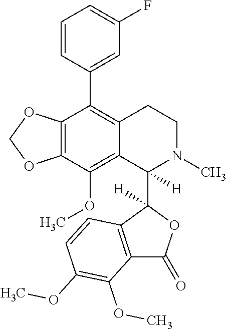 | 507.52 | 508 |
| 1.1(14) | 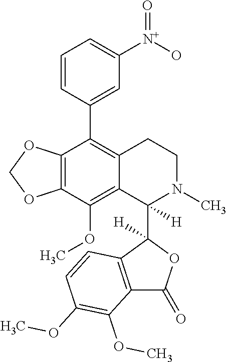 | 534.53 | 535 |
| 1.1(15) | 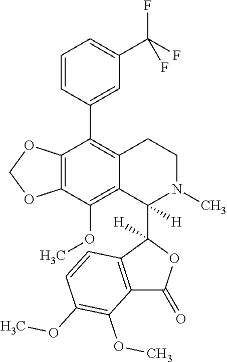 | 557.53 | 558 |
| 1.1(16) | 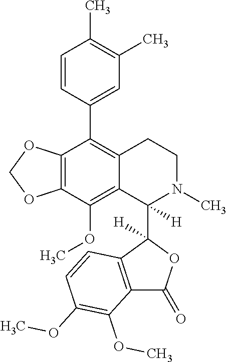 | 517.58 | 518 |
| 1.1(17) | 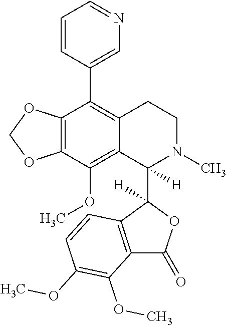 | 490.52 | 491 |
| 1.1(18) | 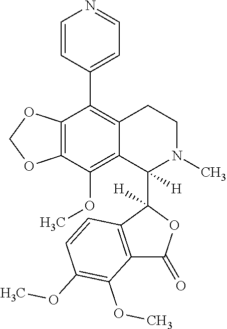 | 490.52 | 491 |
| 1.1(19) | 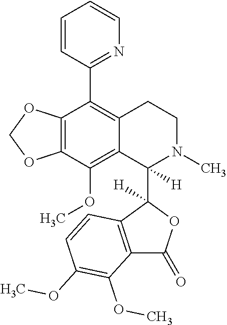 | 490.52 | 491 |
| 1.1(20) | 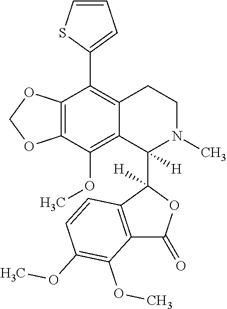 | 495.56 | 496 |
| 1.1(21) | 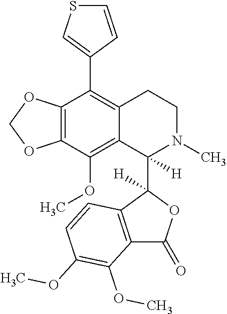 | 495.56 | 496 |
| 1.1(22) | 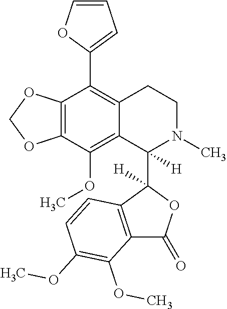 | 479.49 | 480 |
| 1.1(23) | 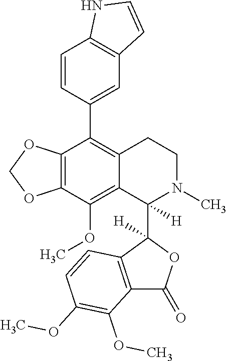 | 528.57 | 529 |
| 1.1(24) | 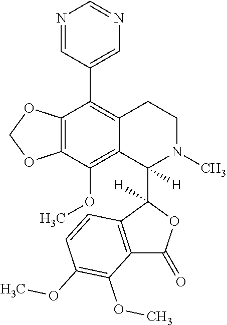 | 491.51 | 492 |
| 1.1(25) | 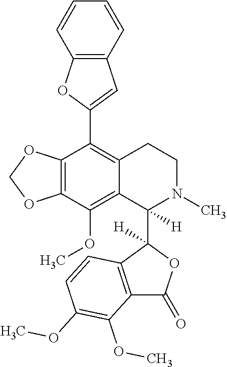 | 529.55 | 530 |
| 1.1(26) | 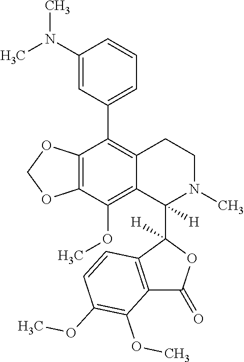 | 532.60 | 533 |
| 1.1(27) | 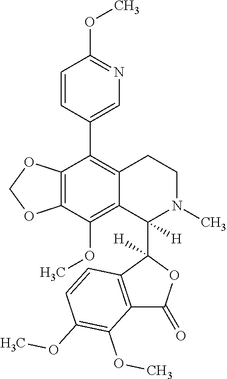 | 520.54 | 521 |
| 1.1(28) | 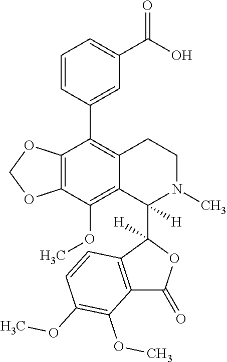 | 533.54 | 534 |
| 1.1(29) | 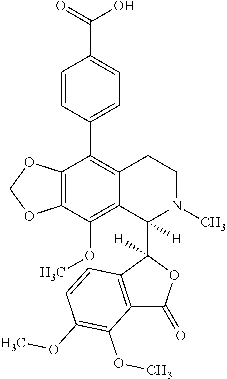 | 533.54 | 534 |
| 1.1(30) | 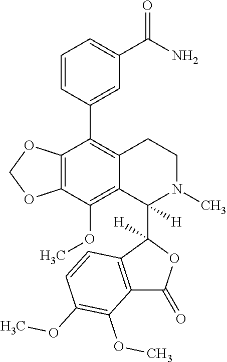 | 532.56 | 533 |
| 1.1(31) | 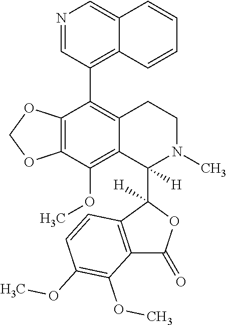 | 540.58 | 541 |
| 1.1(32) | 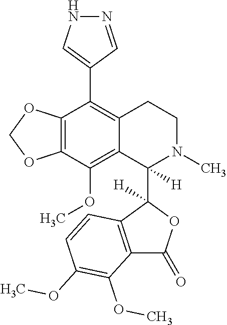 | 479.49 | 480 |
| 1.1(33) | 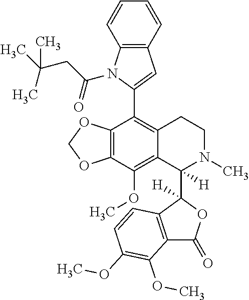 | 628.69 | 629 |
| 1.1(34) | 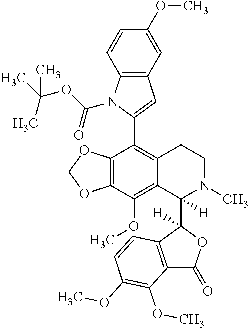 | 658.71 | 659 |
| 1.1(35) | 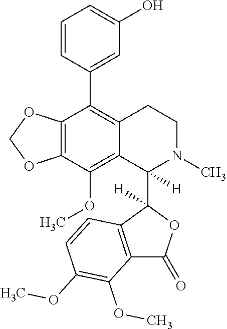 | 505.53 | 506 |
| 1.1(36) | 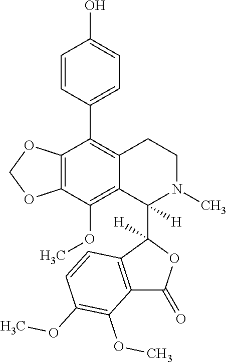 | 505.53 | 506 |
| 1.1(37) | 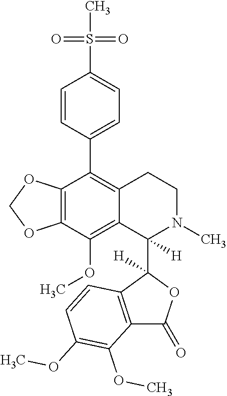 | 567.62 | 568 |
| 1.1(38) | 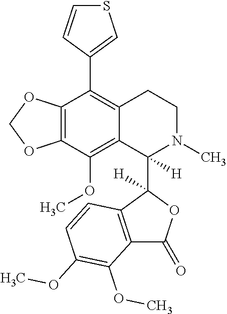 | 495.56 | 496 |
| 1.1(39) | 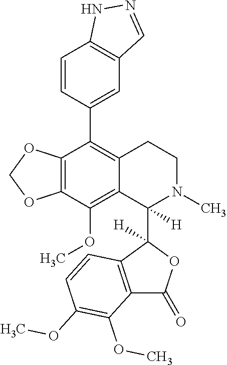 | 529.55 | 530 |
| 1.1(40) | 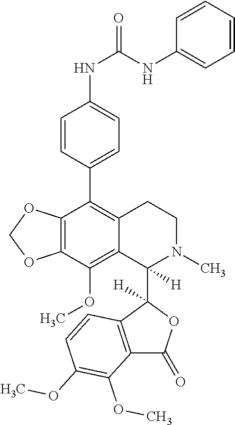 | 623.67 | 624 |
| 1.1(41) | 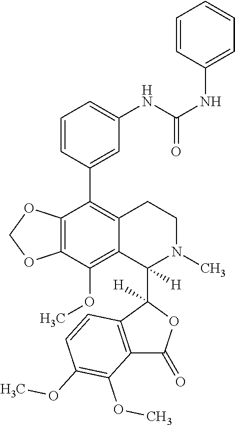 | 623.67 | 624 |

### Example 7

Method of obtaining 3-(9-aminomethyl-4-methoxy-6-methyl-5,6,7,8-tetrahydro-1,3 dioxolo 4,5-g isoquinoline-5-yl)-6,7-dimethoxy-3H-isobenzo-furan-1-one 1.2. To the solution 1 ml of amine in 3 ml of MeOH add 100 mL (0.2 millimole) 1 (2), mixture lead to the boiling, cool, process by water, they extract by ethyl acetate, organic layer they dry above Na₂SO₄, steam solvent, remainder clean flesh—by chromatography (20% gekcana into EtOAc—clean EtOAc), they obtain 1.2, including: 3-(9 N-morpholinomethyl-4-methoxy-6-methyl-5,6,7,8-tetrahydro 1, 3 dioxolo 4,5-g isoquinoline-5-yl)-6,7-dimethoxy-3H-isobenzofuran-1-one 1.2 (5), NMR-1H (CDCl3, TMS): d 6.90 (Jo=8.1 Hz, Sh, 5-H), d 6.11 (Jo=8.1 Hz, IH, 4-H), d 5.94 (J=1.5 Hz, IH, 2′-H), d 5.92 (J=I.5 Hz, IH, 2′-H), d 5.54 (J=4.4 Hz, IH, 3-H), d 4.40 (J=4.4 Of hz5 IH, of 5′-H), s 4.10 (3H, OCH3), s 4.01 (ZN, OCH3), s 3.86 (ZN, OCH3), m 3.65-3.69 (4H, CH2N—CH2), d 3.42 (J=12.5 Hz, IH, 9′-CH) 5 d 3.37 (J=12.5 Hz, IH, 9′-CH), m 2.64-2.72 (2H, 7′-H) 5 s 2.54 (ZN, 6′-CH3), m 2.40-2.46 (4H, CH2—N —CH2), m 2.31-2.39 (IH, 8′-H), m 1.88-1.98 (IH, 8′-H); NMR-13C (CDCl3, TMS): 168.11, 152.20, 148.10, 147.792, 141.47, 139.698, 132.99, 132.594, 120.17, 118.05, 117.67, 117.52, 110.23, 100.61, 81.88, 67.19, 62.32, 61.08, 59.38, 56.85, 53.26, 52.99, 49.90, 46.27, 24.16, etc 3-(9-aminomethyl-4-methoxy-6-methyl-5,6,7,8- of tetrahydro-1,3 dioxolo 4,5-g isoquinoline-5-yl)-6,7-dimethoxy-3H-isobenzofuranyl are that 1.2, whose combinatory library is represented in Table 3.

TABLE 3
combinatory library 3-(9-aminomethyl-4-methoxy-6-methyl-5,6,7,8-
tetrahydro-1,3-dioxolo 4,5-g isoquinoline-5-yl)-6,7-dimethoxy-3H-
isobenzofuran-1-one 1.2

| | | [symbol] | LC-MS, |
|---|---|---|---|
| [symbol] | [symbol] | (M) | m/z (M + H) |
| 1.2(1) | 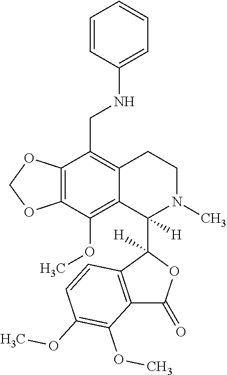 | 518.57 | 519 |
| 1.2(2) | 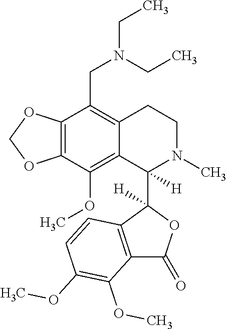 | 498.58 | 499 |

[symbol]

3

| | | | |
|---|---|---|---|
| 1.2(3) | 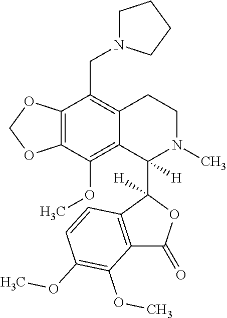 | 496.57 | 497 |
| 1.2(4) | 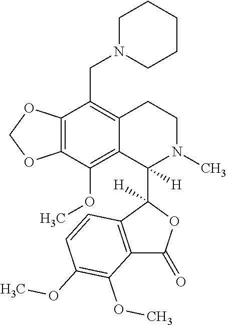 | 510.59 | 511 |
| 1.2(5) | 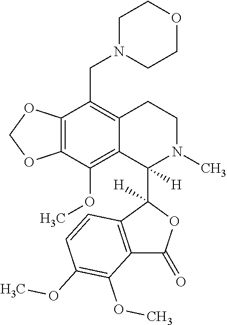 | 512.56 | 513 |
| 1.2(6) | 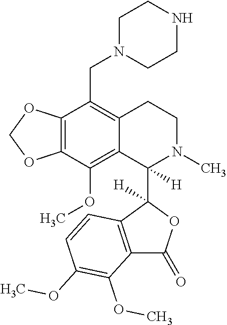 | 511.58 | 512 |
| 1.2(7) | 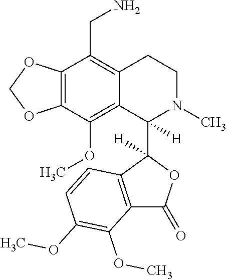 | 442.47 | 443 |

### Example 8

General method of obtaining 6,7-Dimethoxy-3-4-methoxy-6-methyl-9-(sulfamoyl)-5,6,7,8-tetrahydro-1,3 dioxolo 4,5-g isoquinoline-5-yl-3H-isobenzofuran-1-one 1.3. To that cooled to o s to chlorosulfonic acid (1 ml) during the mixing are added 103 mg (0.25 millimole) NSC. Mixture they intermix in the cold 0.5 h, after which transfer on the glacial solid is separated by centrifugation, they wash in icy water with the repeated centrifugation. Sulfochloride 1 (6) was obtained, dissolved in dioxane and are processed by 0.5 millimole of amine. The solution was intermixed 20 min, process by water, and the precipitated solid isolated by centrifugation, washed in water, dried, and recrystallized from isopropanol. Obtain 1.3, including 6,7-dimethoxy-3-4-methoxy-6-methyl-9-(morpholin-1-sulfonyl)-5,6,7,8-tetrahydro-1, 3 dioxolo 4,5-g isoquinoline-5-yl-3H-isobenzofuran-1-on 1.3 (5), NMR-1H (CDCl3, TMS): d 7.07 (Jo=8.4 Hz, IH, 5-H), d 6.40 (Jo=8.4 Hz, IH, 4-H), d 6.07 (J=I.5 Hz, 2′-H), d 6.06 (J=1.5 Hz, 2′-H), d 5.40 (J=4.8 Hz, IH, 3-H), d 4.37 (J=4.8 Hz, IH, 5′-H), s 4.10 (ZN, OCH3), s 4.09 (ZN, OCH3), s 3.88 (ZN, OCH3), m 3.72-3.77 (4H, CH2-O—CH2), m 3.15-3.25 (5H, CH2-O—CH2,7′-H), m 2.81-2.88 (IH, 7′-H), s 2.52 (ZN, 6′-CH3), m 2.33-2.41 (IH5 8′-H), m 2.11-2.20 (IH, 8′-H), NMR-13C (CDCl3, TMS): 167.87, 152.50, 148.55, 147.86, 143.77, 141.07, 133.93, 132.41, 119.78, 119.69, 118.50, 117.52, 111.18, 101.77, 81.34, 66.44, 62.30, 61.21, 59.67, 56.81, 49.35, 45.86, 45. 93, 24.96, etc of 6,7-dimethoxy-3- of 4-methoxy- of 6-methyl-9-(sulfamoyl)-5,6,7,8-tetrahydro-1,3 dioxolo 4,5-g isoquinoline-5-yl-3H-isobenzofuran-1-one 1.3, whose combinatorial library is represented in Table 4.

TABLE 4
combinatory library of 6,7-dimethoxy-3-4-methoxy-6-methyl-9-(sulfonyl)-
5,6,7,8-tetrahydro-1,3 dioxolo 4,5-g isoquinoline-5-yl-3H— of
isobenzofuran-1-one 1.3

| | | [symbol] | LC-MS, |
|---|---|---|---|
| [symbol] | [symbol] | (M) | m/z (M + H) |
| 1.3(1) | 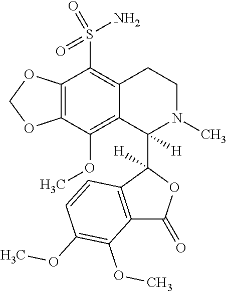 | 492.51 | 493 |
| 1.3(2) | 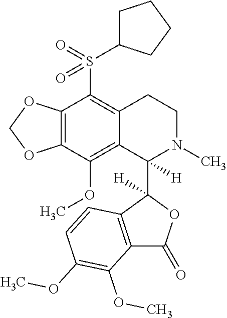 | 546.60 | 547 |
| 1.3(3) | 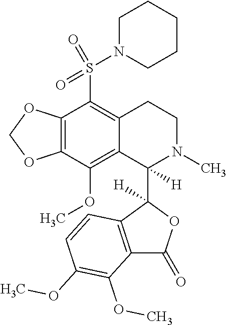 | 560.63 | 561 |
| 1.3(4) | 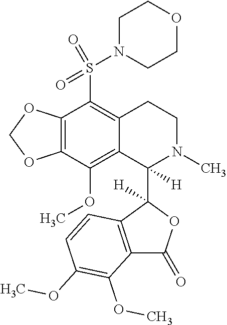 | 562.60 | 563 |

[symbol]

4

| | | | |
|---|---|---|---|
| 1.3(5) | 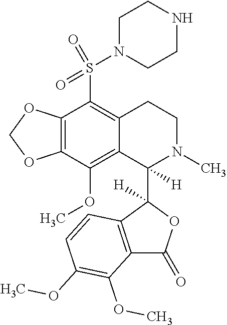 | 561.62 | 562 |
| 1.3(6) | 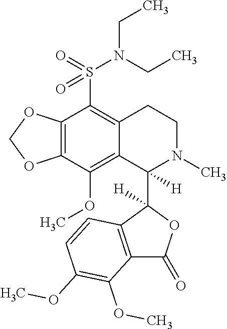 | 548.62 | 549 |
| 1.3(7) | 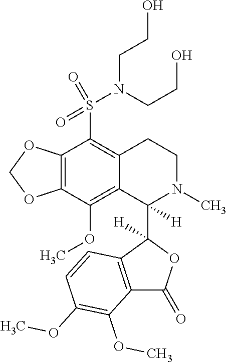 | 580.62 | 581 |

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. A method for inhibiting microbial replication, growth, and/or proliferation in the cells of a patient to be treated, comprising the steps of administering an effective amount of noscapine or a noscapine analog to inhibit microbial transport within the cells of the patient to be treated, wherein the noscapine analogue has one of the following formulas: 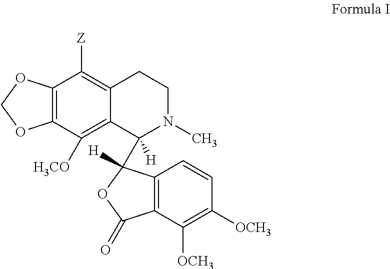
wherein Z is, individually, selected from the group consisting of alkyl, substituted alkyl, alkenyl, substituted alkenyl, heterocyclyl, substituted heterocyclyl, cycloalkyl, substituted cycloalkyl, aryl, substituted aryl, alkylaryl, substituted alkylaryl, arylalkyl, substituted arylalkyl, —OR′, —NR′R″, —CF₃, —CN, —C₂R′, —SR', —N₃, —C(═O)NR′R″, —NR′C(═O)R″, —C(═O)R′, —C(═O)OR′, —OC(═O)R′, —O(CR′R″)ᵣC(═O)R′, —O(CR′R″)ᵣNR″C(═O)R′, —O(CR′R″)ᵣNR″SO₂R′, —OC(═O)NR′R″, —NR′C(═O)OR″, —SO₂R′, —SO₂NR′R″, and —NR′SO₂R″,
where R′ and R″ are individually hydrogen, C₁-C₈ alkyl, cycloalkyl, heterocyclyl, aryl, or arylalkyl, and r is an integer from 1 to 6,
wherein the term “substituted” as applied to alkyl, aryl, cycloalkyl and the like refers to the substituents described above, starting with alkyl and ending with —NR′SO₂R″; 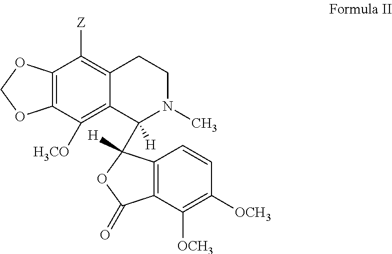
wherein Z is nitro, bromo, iodo, or fluoro, 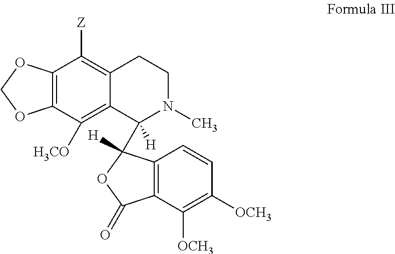
wherein Z is amino, and 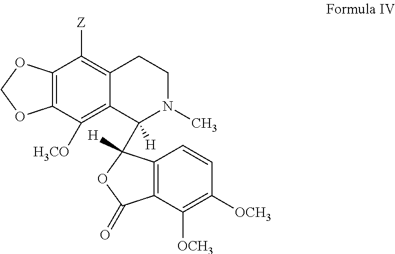
wherein Z is chloro,
and pharmaceutically-acceptable salts and prodrugs thereof.

2. The method of claim 1, wherein the compound is a compound of Formula I.

3. The method of claim 1, wherein the compound is a compound of Formula II.

4. The method of claim 1, wherein the compound is a compound of Formula III.

5. The method of claim 1, wherein the compound is a compound of Formula IV.

6. The method of claim 1, wherein the microbe is a virus.

7. The method of claim 6, wherein the virus is a retrovirus.

8. The method of claim 6, wherein the virus is a member of a a viral family selected from the group consisting of Adenoviridae, Papillomaviridae, Parvoviridae, Herpesviridae, Poxyiridae, Hepadnaviridae, Polyomaviridae, Influenzae, and Circoviridae.

9. The method of claim 6, wherein the virus is selected from the group consisting of HIV, ebola virus, polyoma virus, influenza virus, simian virus, herpes viruses, Human foamy virus (HFV), and Mason-Pfizer monkeyvirus (M-PMV).

10. The method of claim 6, further comprising the co-administration of an antiviral agent.

11. The method of claim 10, wherein the antiviral agent is selected from the group consisting of NRTIs, NNRTIs, VAP anti-idiotypic antibodies, CD4 and CCR5 receptor inhibitors, entry inhibitors, antisense oligonucleotides, ribozymes, protease inhibitors, neuraminidase inhibitors, tyrosine kinase inhibitors, PI-3 kinase inhibitors, and Interferons.

12. The method of any of claim 1, wherein the microbe is a bacteria.

13. The method of claim 12, wherein the bacteria is selected from the group consisting of Shingella species, Salmonella species, Actinobacillus species, Francisella tularensis spp., Campylobacter jejuni, Citrobacter freundii spp., Shigella flexneri, E. coli, Yersinia enterocolitica, Mycobacteria tuberculosis or related mycobacteria, Meningococcus, Chlamydia, Agrobacterium tumefaciens, Aquaspirillum, Bacillus, Bacteroides, Bordetella pertussis, Borrelia burgdorferi, Brucella, Burkholderia, Campylobacter, Chlamydia, Clostridium, Corynebacterium diptheriae, Coxiella burnetii, Deinococcus radiodurans, Enterococcus, Escherichia, Francisella tularemsis, Geobacillus, Haemophilus influenzae, Helicobacter pylori, Lactobacillus, Listeria monocytogenes, Mycobacterium, Mycoplasma, Neisseria meningitidis, Pseudomonas, Rickettsia, Salmonella, Shigella, Staphylococcus, Streptococcus, Streptomyces coelicolor, Vibro, and Yersinia.

14. The method of claim 12, further comprising the co-administration of an antibacterial agent.

15. The method of claim 10, wherein the antibacterial agent is selected from the group consisting of aminoglycosides, ansamycins, carbacephems, carbapenems, cephalosporins, glycopeptides, macrolides, monobactams, penicillins and beta-lactam antibiotics, quinolones, sulfonamides, tetracyclines, and antimicrobial peptides.

16. The method of claim 1, wherein the microbe is a fungi.

17. The method of claim 16, wherein the fungi is selected from the group consisting of Candida albicans, Paracoccidioides brasiliensis, Saccharomyces cerevisiae, and Schizosaccharomyces pombe.

18. The method of claim 16, further comprising the co-administration of an antifungal agent.

19. The method of claim 18, wherein the antifungal agent is selected from the group consisting of Amphotericin B, Itraconazole, Tebuconazole, Posaconazole, Ketoconazole, Fluconazole PO, Clotrimazole troche, Nystatin oral suspension, Voriconazole, Griseofulvin, Terbinafine, and Flucytosine.

20-38. (canceled)
